SEQUENCE LISTING (614 sequences)

SEQ ID NO: 1 - protein, 8 aa, synthetic construct
  REGION 1..8: note = Description of Sequence: Synthetic peptide
  SITE 1: note = Nle
  SITE 4: note = D-phenylalanine
  SITE 7: note = bAla
XDHXRWXK

SEQ ID NO: 2 - protein, 8 aa, synthetic construct
  REGION 1..8: note = Description of Sequence: Synthetic peptide
  SITE 1: note = Nle
  SITE 4: note = D-phenylalanine
  SITE 7: note = 1-amino-1-cyclohexanecarboxylic acid
XDHXRWXK

SEQ ID NO: 3 - protein, 8 aa, synthetic construct
  REGION 1..8: note = Description of Sequence: Synthetic peptide
  SITE 1: note = Nle
  SITE 4: note = D-phenylalanine
  SITE 7: note = 6-aminohexanoic acid
XCHXRWXC

SEQ ID NO: 4 - protein, 9 aa, synthetic construct
  REGION 1..9: note = Description of Sequence: Synthetic peptide
  SITE 1: note = D-phenylalanine
  SITE 4: note = D-phenylalanine
  SITE 8: note = D-cysteine
XCHXRWAXT

SEQ ID NO: 5 - protein, 9 aa, synthetic construct
  REGION 1..9: note = Description of Sequence: Synthetic peptide
  SITE 1: note = D-phenylalanine
  SITE 4: note = D-phenylalanine
  SITE 7: note = bAla
  SITE 8: note = D-cysteine
XCHXRWXXT

SEQ ID NO: 6 - protein, 9 aa, synthetic construct
  REGION 1..9: note = Description of Sequence: Synthetic peptide
  SITE 1: note = D-phenylalanine
  SITE 4: note = D-phenylalanine
  SITE 7: note = 4Abu
  SITE 8: note = D-cysteine
XCHXRWXXT

SEQ ID NO: 7 - protein, 8 aa, synthetic construct
  REGION 1..8: note = Description of Sequence: Synthetic peptide
  SITE 1: note = Nle
  SITE 4: note = D-phenylalanine
  SITE 7: note = 5-aminopentanoic acid
XCHXRWXC

SEQ ID NO: 8 - protein, 8 aa, synthetic construct
  REGION 1..8: note = Description of Sequence: Synthetic peptide
  SITE 1: note = Nle
  SITE 4: note = D-phenylalanine
  SITE 7: note = 5-aminopentanoic acid
XDHXRWXK

SEQ ID NO: 9 - protein, 8 aa, synthetic construct
  REGION 1..8: note = Description of Sequence: Synthetic peptide
  SITE 1: note = 1-amino-1-cyclohexanecarboxylic acid
  SITE 4: note = D-phenylalanine
  SITE 7: note = 4Abu
XDHXRWXK

SEQ ID NO: 10 - protein, 8 aa, synthetic construct
  REGION 1..8: note = Description of Sequence: Synthetic peptide
  SITE 1: note = D-b-(2-naphthyl) alanine
  SITE 4: note = D-phenylalanine
  SITE 7: note = 4Abu
XDHXRWXK

SEQ ID NO: 11 - protein, 8 aa, synthetic construct
  REGION 1..8: note = Description of Sequence: Synthetic peptide
  SITE 1: note = b-cyclohexylalanine
  SITE 4: note = D-phenylalanine
  SITE 7: note = 4Abu
XDHXRWXK

SEQ ID NO: 12 - protein, 8 aa, synthetic construct
  REGION 1..8: note = Description of Sequence: Synthetic peptide
  SITE 1: note = Nle
  SITE 4: note = D-phenylalanine
  SITE 7: note = 4Abu
XDHXRWXK

SEQ ID NO: 13 - protein, 9 aa, synthetic construct
  REGION 1..9: note = Description of Sequence: Synthetic peptide
  SITE 6: note = D-phenylalanine
RGCEHXRWC

SEQ ID NO: 14 - protein, 8 aa, synthetic construct
  REGION 1..8: note = Description of Sequence: Synthetic peptide
  SITE 1: note = Nle
  SITE 3: note = bAla
  SITE 5: note = D-phenylalanine
XCXHXRWC

SEQ ID NO: 15 - protein, 8 aa, synthetic construct
  REGION 1..8: note = Description of Sequence: Synthetic peptide
  SITE 1: note = Nle
  SITE 3: note = 4Abu
  SITE 5: note = D-phenylalanine
XCXHXRWC

SEQ ID NO: 16 - protein, 8 aa, synthetic construct
  REGION 1..8: note = Description of Sequence: Synthetic peptide
  SITE 1: note = Nle
  SITE 3: note = Aib
  SITE 5: note = D-phenylalanine
XCXHXRWC

SEQ ID NO: 17 - protein, 8 aa, synthetic construct
  REGION 1..8: note = Description of Sequence: Synthetic peptide
  SITE 1: note = Nle
  SITE 5: note = D-phenylalanine
XCGHXRWC

SEQ ID NO: 18 - protein, 8 aa, synthetic construct
  REGION 1..8: note = Description of Sequence: Synthetic peptide
  SITE 1: note = Nle
  SITE 2: note = D-cysteine
  SITE 5: note = D-phenylalanine
XXAHXRWC

SEQ ID NO: 19 - protein, 8 aa, synthetic construct
  REGION 1..8: note = Description of Sequence: Synthetic peptide
  SITE 1: note = Nle
  SITE 2: note = D-cysteine
  SITE 3: note = D-Alanine
  SITE 5: note = D-phenylalanine
XXXHXRWC

SEQ ID NO: 20 - protein, 8 aa, synthetic construct
  REGION 1..8: note = Description of Sequence: Synthetic peptide
  SITE 1: note = Nle
  SITE 2: note = D-cysteine
  SITE 3: note = bAla
  SITE 5: note = D-phenylalanine
XXXHXRWC

SEQ ID NO: 21 - protein, 8 aa, synthetic construct
  REGION 1..8: note = Description of Sequence: Synthetic peptide
  SITE 1: note = Nle
  SITE 2: note = D-cysteine
  SITE 3: note = 4Abu
  SITE 5: note = D-phenylalanine
XXXHXRWC

SEQ ID NO: 22 - protein, 8 aa, synthetic construct
  REGION 1..8: note = Description of Sequence: Synthetic peptide
  SITE 1: note = Nle
  SITE 2: note = D-cysteine
  SITE 3: note = Aib
  SITE 5: note = D-phenylalanine
XXXHXRWC

SEQ ID NO: 23 - protein, 8 aa, synthetic construct
  REGION 1..8: note = Description of Sequence: Synthetic peptide
  SITE 1: note = Nle
  SITE 2: note = D-cysteine
  SITE 5: note = D-phenylalanine
XXGHXRWC

SEQ ID NO: 24 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 8: note = D-cysteine
XCXHXRWX

SEQ ID NO: 25 - protein, 8 aa, synthetic construct
  REGION 1..8: note = Description of Sequence: Synthetic peptide
  SITE 1: note = Nle
  SITE 3: note = bAla
  SITE 5: note = D-phenylalanine
  SITE 8: note = D-cysteine
XCXHXRWX

SEQ ID NO: 26 - protein, 8 aa, synthetic construct
  REGION 1..8: note = Description of Sequence: Synthetic peptide
  SITE 1: note = Nle
  SITE 3: note = 4Abu
  SITE 5: note = D-phenylalanine
  SITE 8: note = D-cysteine
XCXHXRWX

SEQ ID NO: 27 - protein, 8 aa, synthetic construct
  REGION 1..8: note = Description of Sequence: Synthetic peptide
  SITE 1: note = Nle
  SITE 3: note = Aib
  SITE 5: note = D-phenylalanine
  SITE 8: note = D-cysteine
XCXHXRWX

SEQ ID NO: 28 - protein, 8 aa, synthetic construct
  REGION 1..8: note = Description of Sequence: Synthetic peptide
  SITE 1: note = Nle
  SITE 5: note = D-phenylalanine
  SITE 8: note = D-cysteine
XCGHXRWX

SEQ ID NO: 29 - protein, 8 aa, synthetic construct
  REGION 1..8: note = Description of Sequence: Synthetic peptide
  SITE 1: note = Nle
  SITE 2: note = D-cysteine
  SITE 5: note = D-phenylalanine
  SITE 8: note = D-cysteine
XXAHXRWX

SEQ ID NO: 30 - protein, 8 aa, synthetic construct
  REGION 1..8: note = Description of Sequence: Synthetic peptide
  SITE 1: note = Nle
  SITE 2: note = D-cysteine
  SITE 3: note = D-Alanine
  SITE 5: note = D-phenylalanine
  SITE 8: note = D-cysteine
XCAHFRWC

SEQ ID NO: 31 - protein, 8 aa, synthetic construct
  REGION 1..8: note = Description of Sequence: Synthetic peptide
  SITE 1: note = Nle
  SITE 2: note = D-cysteine
  SITE 3: note = bAla
  SITE 5: note = D-phenylalanine
  SITE 8: note = D-cysteine
XCXHFRWC

SEQ ID NO: 32 - protein, 8 aa, synthetic construct
  REGION 1..8: note = Description of Sequence: Synthetic peptide
  SITE 1: note = Nle
  SITE 2: note = D-cysteine
  SITE 3: note = 4Abu
  SITE 5: note = D-phenylalanine
  SITE 8: note = D-cysteine
XCXHFRWC

SEQ ID NO: 33 - protein, 8 aa, synthetic construct
  REGION 1..8: note = Description of Sequence: Synthetic peptide
  SITE 1: note = Nle
  SITE 2: note = D-cysteine
  SITE 3: note = Aib
  SITE 5: note = D-phenylalanine
  SITE 8: note = D-cysteine
XCXHFRWC

SEQ ID NO: 34 - protein, 8 aa, synthetic construct
  REGION 1..8: note = Description of Sequence: Synthetic peptide
  MOD_RES 1: note = Octahydroindole-2-carboxylic acid
  SITE 4: note = D-phenylalanine
  SITE 7: note = 4Abu
XDHFRWXK

SEQ ID NO: 35 - protein, 8 aa, synthetic construct
  REGION 1..8: note = Description of Sequence: Synthetic peptide
  SITE 1: note = Cyclohexylglycine
  SITE 4: note = D-phenylalanine
  SITE 7: note = 4Abu
XDHFRWXK

SEQ ID NO: 36 - protein, 8 aa, synthetic construct
  REGION 1..8: note = Description of Sequence: Synthetic peptide
  SITE 1: note = homo-cyclohexylalanine
  SITE 4: note = D-phenylalanine
  SITE 7: note = 4Abu
XDHFRWXK

SEQ ID NO: 37 - protein, 8 aa, synthetic construct
  REGION 1..8: note = Description of Sequence: Synthetic peptide
  SITE 1: note = D-b-cyclohexylalanine
  SITE 4: note = D-phenylalanine
  SITE 7: note = 4Abu
XDHFRWXK

SEQ ID NO: 38 - protein, 8 aa, synthetic construct
  REGION 1..8: note = Description of Sequence: Synthetic peptide
  SITE 1: note = D-homo-cyclohexylalanine
  SITE 4: note = D-phenylalanine
  SITE 7: note = 4Abu
XDHXRWXK

SEQ ID NO: 39 - protein, 8 aa, synthetic construct
  REGION 1..8: note = Description of Sequence: Synthetic peptide
  SITE 1: note = Nipecotic acid
  SITE 4: note = D-phenylalanine
  SITE 7: note = 4Abu
XDHFRWXK

SEQ ID NO: 40 - protein, 8 aa, synthetic construct
  REGION 1..8: note = Description of Sequence: Synthetic peptide
  SITE 4: note = D-phenylalanine
  SITE 7: note = 4Abu
PDHFRWXK

SEQ ID NO: 41 - protein, 8 aa, synthetic construct
  REGION 1..8: note = Description of Sequence: Synthetic peptide
  SITE 4: note = D-phenylalanine
  SITE 7: note = 4Abu
LDHFRWXK

SEQ ID NO: 42 - protein, 8 aa, synthetic construct
  REGION 1..8: note = Description of Sequence: Synthetic peptide
  SITE 4: note = D-phenylalanine
  SITE 7: note = 4Abu
FDHFRWXK

SEQ ID NO: 43 - protein, 8 aa, synthetic construct
  REGION 1..8: note = Description of Sequence: Synthetic peptide
  SITE 1: note = D-phenylalanine
  SITE 4: note = D-phenylalanine
  SITE 7: note = 4Abu
FDHFRWXK

SEQ ID NO: 44 - protein, 8 aa, synthetic construct
  REGION 1..8: note = Description of Sequence: Synthetic peptide
  SITE 1: note = D-Cyclohexylglycine
  SITE 4: note = D-phenylalanine
  SITE 7: note = 4Abu
XDHFRWXK

SEQ ID NO: 45 - protein, 8 aa, synthetic construct
  REGION 1..8: note = Description of Sequence: Synthetic peptide
  SITE 1: note = b-cyclohexylalanine
  SITE 4: note = D-phenylalanine
  SITE 7: note = 4Abu
XDHFRWXK

SEQ ID NO: 46 - protein, 8 aa, synthetic construct
  REGION 1..8: note = Description of Sequence: Synthetic peptide
  SITE 1: note = b-cyclohexylalanine
  SITE 4: note = D-phenylalanine
  SITE 7: note = 4Abu
XDHXRWXK

SEQ ID NO: 47 - protein, 8 aa, synthetic construct
  REGION 1..8: note = Description of Sequence: Synthetic peptide
  SITE 4: note = D-phenylalanine
  SITE 7: note = 4Abu
FDHFRWXK

SEQ ID NO: 48 - protein, 8 aa, synthetic construct
  REGION 1..8: note = Description of Sequence: Synthetic peptide
  SITE 1: note = Beta-hMet
  SITE 4: note = D-phenylalanine
  SITE 7: note = 4Abu
XDHFRWXK

SEQ ID NO: 49 - protein, 8 aa, synthetic construct
  REGION 1..8: note = Description of Sequence: Synthetic peptide
  SITE 1: note = 4Abu
  SITE 4: note = D-phenylalanine
  SITE 7: note = 4Abu
XDHFRWXK

SEQ ID NO: 50 - protein, 8 aa, synthetic construct
  REGION 1..8: note = Description of Sequence: Synthetic peptide
  SITE 1: note = b-cyclohexylalanine
  SITE 4: note = D-phenylalanine
  SITE 6: note = D-Tryptophan
XDHFRXAK

SEQ ID NO: 51 - protein, 8 aa, synthetic construct
  REGION 1..8: note = Description of Sequence: Synthetic peptide
  SITE 1: note = homo-cyclohexylalanine
  SITE 4: note = D-phenylalanine
  SITE 6: note = D-Tryptophan
XDHFRXAK

SEQ ID NO: 52 - protein, 8 aa, synthetic construct
  REGION 1..8: note = Description of Sequence: Synthetic peptide
  SITE 4: note = D-phenylalanine
  SITE 6: note = D-Tryptophan
LDHFRWAK

SEQ ID NO: 53 - protein, 8 aa, synthetic construct
  REGION 1..8: note = Description of Sequence: Synthetic peptide
  SITE 4: note = D-phenylalanine
  SITE 6: note = D-Tryptophan
LDHFRWAK

SEQ ID NO: 54 - protein, 8 aa, synthetic construct
  REGION 1..8: note = Description of Sequence: Synthetic peptide
  SITE 4: note = D-phenylalanine
  SITE 6: note = D-Tryptophan
FDHXRXAK

SEQ ID NO: 55 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = D-Alanine
XDHFRWXK

SEQ ID NO: 56 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = bAla
XDHFRWXK

SEQ ID NO: 57 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 4Abu
XDHFRWXK

SEQ ID NO: 58 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 7-aminoheptanoic acid
XDHXRWXK

SEQ ID NO: 59 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 5-aminopentanoic acid
XDHXRWXK

SEQ ID NO: 60 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 5-aminopentanoic acid
XCHXRXXC

SEQ ID NO: 61 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 4Abu
XCHXRWXC

SEQ ID NO: 62 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 6-aminohexanoic acid
XCHXRWXC

SEQ ID NO: 63 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = bAla
XCHFRWXC

SEQ ID NO: 64 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = D-Alanine
XCHFRWXC

SEQ ID NO: 65 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
XCAHXRWC

SEQ ID NO: 66 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
  SITE 7: note = b-(2-naphthyl) alanine
XCAHXRXC

SEQ ID NO: 67 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
  SITE 7: note = b-(1-naphthyl) alanine
XCAHXRXC

SEQ ID NO: 68 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 7: note = b-(2-naphthyl) alanine
XCAHFRXC

SEQ ID NO: 69 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
XCAHFRWC

SEQ ID NO: 70 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 7: note = b-(2-naphthyl) alanine
XCAHFRXC

SEQ ID NO: 71 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 7: note = b-(1-naphthyl) alanine
XCAHFRXC

SEQ ID NO: 72 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 7: note = 3-benzothienylalanine
XCAHFRXC

SEQ ID NO: 73 - protein, 8 aa, synthetic construct
  REGION 1..8: note = Description of Sequence: Synthetic peptide
  SITE 1: note = Nle
  SITE 3: note = D-Glutamic acid
  SITE 5: note = D-phenylalanine
XCEHFRWC

SEQ ID NO: 74 - protein, 8 aa, synthetic construct
  REGION 1..8: note = Description of Sequence: Synthetic peptide
  SITE 1: note = Nle
  SITE 4: note = D-phenylalanine
  SITE 7: note = D-Alanine
XDHFRWAK

SEQ ID NO: 75 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
  SITE 7: note = 3-benzothienylalanine
XCAHXRXC

SEQ ID NO: 76 - protein, 8 aa, synthetic construct
  REGION 1..8: note = Description of Sequence: Synthetic peptide
  SITE 1: note = Nle
  SITE 2: note = Penicillamine
  SITE 3: note = D-Alanine
  SITE 5: note = D-phenylalanine
XXAHFRWC

SEQ ID NO: 77 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 8: note = Penicillamine
XCAHFRWX

SEQ ID NO: 78 - protein, 8 aa, synthetic construct
  REGION 1..8: note = Description of Sequence: Synthetic peptide
  SITE 1: note = Nle
  SITE 2: note = Penicillamine
  SITE 3: note = D-Alanine
  SITE 5: note = D-phenylalanine
  SITE 8: note = Penicillamine
XXAHFRWX

SEQ ID NO: 79 - protein, 9 aa, synthetic construct
  REGION 1..9: note = Description of Sequence: Synthetic peptide
  SITE 1: note = D-phenylalanine
  SITE 4: note = D-phenylalanine
  SITE 7: note = bAla
  SITE 8: note = D-cysteine
FCHFRWXCT

SEQ ID NO: 80 - protein, 9 aa, synthetic construct
  REGION 1..9: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
XCHXRWXXT

SEQ ID NO: 81 - protein, 9 aa, synthetic construct
  REGION 1..9: note = Description of Sequence: Synthetic peptide
  SITE 1: note = D-phenylalanine
  SITE 4: note = D-phenylalanine
  SITE 6: note = 4,4 -biphenylalanine
  SITE 7: note = bAla
  SITE 8: note = D-cysteine
FCHFRXXXT

SEQ ID NO: 82 - protein, 9 aa, synthetic construct
  REGION 1..9: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
XCHXRWXXT

SEQ ID NO: 83 - protein, 9 aa, synthetic construct
  REGION 1..9: note = Description of Sequence: Synthetic peptide
  SITE 1: note = D-phenylalanine
  SITE 4: note = D-phenylalanine
  SITE 6: note = 4,4 -biphenylalanine
  SITE 7: note = bAla
  SITE 8: note = D-cysteine
XCHXRXXXT

SEQ ID NO: 84 - protein, 9 aa, synthetic construct
  REGION 1..9: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
XCHXRXXXT

SEQ ID NO: 85 - protein, 8 aa, synthetic construct
  REGION 1..8: note = Description of Sequence: Synthetic peptide
  SITE 1: note = Nle
  SITE 4: note = D-phenylalanine
  SITE 7: note = 5-aminopentanoic acid
XCHXRWXC

SEQ ID NO: 86 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
XDXHXRWK

SEQ ID NO: 87 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 7: note = 3-benzothienylalanine
XDXHXRXK

SEQ ID NO: 88 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
  SITE 8: note = Penicillamine
XCXHXRWX

SEQ ID NO: 89 - protein, 8 aa, synthetic construct
  REGION 1..8: note = Description of Sequence: Synthetic peptide
  SITE 1: note = Nle
  SITE 3: note = D-Abu
  SITE 5: note = D-phenylalanine
XCXHXRWC

SEQ ID NO: 90 - protein, 8 aa, synthetic construct
  REGION 1..8: note = Description of Sequence: Synthetic peptide
  SITE 1: note = Nle
  SITE 3: note = D-Valine
  SITE 5: note = D-phenylalanine
XCXHXRWC

SEQ ID NO: 91 - protein, 8 aa, synthetic construct
  REGION 1..8: note = Description of Sequence: Synthetic peptide
  SITE 1: note = Nle
  SITE 3: note = D-Isoleucine
  SITE 5: note = D-phenylalanine
XCXHXRWC

SEQ ID NO: 92 - protein, 8 aa, synthetic construct
  REGION 1..8: note = Description of Sequence: Synthetic peptide
  SITE 1: note = Nle
  SITE 3: note = D-leucine
  SITE 5: note = D-phenylalanine
XCLHFRWC

SEQ ID NO: 93 - protein, 8 aa, synthetic construct
  REGION 1..8: note = Description of Sequence: Synthetic peptide
  SITE 1: note = Nle
  SITE 3: note = D-tert-Leucine
  SITE 5: note = D-phenylalanine
XCLHFRWC

SEQ ID NO: 94 - protein, 8 aa, synthetic construct
  REGION 1..8: note = Description of Sequence: Synthetic peptide
  SITE 1: note = Nle
  SITE 3: note = D-b-cyclohexylalanine
  SITE 5: note = D-phenylalanine
XCXHFRWC

SEQ ID NO: 95 - protein, 8 aa, synthetic construct
  REGION 1..8: note = Description of Sequence: Synthetic peptide
  SITE 1: note = Nle
  SITE 2: note = Penicillamine
  SITE 4: note = D-phenylalanine
  SITE 7: note = 4Abu
XXHFRWXC

SEQ ID NO: 96 - protein, 8 aa, synthetic construct
  REGION 1..8: note = Description of Sequence: Synthetic peptide
  SITE 1: note = Nle
  SITE 4: note = D-phenylalanine
  SITE 7: note = 4Abu
  SITE 8: note = Penicillamine
XCHFRWXX

SEQ ID NO: 97 - protein, 8 aa, synthetic construct
  REGION 1..8: note = Description of Sequence: Synthetic peptide
  SITE 1: note = Nle
  SITE 2: note = Penicillamine
  SITE 4: note = D-phenylalanine
  SITE 7: note = 4Abu
  SITE 8: note = Penicillamine
XXHFRWXX

SEQ ID NO: 98 - protein, 8 aa, synthetic construct
  REGION 1..8: note = Description of Sequence: Synthetic peptide
  SITE 4: note = D-phenylalanine
  SITE 7: note = 4Abu
LCHFRWXC

SEQ ID NO: 99 - protein, 8 aa, synthetic construct
  REGION 1..8: note = Description of Sequence: Synthetic peptide
  SITE 1: note = b-cyclohexylalanine
  SITE 4: note = D-phenylalanine
  SITE 7: note = 4Abu
XCHFRWXC

SEQ ID NO: 100 - protein, 8 aa, synthetic construct
  REGION 1..8: note = Description of Sequence: Synthetic peptide
  SITE 4: note = D-phenylalanine
  SITE 7: note = 4Abu
ICHFRWXC

SEQ ID NO: 101 - protein, 8 aa, synthetic construct
  REGION 1..8: note = Description of Sequence: Synthetic peptide
  SITE 4: note = D-phenylalanine
  SITE 7: note = 4Abu
FCHFRWXC

SEQ ID NO: 102 - protein, 8 aa, synthetic construct
  REGION 1..8: note = Description of Sequence: Synthetic peptide
  SITE 4: note = D-phenylalanine
  SITE 7: note = 4Abu
VCHXRWXC

SEQ ID NO: 103 - protein, 8 aa, synthetic construct
  REGION 1..8: note = Description of Sequence: Synthetic peptide
  SITE 1: note = b-(2-naphthyl) alanine
  SITE 4: note = D-phenylalanine
  SITE 7: note = 4Abu
XCHXRWXC

SEQ ID NO: 104 - protein, 8 aa, synthetic construct
  REGION 1..8: note = Description of Sequence: Synthetic peptide
  SITE 1: note = Nle
  SITE 4: note = D-phenylalanine
  SITE 7: note = 4Abu
XCHXRWXC

SEQ ID NO: 105 - protein, 8 aa, synthetic construct
  REGION 1..8: note = Description of Sequence: Synthetic peptide
  SITE 4: note = D-phenylalanine
  SITE 7: note = 4Abu
FCHXRWXC

SEQ ID NO: 106 - protein, 8 aa, synthetic construct
  REGION 1..8: note = Description of Sequence: Synthetic peptide
  SITE 1: note = Nle
  SITE 3: note = 3-Pal
  SITE 4: note = D-phenylalanine
  SITE 7: note = 4Abu
XCXXRWXC

SEQ ID NO: 107 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
XCXHXRWC

SEQ ID NO: 108 - protein, 8 aa, synthetic construct
  REGION 1..8: note = Description of Sequence: Synthetic peptide
  SITE 1: note = Nle
  SITE 6: note = D-Tryptophan
  SITE 7: note = 4Abu
XCHFRXXC

SEQ ID NO: 109 - protein, 8 aa, synthetic construct
  REGION 1..8: note = Description of Sequence: Synthetic peptide
  SITE 1: note = Nle
  SITE 4: note = D-b-(2-naphthyl) alanine
XDHXRWAK

SEQ ID NO: 110 - protein, 8 aa, synthetic construct
  REGION 1..8: note = Description of Sequence: Synthetic peptide
  SITE 1: note = Nle
  SITE 4: note = D-b-(2-naphthyl) alanine
  SITE 7: note = bAla
XDHXRWXK

SEQ ID NO: 111 - protein, 8 aa, synthetic construct
  REGION 1..8: note = Description of Sequence: Synthetic peptide
  SITE 1: note = Nle
  SITE 4: note = D-b-(2-naphthyl) alanine
  SITE 7: note = 4Abu
XCHXRWXC

SEQ ID NO: 112 - protein, 8 aa, synthetic construct
  REGION 1..8: note = Description of Sequence: Synthetic peptide
  SITE 1: note = Nle
  SITE 4: note = D-b-(2-naphthyl) alanine
  SITE 7: note = 6-aminohexanoic acid
XCHXRWXC

SEQ ID NO: 113 - protein, 8 aa, synthetic construct
  REGION 1..8: note = Description of Sequence: Synthetic peptide
  SITE 4: note = D-b-(2-naphthyl) alanine
  SITE 7: note = 4Abu
FDHXRWXK

SEQ ID NO: 114 - protein, 8 aa, synthetic construct
  REGION 1..8: note = Description of Sequence: Synthetic peptide
  SITE 1: note = b-cyclohexylalanine
  SITE 4: note = D-b-(2-naphthyl) alanine
  SITE 7: note = 4Abu
XDHXRWXK

SEQ ID NO: 115 - protein, 8 aa, synthetic construct
  REGION 1..8: note = Description of Sequence: Synthetic peptide
  SITE 1: note = Nle
  SITE 4: note = D-phenylalanine
  SITE 7: note = bAla
XDHXRWXK

SEQ ID NO: 116 - protein, 8 aa, synthetic construct
  REGION 1..8: note = Description of Sequence: Synthetic peptide
  SITE 1: note = Nle
  SITE 4: note = D-phenylalanine
  SITE 7: note = 6-aminohexanoic acid
XCHXRWXC

SEQ ID NO: 117 - protein, 9 aa, synthetic construct
  REGION 1..9: note = Description of Sequence: Synthetic peptide
  SITE 1: note = D-phenylalanine
  SITE 4: note = D-phenylalanine
  SITE 8: note = D-cysteine
XCHXRWAXT

SEQ ID NO: 118 - protein, 9 aa, synthetic construct
  REGION 1..9: note = Description of Sequence: Synthetic peptide
  SITE 1: note = D-phenylalanine
  SITE 4: note = D-phenylalanine
  SITE 7: note = bAla
  SITE 8: note = D-cysteine
XCHXRWXXT

SEQ ID NO: 119 - protein, 9 aa, synthetic construct
  REGION 1..9: note = Description of Sequence: Synthetic peptide
  SITE 1: note = D-phenylalanine
  SITE 4: note = D-phenylalanine
  SITE 7: note = 4Abu
  SITE 8: note = D-cysteine
XCHXRWXXT

SEQ ID NO: 120 - protein, 8 aa, synthetic construct
  REGION 1..8: note = Description of Sequence: Synthetic peptide
  SITE 1: note = Nle
  SITE 4: note = D-phenylalanine
  SITE 7: note = 5-aminopentanoic acid
XCHXRWXC

SEQ ID NO: 121 - protein, 8 aa, synthetic construct
  REGION 1..8: note = Description of Sequence: Synthetic peptide
  SITE 1: note = Nle
  SITE 4: note = D-phenylalanine
  SITE 7: note = 5-aminopentanoic acid
XDHXRWXK

SEQ ID NO: 122 - protein, 8 aa, synthetic construct
  REGION 1..8: note = Description of Sequence: Synthetic peptide
  SITE 1: note = b-cyclohexylalanine
  SITE 4: note = D-phenylalanine
  SITE 7: note = 4Abu
XDHXRWXK

SEQ ID NO: 123 - protein, 8 aa, synthetic construct
  REGION 1..8: note = Description of Sequence: Synthetic peptide
  SITE 1: note = Nle
  SITE 4: note = D-phenylalanine
  SITE 7: note = 4Abu
XDHXRWXK

SEQ ID NO: 124 - protein, 8 aa, synthetic construct
  REGION 1..8: note = Description of Sequence: Synthetic peptide
  SITE 1: note = Cyclohexylglycine
  SITE 4: note = D-phenylalanine
  SITE 7: note = 4Abu
XDHXRWXK

SEQ ID NO: 125 - protein, 8 aa, synthetic construct
  REGION 1..8: note = Description of Sequence: Synthetic peptide
  SITE 1: note = D-b-cyclohexylalanine
  SITE 4: note = D-phenylalanine
  SITE 7: note = 4Abu
XDHXRWXK

SEQ ID NO: 126 - protein, 8 aa, synthetic construct
  REGION 1..8: note = Description of Sequence: Synthetic peptide
  SITE 1: note = homo-cyclohexylalanine
  SITE 4: note = D-phenylalanine
  SITE 7: note = 4Abu
XDHXRWXK

SEQ ID NO: 127 - protein, 8 aa, synthetic construct
  REGION 1..8: note = Description of Sequence: Synthetic peptide
  SITE 1: note = D-Cyclohexylglycine
  SITE 4: note = D-phenylalanine
  SITE 7: note = 4Abu
XDHXRWXK

SEQ ID NO: 128 - protein, 8 aa, synthetic construct
  REGION 1..8: note = Description of Sequence: Synthetic peptide
  SITE 4: note = D-phenylalanine
  SITE 7: note = 4Abu
FDHXRWXK

SEQ ID NO: 129 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 4Abu
XCHXRXXC

SEQ ID NO: 130 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = 6-aminohexanoic acid
XCHXRXXC

SEQ ID NO: 131 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = bAla
XCHXRXXC

SEQ ID NO: 132 - protein, 8 aa, synthetic construct
  REGION 1..8: note = Description of Sequence: Synthetic peptide
  SITE 1: note = Nle
  SITE 4: note = D-phenylalanine
  SITE 6: note = D-Tryptophan
  SITE 7: note = D-Alanine
XCHXRXXC

SEQ ID NO: 133 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
XCXHXRWC

SEQ ID NO: 134 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
  SITE 7: note = b-(2-naphthyl) alanine
XCXHXRXC

SEQ ID NO: 135 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
  SITE 7: note = b-(1-naphthyl) alanine
XCXHXRXC

SEQ ID NO: 136 - protein, 8 aa, synthetic construct
  REGION 1..8: note = Description of Sequence: Synthetic peptide
  SITE 1: note = Nle
  SITE 3: note = D-Alanine
  SITE 5: note = D-b-(2-naphthyl) alanine
  SITE 7: note = 3-benzothienylalanine
XCAHXRXC

SEQ ID NO: 137 - protein, 8 aa, synthetic construct
  REGION 1..8: note = Description of Sequence: Synthetic peptide
  SITE 1: note = Nle
  SITE 2: note = Penicillamine
  SITE 3: note = D-Alanine
  SITE 5: note = D-phenylalanine
XXAHXRWC

SEQ ID NO: 138 - protein, 8 aa, synthetic construct
  REGION 1..8: note = Description of Sequence: Synthetic peptide
  SITE 1: note = Nle
  SITE 4: note = D-phenylalanine
  SITE 7: note = 4Abu
  SITE 8: note = Penicillamine
XCHFRWXX

SEQ ID NO: 139 - protein, 8 aa, synthetic construct
  REGION 1..8: note = Description of Sequence: Synthetic peptide
  SITE 3: note = D-Alanine
  SITE 5: note = D-b-(2-naphthyl) alanine
RCAHXRWC

SEQ ID NO: 140 - protein, 8 aa, synthetic construct
  REGION 1..8: note = Description of Sequence: Synthetic peptide
  SITE 3: note = D-Alanine
  SITE 5: note = D-phenylalanine
RCAHFRWC

SEQ ID NO: 141 - protein, 8 aa, synthetic construct
  REGION 1..8: note = Description of Sequence: Synthetic peptide
  SITE 1: note = D-arginine
  SITE 3: note = D-Alanine
  SITE 5: note = D-phenylalanine
RCAHFRWC

SEQ ID NO: 142 - protein, 8 aa, synthetic construct
  REGION 1..8: note = Description of Sequence: Synthetic peptide
  SITE 1: note = D-arginine
  SITE 3: note = D-Alanine
  SITE 5: note = D-phenylalanine
  SITE 8: note = Penicillamine
RCAHRRWX

SEQ ID NO: 143 - protein, 8 aa, synthetic construct
  REGION 1..8: note = Description of Sequence: Synthetic peptide
  SITE 1: note = D-arginine
  SITE 4: note = D-phenylalanine
  SITE 7: note = 4Abu
  SITE 8: note = Penicillamine
RCHFRWXX

SEQ ID NO: 144 - protein, 8 aa, synthetic construct
  REGION 1..8: note = Description of Sequence: Synthetic peptide
  SITE 4: note = D-phenylalanine
  SITE 7: note = 4Abu
  SITE 8: note = Penicillamine
RCHFRWXX

SEQ ID NO: 145 - protein, 8 aa, synthetic construct
  REGION 1..8: note = Description of Sequence: Synthetic peptide
  SITE 3: note = D-Alanine
  SITE 5: note = D-phenylalanine
  SITE 8: note = Penicillamine
RCAHFRWX

SEQ ID NO: 146 - protein, 8 aa, synthetic construct
  REGION 1..8: note = Description of Sequence: Synthetic peptide
  SITE 1: note = D-arginine
  SITE 4: note = D-phenylalanine
RDHFRWAK

SEQ ID NO: 147 - protein, 8 aa, synthetic construct
  REGION 1..8: note = Description of Sequence: Synthetic peptide
  SITE 4: note = D-phenylalanine
RDHFRWAK

SEQ ID NO: 148 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
ACXHFRWGC

SEQ ID NO: 149 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
  SITE 8: note = D-Alanine
XCXHXRWXC

SEQ ID NO: 150 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
  SITE 8: note = bAla
XCXHXRWXC

SEQ ID NO: 151 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
  SITE 8: note = 4Abu
XCXHXRWXC

SEQ ID NO: 152 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
  SITE 8: note = 5-aminopentanoic acid
XCXHXRWXC

SEQ ID NO: 153 - protein, 8 aa, synthetic construct
  REGION 1..8: note = Description of Sequence: Synthetic peptide
  SITE 4: note = D-phenylalanine
CEHXRWAC

SEQ ID NO: 154 - protein, 8 aa, synthetic construct
  REGION 1..8: note = Description of Sequence: Synthetic peptide
  SITE 4: note = D-phenylalanine
  SITE 6: note = b-(2-naphthyl) alanine
CEHXRXAC

SEQ ID NO: 155 - protein, 8 aa, synthetic construct
  REGION 1..8: note = Description of Sequence: Synthetic peptide
  SITE 2: note = D-Alanine
  SITE 4: note = D-phenylalanine
CXHXRWAC

SEQ ID NO: 156 - protein, 8 aa, synthetic construct
  REGION 1..8: note = Description of Sequence: Synthetic peptide
  SITE 2: note = D-Alanine
  SITE 4: note = D-phenylalanine
  SITE 6: note = b-(2-naphthyl) alanine
CXHXRXAC

SEQ ID NO: 157 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
XCXHXRWAC

SEQ ID NO: 158 - protein, 9 aa, synthetic construct
  REGION 1..9: note = Description of Sequence: Synthetic peptide
  SITE 1: note = Nle
  SITE 3: note = D-Alanine
  SITE 5: note = D-phenylalanine
  SITE 7: note = 3-benzothienylalanine
XDXHXRXAK

SEQ ID NO: 159 - protein, 18 aa, synthetic construct
  REGION 1..18: note = Description of Sequence: Synthetic peptide
  SITE 12: note = Nle
  SITE 15: note = D-b-(2-naphthyl) alanine
YGRKKRRQRRRXDHXRWK

SEQ ID NO: 160 - protein, 19 aa, synthetic construct
  REGION 1..19: note = Description of Sequence: Synthetic peptide
  SITE 12: note = 8-amino-3,6-dioxaoctanoic acid
  SITE 13: note = Nle
  SITE 16: note = D-b-(2-naphthyl) alanine
YGRKKRRQRRRXXDHXRWK

SEQ ID NO: 161 - protein, 19 aa, synthetic construct
  REGION 1..19: note = Description of Sequence: Synthetic peptide
  SITE 1: note = Nle
  SITE 4: note = D-b-(2-naphthyl) alanine
  SITE 8: note = bAla
XDHXRWKXYGRKKRRQRRR

SEQ ID NO: 162 - protein, 19 aa, synthetic construct
  REGION 1..19: note = Description of Sequence: Synthetic peptide
  SITE 1: note = Nle
  SITE 4: note = D-b-(2-naphthyl) alanine
  SITE 8: note = bAla
XDHXRWKXYGRKKRRQRRR

SEQ ID NO: 163 - protein, 20 aa, synthetic construct
  REGION 1..20: note = Description of Sequence: Synthetic peptide
  SITE 1: note = Nle
  SITE 4: note = D-b-(2-naphthyl) alanine
  SITE 8: note = 8-amino-3,6-dioxaoctanoic acid
  SITE 9: note = 8-amino-3,6-dioxaoctanoic acid
XDHXRWKXXYGRKKRRQRRR

SEQ ID NO: 164 - protein, 22 aa, synthetic construct
  REGION 1..22: note = Description of Sequence: Synthetic peptide
  SITE 1: note = Nle
  SITE 4: note = D-b-(2-naphthyl) alanine
XDHXRWKPPKDYGRKKRRQRRR

SEQ ID NO: 165 - protein, 23 aa, synthetic construct
  REGION 1..23: note = Description of Sequence: Synthetic peptide
  SITE 4: note = D-b-(2-naphthyl) alanine
CEHXRWGCPPKDYGRKKRRQRRR

SEQ ID NO: 166 - protein, 20 aa, synthetic construct
  REGION 1..20: note = Description of Sequence: Synthetic peptide
  SITE 1: note = Nle
  SITE 4: note = D-b-(2-naphthyl) alanine
  SITE 8: note = bAla
  SITE 9: note = bAla
XDHXRWKXXYGRKKRRQRRR

SEQ ID NO: 167 - protein, 23 aa, synthetic construct
  REGION 1..23: note = Description of Sequence: Synthetic peptide
  SITE 1: note = Nle
  SITE 4: note = D-b-(2-naphthyl) alanine
  SITE 12: note = 8-amino-3,6-dioxaoctanoic acid
XDHXRWKPPKDXYGRKKRRQRRR

SEQ ID NO: 168 - protein, 24 aa, synthetic construct
  REGION 1..24: note = Description of Sequence: Synthetic peptide
  SITE 4: note = D-b-(2-naphthyl) alanine
  SITE 13: note = 8-amino-3,6-dioxaoctanoic acid
CEHXRWGCPPKDXYGRKKRRQRRR

SEQ ID NO: 169 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKKRRQRRR

SEQ ID NO: 170 - protein, 24 aa, synthetic construct
  REGION 1..24: note = Description of Sequence: Synthetic peptide
  SITE 4: note = D-b-(2-naphthyl) alanine
  SITE 13: note = 8-amino-3,6-dioxaoctanoic acid
CEHXRWACPPKDXYGRKKRRQRRR

SEQ ID NO: 171 - protein, 20 aa, synthetic construct
  REGION 1..20: note = Description of Sequence: Synthetic peptide
  SITE 1: note = Nle
  SITE 4: note = D-b-(2-naphthyl) alanine
  SITE 8: note = 8-amino-3,6-dioxaoctanoic acid
  SITE 9: note = 8-amino-3,6-dioxaoctanoic acid
XDHXRWKXXYGRKKRRQRRR

SEQ ID NO: 172 - protein, 24 aa, synthetic construct
  REGION 1..24: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXYGRKKRRQRRR

SEQ ID NO: 173 - protein, 22 aa, synthetic construct
  REGION 1..22: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXRRRRRQRRR

SEQ ID NO: 174 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXGRRRRRQRRR

SEQ ID NO: 175 - protein, 24 aa, synthetic construct
  REGION 1..24: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXYGRRRRRQRRR

SEQ ID NO: 176 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKKRQRRRR

SEQ ID NO: 177 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKKQRRRRR

SEQ ID NO: 178 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKQKRRRRR

SEQ ID NO: 179 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKKRRRRQR

SEQ ID NO: 180 - protein, 24 aa, synthetic construct
  REGION 1..24: note = Description of Sequence: Synthetic peptide
  SITE 4: note = D-b-(2-naphthyl) alanine
  SITE 6: note = 3-benzothienylalanine
  SITE 13: note = bAla
  SITE 15: note = Aib
CEHXRXACPPKDXYXRKKRRQRRR

SEQ ID NO: 181 - protein, 22 aa, synthetic construct
  REGION 1..22: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPRDXRRRRRQRRR

SEQ ID NO: 182 - protein, 22 aa, synthetic construct
  REGION 1..22: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXRRRRRQRRR

SEQ ID NO: 183 - protein, 23 aa, synthetic construct
  REGION 1..23: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXRRRRRRQRRR

SEQ ID NO: 184 - protein, 22 aa, synthetic construct
  REGION 1..22: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPRDXRRRRRQRRR

SEQ ID NO: 185 - protein, 22 aa, synthetic construct
  REGION 1..22: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXRRRRRQRRR

SEQ ID NO: 186 - protein, 23 aa, synthetic construct
  REGION 1..23: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXRRRRRRQRRR

SEQ ID NO: 187 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPRDXRRRRRRQRRR

SEQ ID NO: 188 - protein, 22 aa, synthetic construct
  REGION 1..22: note = Description of Sequence: Synthetic peptide
  SITE 4: note = D-b-(2-naphthyl) alanine
  SITE 6: note = 3-benzothienylalanine
  SITE 13: note = bAla
CEHXRXACPPRDXRRRRRQRRR

SEQ ID NO: 189 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXRRRRRRQRRR

SEQ ID NO: 190 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKKRRRQRR

SEQ ID NO: 191 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRQKKRRRRR

SEQ ID NO: 192 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRKKRRRRRQ

SEQ ID NO: 193 - protein, 24 aa, synthetic construct
  REGION 1..24: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXYGRKKRRQRRR

SEQ ID NO: 194 - protein, 24 aa, synthetic construct
  REGION 1..24: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXYGRKKRRQRRR

SEQ ID NO: 195 - protein, 22 aa, synthetic construct
  REGION 1..22: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXRRKRRQRRR

SEQ ID NO: 196 - protein, 22 aa, synthetic construct
  REGION 1..22: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXRKRRRQRRR

SEQ ID NO: 197 - protein, 22 aa, synthetic construct
  REGION 1..22: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXRRKRRQRRR

SEQ ID NO: 198 - protein, 24 aa, synthetic construct
  REGION 1..24: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXYGRRKRRQRRR

SEQ ID NO: 199 - protein, 23 aa, synthetic construct
  REGION 1..23: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXGRRKRRQRRR

SEQ ID NO: 200 - protein, 23 aa, synthetic construct
  REGION 1..23: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXGRKRRRQRRR

SEQ ID NO: 201 - protein, 24 aa, synthetic construct
  REGION 1..24: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXYGRRKRRQRRR

SEQ ID NO: 202 - protein, 24 aa, synthetic construct
  REGION 1..24: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXYGRKRRRQRRR

SEQ ID NO: 203 - protein, 23 aa, synthetic construct
  REGION 1..23: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXGRRKRRQRRR

SEQ ID NO: 204 - protein, 23 aa, synthetic construct
  REGION 1..23: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXGRKRRRQRRR

SEQ ID NO: 205 - protein, 22 aa, synthetic construct
  REGION 1..22: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXRRKRRQRRR

SEQ ID NO: 206 - protein, 22 aa, synthetic construct
  REGION 1..22: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXRKRRRQRRR

SEQ ID NO: 207 - protein, 24 aa, synthetic construct
  REGION 1..24: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXYGRKRRRQRRR

SEQ ID NO: 208 - protein, 22 aa, synthetic construct
  REGION 1..22: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXRRKRRQRRR

SEQ ID NO: 209 - protein, 22 aa, synthetic construct
  REGION 1..22: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXRKRRRQRRR

SEQ ID NO: 210 - protein, 24 aa, synthetic construct
  REGION 1..24: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXYGRRKRRQRRR

SEQ ID NO: 211 - protein, 24 aa, synthetic construct
  REGION 1..24: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXYGRKRRRQRRR

SEQ ID NO: 212 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXGRRKRRQRRR

SEQ ID NO: 213 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXGRKRRRQRRR

SEQ ID NO: 214 - protein, 22 aa, synthetic construct
  REGION 1..22: note = Description of Sequence: Synthetic peptide
  SITE 4: note = D-b-(2-naphthyl) alanine
  SITE 13: note = bAla
CEHXRWACPPKDXRRRRRQRRR

SEQ ID NO: 215 - protein, 22 aa, synthetic construct
  REGION 1..22: note = Description of Sequence: Synthetic peptide
  SITE 4: note = D-b-(2-naphthyl) alanine
  SITE 13: note = bAla
CEHXRWACPPRDXRRRRRQRRR

SEQ ID NO: 216 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPKDXYGRRRRRQRRR

SEQ ID NO: 217 - protein, 24 aa, synthetic construct
  REGION 1..24: note = Description of Sequence: Synthetic peptide
  SITE 4: note = D-b-(2-naphthyl) alanine
  SITE 13: note = bAla
CEHXRWACPPRDXYGRRRRRQRRR

SEQ ID NO: 218 - protein, 23 aa, synthetic construct
  REGION 1..23: note = Description of Sequence: Synthetic peptide
  SITE 4: note = D-b-(2-naphthyl) alanine
  SITE 13: note = bAla
CEHXRWACPPKDXRRRRRQRRRR

SEQ ID NO: 219 - protein, 23 aa, synthetic construct
  REGION 1..23: note = Description of Sequence: Synthetic peptide
  SITE 4: note = D-b-(2-naphthyl) alanine
  SITE 13: note = bAla
CEHXRWACPPRDXRRRRRQRRRR

SEQ ID NO: 220 - protein, 25 aa, synthetic construct
  REGION 1..25: note = Description of Sequence: Synthetic peptide
  SITE 4: note = D-b-(2-naphthyl) alanine
  SITE 13: note = bAla
CEHXRWACPPKDXYGRRRRRQRRRR

SEQ ID NO: 221 - protein, 25 aa, synthetic construct
  REGION 1..25: note = Description of Sequence: Synthetic peptide
  SITE 4: note = D-b-(2-naphthyl) alanine
  SITE 13: note = bAla
CEHXRWACPPRDXYGRRRRRQRRRR

SEQ ID NO: 222 - protein, 23 aa, synthetic construct
  REGION 1..23: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXRRRRRQRRRR

SEQ ID NO: 223 - protein, 23 aa, synthetic construct
  REGION 1..23: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPRDXRRRRRQRRRR

SEQ ID NO: 224 - protein, 23 aa, synthetic construct
  REGION 1..23: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPRDXRRRRRRQRRR

SEQ ID NO: 225 - protein, 24 aa, synthetic construct
  REGION 1..24: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXYGRRRRRQRRR

SEQ ID NO: 226 - protein, 24 aa, synthetic construct
  REGION 1..24: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPRDXYGRRRRRQRRR

SEQ ID NO: 227 - protein, 25 aa, synthetic construct
  REGION 1..25: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXYGRRRRRQRRRR

SEQ ID NO: 228 - protein, 25 aa, synthetic construct
  REGION 1..25: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPRDXYGRRRRRQRRRR

SEQ ID NO: 229 - protein, 25 aa, synthetic construct
  REGION 1..25: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPKDXYGRRRRRRQRRR

SEQ ID NO: 230 - protein, 25 aa, synthetic construct
  REGION 1..25: note = Description of Sequence: Synthetic peptide
  SITE 4: note = D-b-(2-naphthyl) alanine
  SITE 6: note = b-(1-naphthyl) alanine
  SITE 13: note = bAla
CEHXRXACPPRDXYGRRRRRRQRRR

SEQ ID NO: 231 - protein, 23 aa, synthetic construct
  REGION 1..23: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPRDXRRRRRRQRRR

SEQ ID NO: 232 - protein, 23 aa, synthetic construct
  REGION 1..23: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXRRRRRQRRRR

SEQ ID NO: 233 - protein, 23 aa, synthetic construct
  REGION 1..23: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPRDXRRRRRQRRRR

SEQ ID NO: 234 - protein, 24 aa, synthetic construct
  REGION 1..24: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXYGRRRRRQRRR

SEQ ID NO: 235 - protein, 24 aa, synthetic construct
  REGION 1..24: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPRDXYGRRRRRQRRR

SEQ ID NO: 236 - protein, 25 aa, synthetic construct
  REGION 1..25: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXYGRRRRRRQRRR

SEQ ID NO: 237 - protein, 25 aa, synthetic construct
  REGION 1..25: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPRDXYGRRRRRRQRRR

SEQ ID NO: 238 - protein, 25 aa, synthetic construct
  REGION 1..25: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPKDXYGRRRRRQRRRR

SEQ ID NO: 239 - protein, 25 aa, synthetic construct
  REGION 1..25: note = Description of Sequence: Synthetic peptide
  SITE 4: note = D-b-(2-naphthyl) alanine
  SITE 6: note = b-(2-naphthyl) alanine
  SITE 13: note = bAla
CEHXRXACPPRDXYGRRRRRQRRRR

SEQ ID NO: 240 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXRRRRRQRRRR

SEQ ID NO: 241 - protein, 23 aa, synthetic construct
  REGION 1..23: note = Description of Sequence: Synthetic peptide
  SITE 4: note = D-b-(2-naphthyl) alanine
  SITE 6: note = 3-benzothienylalanine
  SITE 13: note = bAla
CEHXRXACPPRDXRRRRRQRRRR

SEQ ID NO: 242 - protein, 24 aa, synthetic construct
  REGION 1..24: note = Description of Sequence: Synthetic peptide
  SITE 4: note = D-b-(2-naphthyl) alanine
  SITE 6: note = 3-benzothienylalanine
  SITE 13: note = bAla
CEHXRXACPPRDXYGRRRRRQRRR

SEQ ID NO: 243 - protein, 25 aa, synthetic construct
  REGION 1..25: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXYGRRRRRQRRRR

SEQ ID NO: 244 - protein, 25 aa, synthetic construct
  REGION 1..25: note = Description of Sequence: Synthetic peptide
  SITE 4: note = D-b-(2-naphthyl) alanine
  SITE 6: note = 3-benzothienylalanine
  SITE 13: note = bAla
CEHXRXACPPRDXYGRRRRRQRRRR

SEQ ID NO: 245 - protein, 25 aa, synthetic construct
  REGION 1..25: note = Description of Sequence: Synthetic peptide
  SITE 4: note = D-b-(2-naphthyl) alanine
  SITE 6: note = 3-benzothienylalanine
  SITE 13: note = bAla
CEHXRXACPPKDXYGRRRRRRQRRR

SEQ ID NO: 246 - protein, 25 aa, synthetic construct
  REGION 1..25: note = Description of Sequence: Synthetic peptide
  SITE 4: note = D-b-(2-naphthyl) alanine
  SITE 6: note = 3-benzothienylalanine
  SITE 13: note = bAla
CEHXRXACPPRDXYGRRRRRRQRRR

SEQ ID NO: 247 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRKKRRQRRR

SEQ ID NO: 248 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
XCXHXRWCXYGRKKRQRRRR

SEQ ID NO: 249 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXYGRKKRRQRRR

SEQ ID NO: 250 - protein, 18 aa, synthetic construct
  REGION 1..18: note = Description of Sequence: Synthetic peptide
  SITE 1: note = Nle
  SITE 3: note = D-Alanine
  SITE 5: note = D-phenylalanine
  SITE 9: note = bAla
XCXHXRWCXRRRRRQRRR

SEQ ID NO: 251 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = bAla
XCXHXRWCXGRRRRRQRRR

SEQ ID NO: 252 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
XCXHXRWCXYGRRRRRQRRR

SEQ ID NO: 253 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
XCXHXRWCXGRRRRRQRRRR

SEQ ID NO: 254 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
XCXHXRWCXYGRRKRRQRRR

SEQ ID NO: 255 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
XCXHXRWCXYGRKRRRQRRR

SEQ ID NO: 256 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = bAla
XCXHXRWCXGRRKRRQRRR

SEQ ID NO: 257 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = bAla
XCXHXRWCXGRKRRRQRRR

SEQ ID NO: 258 - protein, 18 aa, synthetic construct
  REGION 1..18: note = Description of Sequence: Synthetic peptide
  SITE 1: note = Nle
  SITE 3: note = D-Alanine
  SITE 5: note = D-phenylalanine
  SITE 9: note = bAla
XCXHXRWCXRRKRRQRRR

SEQ ID NO: 259 - protein, 18 aa, synthetic construct
  REGION 1..18: note = Description of Sequence: Synthetic peptide
  SITE 1: note = Nle
  SITE 3: note = D-Alanine
  SITE 5: note = D-phenylalanine
  SITE 9: note = bAla
XCXHXRWCXRKRRRQRRR

SEQ ID NO: 260 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = bAla
  SITE 10: note = bAla
XCXHXRWCXXRRRRRQRRR

SEQ ID NO: 261 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
  SITE 10: note = bAla
XCXHXRWCXXGRRRRRQRRR

SEQ ID NO: 262 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = bAla
  SITE 10: note = bAla
XCXHXRWCXXYGRRRRRQRRR

SEQ ID NO: 263 - protein, 18 aa, synthetic construct
  REGION 1..18: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXRRRRRQRRR

SEQ ID NO: 264 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXGRRRRRQRRR

SEQ ID NO: 265 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXYGRRRRRQRRR

SEQ ID NO: 266 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXRRRRRQRRR

SEQ ID NO: 267 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXGRRRRRQRRR

SEQ ID NO: 268 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRRRRRQRRR

SEQ ID NO: 269 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = bAla
XCXHXRWCXRRRRRQRRRR

SEQ ID NO: 270 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = bAla
XCXHXRWCXYGRRRRRQRRRR

SEQ ID NO: 271 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = bAla
  SITE 10: note = bAla
XCXHXRWCXXRRRRRQRRRR

SEQ ID NO: 272 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = bAla
  SITE 10: note = bAla
XCXHXRWCXXGRRRRRQRRRR

SEQ ID NO: 273 - protein, 22 aa, synthetic construct
  REGION 1..22: note = Description of Sequence: Synthetic peptide
  SITE 1: note = Nle
  SITE 3: note = D-Alanine
  SITE 5: note = D-phenylalanine
  SITE 9: note = bAla
  SITE 10: note = bAla
XCXHXRWCXXYGRRRRRQRRRR

SEQ ID NO: 274 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXRRRRRQRRRR

SEQ ID NO: 275 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXGRRRRRQRRRR

SEQ ID NO: 276 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
XCXHXRWCXYGRRRRRQRRRR

SEQ ID NO: 277 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXRRRRRQRRRR

SEQ ID NO: 278 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXGRRRRRQRRRR

SEQ ID NO: 279 - protein, 22 aa, synthetic construct
  REGION 1..22: note = Description of Sequence: Synthetic peptide
  SITE 1: note = Nle
  SITE 3: note = D-Alanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRRRRRQRRRR

SEQ ID NO: 280 - protein, 20 aa, synthetic construct
  REGION 1..20: note = Description of Sequence: Synthetic peptide
  SITE 1: note = Nle
  SITE 4: note = D-phenylalanine
  SITE 7: note = 4Abu
  SITE 9: note = bAla
XCHXRWXCXYGRRRRRQRRR

SEQ ID NO: 281 - protein, 18 aa, synthetic construct
  REGION 1..18: note = Description of Sequence: Synthetic peptide
  SITE 1: note = Nle
  SITE 4: note = D-phenylalanine
  SITE 7: note = 4Abu
  SITE 9: note = bAla
XCHXRWXCXRRRRRQRRR

SEQ ID NO: 282 - protein, 20 aa, synthetic construct
  REGION 1..20: note = Description of Sequence: Synthetic peptide
  SITE 1: note = Nle
  SITE 4: note = D-phenylalanine
  SITE 9: note = bAla
XDHXRWAKXYGRRRRRQRRR

SEQ ID NO: 283 - protein, 18 aa, synthetic construct
  REGION 1..18: note = Description of Sequence: Synthetic peptide
  SITE 1: note = Nle
  SITE 4: note = D-phenylalanine
  SITE 9: note = bAla
XDHXRWAKXRRRRRQRRR

SEQ ID NO: 284 - protein, 19 aa, synthetic construct
  REGION 1..19: note = Description of Sequence: Synthetic peptide
  SITE 1: note = Nle
  SITE 4: note = D-phenylalanine
  SITE 8: note = bAla
XDHXRWKXYGRRRRRQRRR

SEQ ID NO: 285 - protein, 18 aa, synthetic construct
  REGION 1..18: note = Description of Sequence: Synthetic peptide
  SITE 1: note = Nle
  SITE 4: note = D-phenylalanine
  SITE 8: note = bAla
XDHXRWKXGRRRRRQRRR

SEQ ID NO: 286 - protein, 17 aa, synthetic construct
  REGION 1..17: note = Description of Sequence: Synthetic peptide
  SITE 1: note = Nle
  SITE 4: note = D-phenylalanine
  SITE 8: note = bAla
XDHXRWKXRRRRRQRRR

SEQ ID NO: 287 - protein, 20 aa, synthetic construct
  REGION 1..20: note = Description of Sequence: Synthetic peptide
  SITE 1: note = Nle
  SITE 4: note = D-phenylalanine
  SITE 8: note = bAla
  SITE 9: note = bAla
XDHXRWKXXYGRRRRRQRRR

SEQ ID NO: 288 - protein, 19 aa, synthetic construct
  REGION 1..19: note = Description of Sequence: Synthetic peptide
  SITE 1: note = Nle
  SITE 4: note = D-phenylalanine
  SITE 8: note = bAla
  SITE 9: note = bAla
XDHXRWKXXGRRRRRQRRR

SEQ ID NO: 289 - protein, 18 aa, synthetic construct
  REGION 1..18: note = Description of Sequence: Synthetic peptide
  SITE 1: note = Nle
  SITE 4: note = D-phenylalanine
  SITE 8: note = bAla
  SITE 9: note = bAla
XDHXRWKXXRRRRRQRRR

SEQ ID NO: 290 - protein, 19 aa, synthetic construct
  REGION 1..19: note = Description of Sequence: Synthetic peptide
  SITE 1: note = Nle
  SITE 4: note = D-phenylalanine
  SITE 8: note = 8-amino-3,6-dioxaoctanoic acid
XDHXRWKXYGRRRRRQRRR

SEQ ID NO: 291 - protein, 18 aa, synthetic construct
  REGION 1..18: note = Description of Sequence: Synthetic peptide
  SITE 1: note = Nle
  SITE 4: note = D-phenylalanine
  SITE 8: note = 8-amino-3,6-dioxaoctanoic acid
XDHXRWKXGRRRRRQRRR

SEQ ID NO: 292 - protein, 17 aa, synthetic construct
  REGION 1..17: note = Description of Sequence: Synthetic peptide
  SITE 1: note = Nle
  SITE 4: note = D-phenylalanine
  SITE 8: note = 8-amino-3,6-dioxaoctanoic acid
XDHXRWKXRRRRRQRRR

SEQ ID NO: 293 - protein, 20 aa, synthetic construct
  REGION 1..20: note = Description of Sequence: Synthetic peptide
  SITE 1: note = Nle
  SITE 4: note = D-phenylalanine
  SITE 8: note = 8-amino-3,6-dioxaoctanoic acid
  SITE 9: note = 8-amino-3,6-dioxaoctanoic acid
XDHXRWKXXYGRRRRRQRRR

SEQ ID NO: 294 - protein, 19 aa, synthetic construct
  REGION 1..19: note = Description of Sequence: Synthetic peptide
  SITE 1: note = Nle
  SITE 4: note = D-phenylalanine
  SITE 8: note = 8-amino-3,6-dioxaoctanoic acid
  SITE 9: note = 8-amino-3,6-dioxaoctanoic acid
XDHXRWKXXGRRRRRQRRR

SEQ ID NO: 295 - protein, 18 aa, synthetic construct
  REGION 1..18: note = Description of Sequence: Synthetic peptide
  SITE 1: note = Nle
  SITE 4: note = D-phenylalanine
  SITE 8: note = 8-amino-3,6-dioxaoctanoic acid
  SITE 9: note = 8-amino-3,6-dioxaoctanoic acid
XDHXRWKXXRRRRRQRRR

SEQ ID NO: 296 - protein, 20 aa, synthetic construct
  REGION 1..20: note = Description of Sequence: Synthetic peptide
  SITE 1: note = Nle
  SITE 4: note = D-phenylalanine
  SITE 8: note = bAla
XDHXRWKXYGRRRRRQRRRR

SEQ ID NO: 297 - protein, 19 aa, synthetic construct
  REGION 1..19: note = Description of Sequence: Synthetic peptide
  SITE 1: note = Nle
  SITE 4: note = D-phenylalanine
  SITE 8: note = bAla
XDHXRWKXGRRRRRQRRRR

SEQ ID NO: 298 - protein, 18 aa, synthetic construct
  REGION 1..18: note = Description of Sequence: Synthetic peptide
  SITE 1: note = Nle
  SITE 4: note = D-phenylalanine
  SITE 8: note = bAla
XDHXRWKXRRRRRQRRRR

SEQ ID NO: 299 - protein, 21 aa, synthetic construct
  REGION 1..21: note = Description of Sequence: Synthetic peptide
  SITE 1: note = Nle
  SITE 4: note = D-phenylalanine
  SITE 8: note = bAla
  SITE 9: note = bAla
XDHXRWKXXYGRRRRRQRRRR

SEQ ID NO: 300 - protein, 20 aa, synthetic construct
  REGION 1..20: note = Description of Sequence: Synthetic peptide
  SITE 1: note = Nle
  SITE 4: note = D-phenylalanine
  SITE 8: note = bAla
  SITE 9: note = bAla
XDHXRWKXXGRRRRRQRRRR

SEQ ID NO: 301 - protein, 19 aa, synthetic construct
  REGION 1..19: note = Description of Sequence: Synthetic peptide
  SITE 1: note = Nle
  SITE 4: note = D-phenylalanine
  SITE 8: note = bAla
  SITE 9: note = bAla
XDHXRWKXXRRRRRQRRRR

SEQ ID NO: 302 - protein, 20 aa, synthetic construct
  REGION 1..20: note = Description of Sequence: Synthetic peptide
  SITE 1: note = Nle
  SITE 4: note = D-phenylalanine
  SITE 8: note = 8-amino-3,6-dioxaoctanoic acid
XDHXRWKXYGRRRRRQRRRR

SEQ ID NO: 303 - protein, 19 aa, synthetic construct
  REGION 1..19: note = Description of Sequence: Synthetic peptide
  SITE 1: note = Nle
  SITE 4: note = D-phenylalanine
  SITE 8: note = 8-amino-3,6-dioxaoctanoic acid
XDHXRWKXGRRRRRQRRRR

SEQ ID NO: 304 - protein, 18 aa, synthetic construct
  REGION 1..18: note = Description of Sequence: Synthetic peptide
  SITE 1: note = Nle
  SITE 4: note = D-phenylalanine
  SITE 8: note = 8-amino-3,6-dioxaoctanoic acid
XDHXRWKXRRRRRQRRRR

SEQ ID NO: 305 - protein, 21 aa, synthetic construct
  REGION 1..21: note = Description of Sequence: Synthetic peptide
  SITE 1: note = Nle
  SITE 4: note = D-phenylalanine
  SITE 8: note = 8-amino-3,6-dioxaoctanoic acid
  SITE 9: note = 8-amino-3,6-dioxaoctanoic acid
XDHXRWKXXYGRRRRRQRRRR

SEQ ID NO: 306 - protein, 20 aa, synthetic construct
  REGION 1..20: note = Description of Sequence: Synthetic peptide
  SITE 1: note = Nle
  SITE 4: note = D-phenylalanine
  SITE 8: note = 8-amino-3,6-dioxaoctanoic acid
  SITE 9: note = 8-amino-3,6-dioxaoctanoic acid
XDHXRWKXXGRRRRRQRRRR

SEQ ID NO: 307 - protein, 19 aa, synthetic construct
  REGION 1..19: note = Description of Sequence: Synthetic peptide
  SITE 1: note = Nle
  SITE 4: note = D-phenylalanine
  SITE 8: note = 8-amino-3,6-dioxaoctanoic acid
  SITE 9: note = 8-amino-3,6-dioxaoctanoic acid
XDHXRWKXXRRRRRQRRRR

SEQ ID NO: 308 - protein, 20 aa, synthetic construct
  REGION 1..20: note = Description of Sequence: Synthetic peptide
  SITE 1: note = Nle
  SITE 4: note = D-phenylalanine
  SITE 7: note = bAla
  SITE 9: note = bAla
XDHXRWXKXYGRRRRRQRRR

SEQ ID NO: 309 - protein, 18 aa, synthetic construct
  REGION 1..18: note = Description of Sequence: Synthetic peptide
  SITE 1: note = Nle
  SITE 4: note = D-phenylalanine
  SITE 7: note = bAla
  SITE 9: note = bAla
XDHXRWXKXRRRRRQRRR

SEQ ID NO: 310 - protein, 20 aa, synthetic construct
  REGION 1..20: note = Description of Sequence: Synthetic peptide
  SITE 1: note = Nle
  SITE 4: note = D-phenylalanine
  SITE 7: note = 6-aminohexanoic acid
  SITE 9: note = bAla
XCHXRWXCXYGRRRRRQRRR

SEQ ID NO: 311 - protein, 18 aa, synthetic construct
  REGION 1..18: note = Description of Sequence: Synthetic peptide
  SITE 1: note = Nle
  SITE 4: note = D-phenylalanine
  SITE 7: note = 6-aminohexanoic acid
  SITE 9: note = bAla
XCHXRWXCXRRRRRQRRR

SEQ ID NO: 312 - protein, 21 aa, synthetic construct
  REGION 1..21: note = Description of Sequence: Synthetic peptide
  SITE 1: note = D-phenylalanine
  SITE 4: note = D-phenylalanine
  SITE 7: note = bAla
  SITE 8: note = D-cysteine
  SITE 10: note = bAla
XCHXRWXXTXYGRRRRRQRRR

SEQ ID NO: 313 - protein, 19 aa, synthetic construct
  REGION 1..19: note = Description of Sequence: Synthetic peptide
  SITE 1: note = D-phenylalanine
  SITE 4: note = D-phenylalanine
  SITE 7: note = bAla
  SITE 8: note = D-cysteine
  SITE 10: note = bAla
XCHXRWXXTXRRRRRQRRR

SEQ ID NO: 314 - protein, 20 aa, synthetic construct
  REGION 1..20: note = Description of Sequence: Synthetic peptide
  SITE 1: note = Nle
  SITE 4: note = D-phenylalanine
  SITE 7: note = 5-aminopentanoic acid
  SITE 9: note = bAla
XCHXRWXCXYGRRRRRQRRR

SEQ ID NO: 315 - protein, 18 aa, synthetic construct
  REGION 1..18: note = Description of Sequence: Synthetic peptide
  SITE 1: note = Nle
  SITE 4: note = D-phenylalanine
  SITE 7: note = 5-aminopentanoic acid
  SITE 9: note = bAla
XCHXRWXCXRRRRRQRRR

SEQ ID NO: 316 - protein, 20 aa, synthetic construct
  REGION 1..20: note = Description of Sequence: Synthetic peptide
  SITE 1: note = b-cyclohexylalanine
  SITE 4: note = D-phenylalanine
  SITE 7: note = 4Abu
  SITE 9: note = bAla
XDHXRWXKXYGRRRRRQRRR

SEQ ID NO: 317 - protein, 18 aa, synthetic construct
  REGION 1..18: note = Description of Sequence: Synthetic peptide
  SITE 1: note = b-cyclohexylalanine
  SITE 4: note = D-phenylalanine
  SITE 7: note = 4Abu
  SITE 9: note = bAla
XDHXRWXKXRRRRRQRRR

SEQ ID NO: 318 - protein, 20 aa, synthetic construct
  REGION 1..20: note = Description of Sequence: Synthetic peptide
  SITE 1: note = Nle
  SITE 4: note = D-phenylalanine
  SITE 7: note = 4Abu
  SITE 9: note = bAla
XDHXRWXKXYGRRRRRQRRR

SEQ ID NO: 319 - protein, 18 aa, synthetic construct
  REGION 1..18: note = Description of Sequence: Synthetic peptide
  SITE 1: note = Nle
  SITE 4: note = D-phenylalanine
  SITE 7: note = 4Abu
  SITE 9: note = bAla
XDHXRWXKXRRRRRQRRR

SEQ ID NO: 320 - protein, 20 aa, synthetic construct
  REGION 1..20: note = Description of Sequence: Synthetic peptide
  SITE 1: note = Cyclohexylglycine
  SITE 4: note = D-phenylalanine
  SITE 7: note = 4Abu
  SITE 9: note = bAla
XDHXRWXKXYGRRRRRQRRR

SEQ ID NO: 321 - protein, 18 aa, synthetic construct
  REGION 1..18: note = Description of Sequence: Synthetic peptide
  SITE 1: note = Cyclohexylglycine
  SITE 4: note = D-phenylalanine
  SITE 7: note = 4Abu
  SITE 9: note = bAla
XDHXRWXKXRRRRRQRRR

SEQ ID NO: 322 - protein, 20 aa, synthetic construct
  REGION 1..20: note = Description of Sequence: Synthetic peptide
  SITE 1: note = homo-cyclohexylalanine
  SITE 4: note = D-phenylalanine
  SITE 7: note = 4Abu
  SITE 9: note = bAla
XDHXRWXKXYGRRRRRQRRR

SEQ ID NO: 323 - protein, 18 aa, synthetic construct
  REGION 1..18: note = Description of Sequence: Synthetic peptide
  SITE 1: note = homo-cyclohexylalanine
  SITE 4: note = D-phenylalanine
  SITE 7: note = 4Abu
  SITE 9: note = bAla
XDHXRWXKXRRRRRQRRR

SEQ ID NO: 324 - protein, 21 aa, synthetic construct
  REGION 1..21: note = Description of Sequence: Synthetic peptide
  SITE 1: note = homo-cyclohexylalanine
  SITE 4: note = D-phenylalanine
  SITE 7: note = 4Abu
  SITE 9: note = bAla
  SITE 10: note = bAla
XDHXRWXKXXYGRRRRRQRRR

SEQ ID NO: 325 - protein, 19 aa, synthetic construct
  REGION 1..19: note = Description of Sequence: Synthetic peptide
  SITE 1: note = homo-cyclohexylalanine
  SITE 4: note = D-phenylalanine
  SITE 7: note = 4Abu
  SITE 9: note = bAla
  SITE 10: note = bAla
XDHXRWXKXXRRRRRQRRR

SEQ ID NO: 326 - protein, 20 aa, synthetic construct
  REGION 1..20: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
XDHXRWXKXYGRRRRRQRRR

SEQ ID NO: 327 - protein, 18 aa, synthetic construct
  REGION 1..18: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
XDHXRWXKXRRRRRQRRR

SEQ ID NO: 328 - protein, 21 aa, synthetic construct
  REGION 1..21: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XDHXRWXKXXYGRRRRRQRRR

SEQ ID NO: 329 - protein, 19 aa, synthetic construct
  REGION 1..19: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XDHXRWXKXXRRRRRQRRR

SEQ ID NO: 330 - protein, 21 aa, synthetic construct
  REGION 1..21: note = Description of Sequence: Synthetic peptide
  SITE 1: note = homo-cyclohexylalanine
  SITE 4: note = D-phenylalanine
  SITE 7: note = 4Abu
  SITE 9: note = bAla
XDHXRWXKXYGRRRRRQRRRR

SEQ ID NO: 331 - protein, 19 aa, synthetic construct
  REGION 1..19: note = Description of Sequence: Synthetic peptide
  SITE 1: note = homo-cyclohexylalanine
  SITE 4: note = D-phenylalanine
  SITE 7: note = 4Abu
  SITE 9: note = bAla
XDHXRWXKXRRRRRQRRRR

SEQ ID NO: 332 - protein, 22 aa, synthetic construct
  REGION 1..22: note = Description of Sequence: Synthetic peptide
  SITE 1: note = homo-cyclohexylalanine
  SITE 4: note = D-phenylalanine
  SITE 7: note = 4Abu
  SITE 9: note = bAla
  SITE 10: note = bAla
XDHXRWXKXXYGRRRRRQRRRR

SEQ ID NO: 333 - protein, 20 aa, synthetic construct
  REGION 1..20: note = Description of Sequence: Synthetic peptide
  SITE 1: note = homo-cyclohexylalanine
  SITE 4: note = D-phenylalanine
  SITE 7: note = 4Abu
  SITE 9: note = bAla
  SITE 10: note = bAla
XDHXRWXKXXRRRRRQRRRR

SEQ ID NO: 334 - protein, 21 aa, synthetic construct
  REGION 1..21: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
XDHXRWXKXYGRRRRRQRRRR

SEQ ID NO: 335 - protein, 19 aa, synthetic construct
  REGION 1..19: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
XDHXRWXKXRRRRRQRRRR

SEQ ID NO: 336 - protein, 22 aa, synthetic construct
  REGION 1..22: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XDHXRWXKXXYGRRRRRQRRRR

SEQ ID NO: 337 - protein, 20 aa, synthetic construct
  REGION 1..20: note = Description of Sequence: Synthetic peptide
  SITE 1: note = homo-cyclohexylalanine
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XDHXRWXKXXRRRRRQRRRR

SEQ ID NO: 338 - protein, 20 aa, synthetic construct
  REGION 1..20: note = Description of Sequence: Synthetic peptide
  SITE 1: note = D-Cyclohexylglycine
  SITE 4: note = D-phenylalanine
  SITE 7: note = 4Abu
  SITE 9: note = bAla
XDHXRWXKXYGRRRRRQRRR

SEQ ID NO: 339 - protein, 18 aa, synthetic construct
  REGION 1..18: note = Description of Sequence: Synthetic peptide
  SITE 1: note = D-Cyclohexylglycine
  SITE 4: note = D-phenylalanine
  SITE 7: note = 4Abu
  SITE 9: note = bAla
XDHXRWXKXRRRRRQRRR

SEQ ID NO: 340 - protein, 20 aa, synthetic construct
  REGION 1..20: note = Description of Sequence: Synthetic peptide
  SITE 4: note = D-phenylalanine
  SITE 7: note = 4Abu
  SITE 9: note = bAla
FDHXRWXKXYGRRRRRQRRR

SEQ ID NO: 341 - protein, 18 aa, synthetic construct
  REGION 1..18: note = Description of Sequence: Synthetic peptide
  SITE 4: note = D-phenylalanine
  SITE 7: note = 4Abu
  SITE 9: note = bAla
FDHXRWXKXRRRRRQRRR

SEQ ID NO: 342 - protein, 20 aa, synthetic construct
  REGION 1..20: note = Description of Sequence: Synthetic peptide
  SITE 1: note = Nle
  SITE 4: note = D-phenylalanine
  SITE 6: note = D-Tryptophan
  SITE 7: note = 5-aminopentanoic acid
  SITE 9: note = bAla
XCHXRXXCXYGRRRRRQRRR

SEQ ID NO: 343 - protein, 18 aa, synthetic construct
  REGION 1..18: note = Description of Sequence: Synthetic peptide
  SITE 1: note = Nle
  SITE 4: note = D-phenylalanine
  SITE 6: note = D-Tryptophan
  SITE 7: note = 5-aminopentanoic acid
  SITE 9: note = bAla
XCHXRXXCXRRRRRQRRR

SEQ ID NO: 344 - protein, 20 aa, synthetic construct
  REGION 1..20: note = Description of Sequence: Synthetic peptide
  SITE 1: note = Nle
  SITE 4: note = D-phenylalanine
  SITE 6: note = D-Tryptophan
  SITE 7: note = 6-aminohexanoic acid
  SITE 9: note = bAla
XCHXRXXCXYGRRRRRQRRR

SEQ ID NO: 345 - protein, 18 aa, synthetic construct
  REGION 1..18: note = Description of Sequence: Synthetic peptide
  SITE 1: note = Nle
  SITE 4: note = D-phenylalanine
  SITE 6: note = D-Tryptophan
  SITE 7: note = 6-aminohexanoic acid
  SITE 9: note = bAla
XCHXRXXCXRRRRRQRRR

SEQ ID NO: 346 - protein, 20 aa, synthetic construct
  REGION 1..20: note = Description of Sequence: Synthetic peptide
  SITE 1: note = Nle
  SITE 4: note = D-phenylalanine
  SITE 6: note = D-Tryptophan
  SITE 7: note = bAla
  SITE 9: note = bAla
XCHXRXXCXYGRRRRRQRRR

SEQ ID NO: 347 - protein, 18 aa, synthetic construct
  REGION 1..18: note = Description of Sequence: Synthetic peptide
  SITE 1: note = Nle
  SITE 4: note = D-phenylalanine
  SITE 6: note = D-Tryptophan
  SITE 7: note = bAla
  SITE 9: note = bAla
XCHXRXXCXRRRRRQRRR

SEQ ID NO: 348 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = bAla
XCXHXRWXXYGRRRRRQRRR

SEQ ID NO: 349 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = bAla
XCXHXRWXXGRRRRRQRRR

SEQ ID NO: 350 - protein, 18 aa, synthetic construct
  REGION 1..18: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = bAla
XCXHXRWXXRRRRRQRRR

SEQ ID NO: 351 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = bAla
  SITE 10: note = bAla
XCXHXRWXXXYGRRRRRQRRR

SEQ ID NO: 352 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = bAla
  SITE 10: note = bAla
XCXHXRWXXXGRRRRRQRRR

SEQ ID NO: 353 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = bAla
  SITE 10: note = bAla
XCXHXRWXXXRRRRRQRRR

SEQ ID NO: 354 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = 8-amino-3,6-dioxaoctanoic acid
XCXHXRWXXYGRRRRRQRRR

SEQ ID NO: 355 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = 8-amino-3,6-dioxaoctanoic acid
XCXHXRWXXGRRRRRQRRR

SEQ ID NO: 356 - protein, 18 aa, synthetic construct
  REGION 1..18: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = 8-amino-3,6-dioxaoctanoic acid
XCXHXRWXXRRRRRQRRR

SEQ ID NO: 357 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = 8-amino-3,6-dioxaoctanoic acid
  SITE 10: note = 8-amino-3,6-dioxaoctanoic acid
XCXHXRWXXXYGRRRRRQRRR

SEQ ID NO: 358 - protein, 20 aa, synthetic construct
  REGION 1..20: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = 8-amino-3,6-dioxaoctanoic acid
  SITE 10: note = 8-amino-3,6-dioxaoctanoic acid
XCXHXRWXXXGRRRRRQRRR

SEQ ID NO: 359 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 8: note = Penicillamine
  SITE 9: note = 8-amino-3,6-dioxaoctanoic acid
  SITE 10: note = 8-amino-3,6-dioxaoctanoic acid
XCXHXRWXXXRRRRRQRRR

SEQ ID NO: 360 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
XCHXRWXXXYGRRRRRQRRR

SEQ ID NO: 361 - protein, 18 aa, synthetic construct
  REGION 1..18: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
XCHXRWXXXRRRRRQRRR

SEQ ID NO: 362 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
XCHXRWXXXGRRRRRQRRR

SEQ ID NO: 363 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
XCHXRWXXXRRRRRQRRRR

SEQ ID NO: 364 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
  SITE 10: note = bAla
XCHXRWXXXXYGRRRRRQRRR

SEQ ID NO: 365 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
  SITE 10: note = bAla
XCHXRWXXXXRRRRRQRRR

SEQ ID NO: 366 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
  SITE 10: note = bAla
XCHXRWXXXXGRRRRRQRRR

SEQ ID NO: 367 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = bAla
  SITE 10: note = bAla
XCHXRWXXXXRRRRRQRRRR

SEQ ID NO: 368 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
XCHXRWXXXYGRRRRRQRRR

SEQ ID NO: 369 - protein, 18 aa, synthetic construct
  REGION 1..18: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
XCHXRWXXXRRRRRQRRR

SEQ ID NO: 370 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
XCHXRWXXXGRRRRRQRRR

SEQ ID NO: 371 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
XCHXRWXXXRRRRRQRRRR

SEQ ID NO: 372 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
  SITE 10: note = 8-amino-3,6-dioxaoctanoic acid
XCHXRWXXXXYGRRRRRQRRR

SEQ ID NO: 373 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
  SITE 10: note = 8-amino-3,6-dioxaoctanoic acid
XCHXRWXXXXRRRRRQRRR

SEQ ID NO: 374 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
  SITE 10: note = 8-amino-3,6-dioxaoctanoic acid
XCHXRWXXXXGRRRRRQRRR

SEQ ID NO: 375 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 9: note = 8-amino-3,6-dioxaoctanoic acid
  SITE 10: note = 8-amino-3,6-dioxaoctanoic acid
XCHXRWXXXXRRRRRQRRRR

SEQ ID NO: 376 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
XCHXRWXXTXYGRRRRRQRRR

SEQ ID NO: 377 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
XCHXRWXXTXRRRRRQRRR

SEQ ID NO: 378 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
  SITE 11: note = bAla
XCHXRWXXTXXYGRRRRRQRRR

SEQ ID NO: 379 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
  SITE 11: note = bAla
XCHXRWXXTXXRRRRRQRRR

SEQ ID NO: 380 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
XCHXRWXXTXYGRRRRRQRRR

SEQ ID NO: 381 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
XCHXRWXXTXRRRRRQRRR

SEQ ID NO: 382 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
  SITE 11: note = 8-amino-3,6-dioxaoctanoic acid
XCHXRWXXTXXYGRRRRRQRRR

SEQ ID NO: 383 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
XCHXRWXXTXYGRRRRRQRRRR

SEQ ID NO: 384 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
XCHXRWXXTXRRRRRQRRRR

SEQ ID NO: 385 - protein, 23 aa, synthetic construct
  REGION 1..23: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
  SITE 11: note = bAla
XCHXRWXXTXXYGRRRRRQRRRR

SEQ ID NO: 386 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = bAla
  SITE 11: note = bAla
XCHXRWXXTXXRRRRRQRRRR

SEQ ID NO: 387 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
XCHXRWXXTXYGRRRRRQRRRR

SEQ ID NO: 388 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
XCHXRWXXTXRRRRRQRRRR

SEQ ID NO: 389 - protein, 23 aa, synthetic construct
  REGION 1..23: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
  SITE 11: note = 8-amino-3,6-dioxaoctanoic acid
XCHXRWXXTXXYGRRRRRQRRRR

SEQ ID NO: 390 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 7: note = bAla
  SITE 8: note = D-cysteine
  SITE 10: note = 8-amino-3,6-dioxaoctanoic acid
  SITE 11: note = 8-amino-3,6-dioxaoctanoic acid
XCHXRWXXTXXRRRRRQRRRR

SEQ ID NO: 391 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = bAla
XCHXRXXXTXYGRRRRRQRRR

SEQ ID NO: 392 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = bAla
XCHXRXXXTXYGRRRRRQRRRR

SEQ ID NO: 393 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = bAla
XCHXRXXXTXRRRRRQRRR

SEQ ID NO: 394 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = bAla
  SITE 11: note = bAla
XCHXRXXXTXXYGRRRRRQRRR

SEQ ID NO: 395 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = bAla
  SITE 11: note = bAla
XCHXRXXXTXXRRRRRQRRR

SEQ ID NO: 396 - protein, 21 aa, synthetic construct
  REGION 1..21: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = 8-amino-3,6-dioxaoctanoic acid
XCHXRXXXTXYGRRRRRQRRR

SEQ ID NO: 397 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = 8-amino-3,6-dioxaoctanoic acid
XCHXRXXXTXYGRRRRRQRRRR

SEQ ID NO: 398 - protein, 19 aa, synthetic construct
  REGION 1..19: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = 8-amino-3,6-dioxaoctanoic acid
XCHXRXXXTXRRRRRQRRR

SEQ ID NO: 399 - protein, 22 aa, synthetic construct
  REGION 1..22: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = 8-amino-3,6-dioxaoctanoic acid
  SITE 11: note = 8-amino-3,6-dioxaoctanoic acid
XCHXRXXXTXXYGRRRRRQRRR

SEQ ID NO: 400 - protein, 20 aa, synthetic construct
  REGION 1..20: note = Description of Sequence: Synthetic peptide
  SITE 1: note = D-phenylalanine
  SITE 4: note = D-Ethyl-Tyrosine
  SITE 6: note = 4,4 -biphenylalanine
  SITE 7: note = bAla
  SITE 8: note = D-cysteine
  SITE 10: note = 8-amino-3,6-dioxaoctanoic acid
  SITE 11: note = 8-amino-3,6-dioxaoctanoic acid
XCHXRXXXTXXRRRRRQRRR

SEQ ID NO: 401 - protein, 21 aa, synthetic construct
  REGION 1..21: note = Description of Sequence: Synthetic peptide
  SITE 1: note = Nle
  SITE 3: note = D-Alanine
  SITE 5: note = D-phenylalanine
  SITE 10: note = bAla
XCXHXRWGCXYGRRRRRQRRR

SEQ ID NO: 402 - protein, 19 aa, synthetic construct
  REGION 1..19: note = Description of Sequence: Synthetic peptide
  SITE 1: note = Nle
  SITE 3: note = D-Alanine
  SITE 5: note = D-phenylalanine
  SITE 10: note = bAla
XCXHXRWGCXRRRRRQRRR

SEQ ID NO: 403 - protein, 20 aa, synthetic construct
  REGION 1..20: note = Description of Sequence: Synthetic peptide
  SITE 1: note = Nle
  SITE 4: note = D-phenylalanine
  SITE 7: note = 5-aminopentanoic acid
  SITE 9: note = bAla
XCHXRWXCXYGRRRRRQRRR

SEQ ID NO: 404 - protein, 18 aa, synthetic construct
  REGION 1..18: note = Description of Sequence: Synthetic peptide
  SITE 1: note = Nle
  SITE 4: note = D-phenylalanine
  SITE 7: note = 5-aminopentanoic acid
  SITE 9: note = bAla
XCHXRWXCXRRRRRQRRR

SEQ ID NO: 405 - protein, 21 aa, synthetic construct
  REGION 1..21: note = Description of Sequence: Synthetic peptide
  SITE 1: note = Nle
  SITE 4: note = D-phenylalanine
  SITE 7: note = 5-aminopentanoic acid
  SITE 9: note = bAla
  SITE 10: note = bAla
XCHXRWXCXXYGRRRRRQRRR

SEQ ID NO: 406 - protein, 19 aa, synthetic construct
  REGION 1..19: note = Description of Sequence: Synthetic peptide
  SITE 1: note = Nle
  SITE 4: note = D-phenylalanine
  SITE 7: note = 5-aminopentanoic acid
  SITE 9: note = bAla
  SITE 10: note = bAla
XCHXRWXCXXRRRRRQRRR

SEQ ID NO: 407 - protein, 21 aa, synthetic construct
  REGION 1..21: note = Description of Sequence: Synthetic peptide
  SITE 1: note = Nle
  SITE 4: note = D-phenylalanine
  SITE 7: note = 5-aminopentanoic acid
  SITE 9: note = bAla
XCHXRWXCXYGRRRRRQRRRR

SEQ ID NO: 408 - protein, 19 aa, synthetic construct
  REGION 1..19: note = Description of Sequence: Synthetic peptide
  SITE 1: note = Nle
  SITE 4: note = D-phenylalanine
  SITE 7: note = 5-aminopentanoic acid
  SITE 9: note = bAla
XCHXRWXCXRRRRRQRRRR

SEQ ID NO: 409 - protein, 22 aa, synthetic construct
  REGION 1..22: note = Description of Sequence: Synthetic peptide
  SITE 1: note = Nle
  SITE 4: note = D-phenylalanine
  SITE 7: note = 5-aminopentanoic acid
  SITE 9: note = bAla
  SITE 10: note = bAla
XCHXRWXCXXYGRRRRRQRRRR

SEQ ID NO: 410 - protein, 20 aa, synthetic construct
  REGION 1..20: note = Description of Sequence: Synthetic peptide
  SITE 1: note = Nle
  SITE 4: note = D-phenylalanine
  SITE 7: note = 5-aminopentanoic acid
  SITE 9: note = bAla
  SITE 10: note = bAla
XCHXRWXCXXRRRRRQRRRR

SEQ ID NO: 411 - protein, 20 aa, synthetic construct
  REGION 1..20: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
XCHXRWXCXYGRRRRRQRRR

SEQ ID NO: 412 - protein, 18 aa, synthetic construct
  REGION 1..18: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
XCHXRWXCXRRRRRQRRR

SEQ ID NO: 413 - protein, 21 aa, synthetic construct
  REGION 1..21: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
  SITE 10: note = 8-amino-3,6-dioxaoctanoic acid
XCHXRWXCXXYGRRRRRQRRR

SEQ ID NO: 414 - protein, 19 aa, synthetic construct
  REGION 1..19: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
  SITE 10: note = 8-amino-3,6-dioxaoctanoic acid
XCHXRWXCXXRRRRRQRRR

SEQ ID NO: 415 - protein, 21 aa, synthetic construct
  REGION 1..21: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
XCHXRWXCXYGRRRRRQRRRR

SEQ ID NO: 416 - protein, 19 aa, synthetic construct
  REGION 1..19: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
XCHXRWXCXRRRRRQRRRR

SEQ ID NO: 417 - protein, 22 aa, synthetic construct
  REGION 1..22: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
  SITE 10: note = 8-amino-3,6-dioxaoctanoic acid
XCHXRWXCXXYGRRRRRQRRRR

SEQ ID NO: 418 - protein, 20 aa, synthetic construct
  REGION 1..20: note = Description of Sequence: Synthetic peptide
  SITE 1: note = Nle
  SITE 4: note = D-phenylalanine
  SITE 7: note = 5-aminopentanoic acid
  SITE 9: note = 8-amino-3,6-dioxaoctanoic acid
  SITE 10: note = 8-amino-3,6-dioxaoctanoic acid
XCHXRWXCXXRRRRRQRRRR

SEQ ID NO: 419 - protein, 20 aa, synthetic construct
  REGION 1..20: note = Description of Sequence: Synthetic peptide
  SITE 1: note = Nle
  SITE 3: note = D-leucine
  SITE 5: note = D-phenylalanine
  SITE 9: note = bAla
XCXHXRWCXYGRRRRRQRRR

SEQ ID NO: 420 - protein, 18 aa, synthetic construct
  REGION 1..18: note = Description of Sequence: Synthetic peptide
  SITE 1: note = Nle
  SITE 3: note = D-leucine
  SITE 5: note = D-phenylalanine
  SITE 9: note = bAla
XCXHXRWCXRRRRRQRRR

SEQ ID NO: 421 - protein, 21 aa, synthetic construct
  REGION 1..21: note = Description of Sequence: Synthetic peptide
  SITE 1: note = Nle
  SITE 3: note = D-leucine
  SITE 5: note = D-phenylalanine
  SITE 9: note = bAla
  SITE 10: note = bAla
XCXHXRWCXXYGRRRRRQRRR

SEQ ID NO: 422 - protein, 19 aa, synthetic construct
  REGION 1..19: note = Description of Sequence: Synthetic peptide
  SITE 1: note = Nle
  SITE 3: note = D-leucine
  SITE 5: note = D-phenylalanine
  SITE 9: note = bAla
  SITE 10: note = bAla
XCXHXRWCXXRRRRRQRRR

SEQ ID NO: 423 - protein, 20 aa, synthetic construct
  REGION 1..20: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
XCXHXRWCXYGRRRRRQRRR

SEQ ID NO: 424 - protein, 18 aa, synthetic construct
  REGION 1..18: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
XCXHXRWCXRRRRRQRRR

SEQ ID NO: 425 - protein, 21 aa, synthetic construct
  REGION 1..21: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRRRRRQRRR

SEQ ID NO: 426 - protein, 19 aa, synthetic construct
  REGION 1..19: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXRRRRRQRRR

SEQ ID NO: 427 - protein, 21 aa, synthetic construct
  REGION 1..21: note = Description of Sequence: Synthetic peptide
  SITE 1: note = Nle
  SITE 3: note = D-leucine
  SITE 5: note = D-phenylalanine
  SITE 9: note = bAla
XCXHXRWCXYGRRRRRQRRRR

SEQ ID NO: 428 - protein, 19 aa, synthetic construct
  REGION 1..19: note = Description of Sequence: Synthetic peptide
  SITE 1: note = Nle
  SITE 3: note = D-leucine
  SITE 5: note = D-phenylalanine
  SITE 9: note = bAla
XCXHXRWCXRRRRRQRRRR

SEQ ID NO: 429 - protein, 22 aa, synthetic construct
  REGION 1..22: note = Description of Sequence: Synthetic peptide
  SITE 1: note = Nle
  SITE 3: note = D-leucine
  SITE 5: note = D-phenylalanine
  SITE 9: note = bAla
  SITE 10: note = bAla
XCXHXRWCXXYGRRRRRQRRRR

SEQ ID NO: 430 - protein, 20 aa, synthetic construct
  REGION 1..20: note = Description of Sequence: Synthetic peptide
  SITE 1: note = Nle
  SITE 3: note = D-leucine
  SITE 5: note = D-phenylalanine
  SITE 9: note = bAla
  SITE 10: note = bAla
XCXHXRWCXXRRRRRQRRRR

SEQ ID NO: 431 - protein, 21 aa, synthetic construct
  REGION 1..21: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
XCXHXRWCXYGRRRRRQRRRR

SEQ ID NO: 432 - protein, 19 aa, synthetic construct
  REGION 1..19: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
XCXHXRWCXRRRRRQRRRR

SEQ ID NO: 433 - protein, 22 aa, synthetic construct
  REGION 1..22: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRRRRRQRRRR

SEQ ID NO: 434 - protein, 20 aa, synthetic construct
  REGION 1..20: note = Description of Sequence: Synthetic peptide
  SITE 1: note = Nle
  SITE 3: note = D-leucine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXRRRRRQRRRR

SEQ ID NO: 435 - protein, 20 aa, synthetic construct
  REGION 1..20: note = Description of Sequence: Synthetic peptide
  SITE 1: note = Nle
  SITE 3: note = D-b-cyclohexylalanine
  SITE 5: note = D-phenylalanine
  SITE 9: note = bAla
XCXHXRWCXYGRRRRRQRRR

SEQ ID NO: 436 - protein, 18 aa, synthetic construct
  REGION 1..18: note = Description of Sequence: Synthetic peptide
  SITE 1: note = Nle
  SITE 3: note = D-b-cyclohexylalanine
  SITE 5: note = D-phenylalanine
  SITE 9: note = bAla
XCXHXRWCXRRRRRQRRR

SEQ ID NO: 437 - protein, 21 aa, synthetic construct
  REGION 1..21: note = Description of Sequence: Synthetic peptide
  SITE 1: note = Nle
  SITE 3: note = D-b-cyclohexylalanine
  SITE 5: note = D-phenylalanine
  SITE 9: note = bAla
  SITE 10: note = bAla
XCXHXRWCXXYGRRRRRQRRR

SEQ ID NO: 438 - protein, 19 aa, synthetic construct
  REGION 1..19: note = Description of Sequence: Synthetic peptide
  SITE 1: note = Nle
  SITE 3: note = D-b-cyclohexylalanine
  SITE 5: note = D-phenylalanine
  SITE 9: note = bAla
  SITE 10: note = bAla
XCXHXRWCXXRRRRRQRRR

SEQ ID NO: 439 - protein, 20 aa, synthetic construct
  REGION 1..20: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
XCXHXRWCXYGRRRRRQRRR

SEQ ID NO: 440 - protein, 18 aa, synthetic construct
  REGION 1..18: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
XCXHXRWCXRRRRRQRRR

SEQ ID NO: 441 - protein, 21 aa, synthetic construct
  REGION 1..21: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRRRRRQRRR

SEQ ID NO: 442 - protein, 19 aa, synthetic construct
  REGION 1..19: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXRRRRRQRRR

SEQ ID NO: 443 - protein, 21 aa, synthetic construct
  REGION 1..21: note = Description of Sequence: Synthetic peptide
  SITE 1: note = Nle
  SITE 3: note = D-b-cyclohexylalanine
  SITE 5: note = D-phenylalanine
  SITE 9: note = bAla
XCXHXRWCXYGRRRRRQRRRR

SEQ ID NO: 444 - protein, 19 aa, synthetic construct
  REGION 1..19: note = Description of Sequence: Synthetic peptide
  SITE 1: note = Nle
  SITE 3: note = D-b-cyclohexylalanine
  SITE 5: note = D-phenylalanine
  SITE 9: note = bAla
XCXHXRWCXRRRRRQRRRR

SEQ ID NO: 445 - protein, 22 aa, synthetic construct
  REGION 1..22: note = Description of Sequence: Synthetic peptide
  SITE 1: note = Nle
  SITE 3: note = D-b-cyclohexylalanine
  SITE 5: note = D-phenylalanine
  SITE 9: note = bAla
  SITE 10: note = bAla
XCXHXRWCXXYGRRRRRQRRRR

SEQ ID NO: 446 - protein, 20 aa, synthetic construct
  REGION 1..20: note = Description of Sequence: Synthetic peptide
  SITE 1: note = Nle
  SITE 3: note = D-b-cyclohexylalanine
  SITE 5: note = D-phenylalanine
  SITE 9: note = bAla
  SITE 10: note = bAla
XCXHXRWCXXRRRRRQRRRR

SEQ ID NO: 447 - protein, 21 aa, synthetic construct
  REGION 1..21: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
XCXHXRWCXYGRRRRRQRRRR

SEQ ID NO: 448 - protein, 19 aa, synthetic construct
  REGION 1..19: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
XCXHXRWCXRRRRRQRRRR

SEQ ID NO: 449 - protein, 22 aa, synthetic construct
  REGION 1..22: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXYGRRRRRQRRRR

SEQ ID NO: 450 - protein, 20 aa, synthetic construct
  REGION 1..20: note = Description of Sequence: Synthetic peptide
  SITE 1: note = Nle
  SITE 3: note = D-b-cyclohexylalanine
  SITE 5: note = D-phenylalanine
  SITE 9: note = 8-amino-3,6-dioxaoctanoic acid
  SITE 10: note = 8-amino-3,6-dioxaoctanoic acid
XCXHXRWCXXRRRRRQRRRR

SEQ ID NO: 451 - protein, 20 aa, synthetic construct
  REGION 1..20: note = Description of Sequence: Synthetic peptide
  SITE 1: note = Nle
  SITE 4: note = D-phenylalanine
  SITE 7: note = 4Abu
  SITE 9: note = bAla
XCHXRWXCXYGRRRRRQRRR

SEQ ID NO: 452 - protein, 18 aa, synthetic construct
  REGION 1..18: note = Description of Sequence: Synthetic peptide
  SITE 1: note = Nle
  SITE 4: note = D-phenylalanine
  SITE 7: note = 4Abu
  SITE 9: note = bAla
XCHXRWXCXRRRRRQRRR

SEQ ID NO: 453 - protein, 21 aa, synthetic construct
  REGION 1..21: note = Description of Sequence: Synthetic peptide
  SITE 1: note = Nle
  SITE 4: note = D-phenylalanine
  SITE 7: note = 4Abu
  SITE 9: note = bAla
  SITE 10: note = bAla
XCHXRWXCXXYGRRRRRQRRR

SEQ ID NO: 454 - protein, 19 aa, synthetic construct
  REGION 1..19: note = Description of Sequence: Synthetic peptide
  SITE 1: note = Nle
  SITE 4: note = D-phenylalanine
  SITE 7: note = 4Abu
  SITE 9: note = bAla
  SITE 10: note = bAla
XCHXRWXCXXRRRRRQRRR

SEQ ID NO: 455 - protein, 21 aa, synthetic construct
  REGION 1..21: note = Description of Sequence: Synthetic peptide
  SITE 1: note = Nle
  SITE 4: note = D-phenylalanine
  SITE 7: note = 4Abu
  SITE 9: note = bAla
XCHXRWXCXYGRRRRRQRRRR

SEQ ID NO: 456 - protein, 19 aa, synthetic construct
  REGION 1..19: note = Description of Sequence: Synthetic peptide
  SITE 1: note = Nle
  SITE 4: note = D-phenylalanine
  SITE 7: note = 4Abu
  SITE 9: note = bAla
XCHXRWXCXRRRRRQRRRR

SEQ ID NO: 457 - protein, 22 aa, synthetic construct
  REGION 1..22: note = Description of Sequence: Synthetic peptide
  SITE 1: note = Nle
  SITE 4: note = D-phenylalanine
  SITE 7: note = 4Abu
  SITE 9: note = bAla
  SITE 10: note = bAla
XCHXRWXCXXYGRRRRRQRRRR

SEQ ID NO: 458 - protein, 20 aa, synthetic construct
  REGION 1..20: note = Description of Sequence: Synthetic peptide
  SITE 1: note = Nle
  SITE 4: note = D-phenylalanine
  SITE 7: note = 4Abu
  SITE 9: note = bAla
  SITE 10: note = bAla
XCHXRWXCXXRRRRRQRRRR

SEQ ID NO: 459 - protein, 20 aa, synthetic construct
  REGION 1..20: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
XCHXRWXCXYGRRRRRQRRR

SEQ ID NO: 460 - protein, 18 aa, synthetic construct
  REGION 1..18: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
XCHXRWXCXRRRRRQRRR

SEQ ID NO: 461 - protein, 21 aa, synthetic construct
  REGION 1..21: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XCHXRWXCXXYGRRRRRQRRR

SEQ ID NO: 462 - protein, 19 aa, synthetic construct
  REGION 1..19: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XCHXRWXCXXRRRRRQRRR

SEQ ID NO: 463 - protein, 21 aa, synthetic construct
  REGION 1..21: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
XCHXRWXCXYGRRRRRQRRRR

SEQ ID NO: 464 - protein, 19 aa, synthetic construct
  REGION 1..19: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
XCHXRWXCXRRRRRQRRRR

SEQ ID NO: 465 - protein, 22 aa, synthetic construct
  REGION 1..22: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XCHXRWXCXXYGRRRRRQRRRR

SEQ ID NO: 466 - protein, 20 aa, synthetic construct
  REGION 1..20: note = Description of Sequence: Synthetic peptide
  SITE 1: note = Nle
  SITE 4: note = D-phenylalanine
  SITE 7: note = 4Abu
  SITE 9: note = 8-amino-3,6-dioxaoctanoic acid
  SITE 10: note = 8-amino-3,6-dioxaoctanoic acid
XCHXRWXCXXRRRRRQRRRR

SEQ ID NO: 467 - protein, 12 aa, synthetic construct
  REGION 1..12: note = Description of Sequence: Synthetic peptide
  SITE 4: note = D-4Br-Phe
CEHXRWGCPPKD

SEQ ID NO: 468 - protein, 12 aa, synthetic construct
  REGION 1..12: note = Description of Sequence: Synthetic peptide
  SITE 4: note = D-b-(2-naphthyl) alanine
CEHXRWACPPKD

SEQ ID NO: 469 - protein, 12 aa, synthetic construct
  REGION 1..12: note = Description of Sequence: Synthetic peptide
  SITE 4: note = D-b-(2-naphthyl) alanine
  SITE 6: note = b-(2-naphthyl) alanine
CEHXRXACPPKD

SEQ ID NO: 470 - protein, 12 aa, synthetic construct
  REGION 1..12: note = Description of Sequence: Synthetic peptide
  SITE 4: note = D-b-(2-naphthyl) alanine
  SITE 6: note = b-(1-naphthyl) alanine
CEHXRXACPPKD

SEQ ID NO: 471 - protein, 12 aa, synthetic construct
  REGION 1..12: note = Description of Sequence: Synthetic peptide
  SITE 4: note = D-b-(2-naphthyl) alanine
  SITE 6: note = 3-benzothienylalanine
CEHXRXACPPKD

SEQ ID NO: 472 - protein, 12 aa, synthetic construct
  REGION 1..12: note = Description of Sequence: Synthetic peptide
  SITE 4: note = D-b-(2-naphthyl) alanine
  SITE 6: note = b-(2-naphthyl) alanine
  SITE 7: note = bAla
CEHXRXXCPPKD

SEQ ID NO: 473 - protein, 12 aa, synthetic construct
  REGION 1..12: note = Description of Sequence: Synthetic peptide
  SITE 4: note = D-b-(2-naphthyl) alanine
  SITE 6: note = b-(2-naphthyl) alanine
  SITE 7: note = Aib
CEHXRXXCPPKD

SEQ ID NO: 474 - protein, 7 aa, synthetic construct
  REGION 1..7: note = Description of Sequence: Synthetic peptide
  SITE 2: note = D-Alanine
  SITE 4: note = D-phenylalanine
CXHXRWC

SEQ ID NO: 475 - protein, 7 aa, synthetic construct
  REGION 1..7: note = Description of Sequence: Synthetic peptide
  SITE 2: note = D-Alanine
  SITE 4: note = D-phenylalanine
CXHXRWC

SEQ ID NO: 476 - protein, 7 aa, synthetic construct
  REGION 1..7: note = Description of Sequence: Synthetic peptide
  SITE 2: note = D-Alanine
  SITE 4: note = D-b-(2-naphthyl) alanine
CXHXRWC

SEQ ID NO: 477 - protein, 7 aa, synthetic construct
  REGION 1..7: note = Description of Sequence: Synthetic peptide
  SITE 2: note = D-Alanine
  SITE 4: note = D-b-(2-naphthyl) alanine
CXHXRWC

SEQ ID NO: 478 - protein, 7 aa, synthetic construct
  REGION 1..7: note = Description of Sequence: Synthetic peptide
  SITE 2: note = D-Alanine
  SITE 4: note = D-phenylalanine
DXHXRWK

SEQ ID NO: 479 - protein, 7 aa, synthetic construct
  REGION 1..7: note = Description of Sequence: Synthetic peptide
  SITE 2: note = D-Alanine
  SITE 4: note = D-phenylalanine
  SITE 7: note = Orn
DXHXRWX

SEQ ID NO: 480 - protein, 7 aa, synthetic construct
  REGION 1..7: note = Description of Sequence: Synthetic peptide
  SITE 2: note = D-Alanine
  SITE 4: note = D-phenylalanine
  SITE 7: note = Dbu
DXHXRWX

SEQ ID NO: 481 - protein, 7 aa, synthetic construct
  REGION 1..7: note = Description of Sequence: Synthetic peptide
  SITE 2: note = D-Alanine
  SITE 4: note = D-phenylalanine
  MOD_RES 7: note = 2,3-diaminopropionic acid
DXHXRWX

SEQ ID NO: 482 - protein, 6 aa, synthetic construct
  REGION 1..6: note = Description of Sequence: Synthetic peptide
  SITE 3: note = D-b-(2-naphthyl) alanine
DHXRWK

SEQ ID NO: 483 - protein, 6 aa, synthetic construct
  REGION 1..6: note = Description of Sequence: Synthetic peptide
  SITE 3: note = D-phenylalanine
DHXRWK

SEQ ID NO: 484 - protein, 6 aa, synthetic construct
  REGION 1..6: note = Description of Sequence: Synthetic peptide
  MOD_RES 2: note = 1-amino-1-cyclopropanecarboxylic acid
  SITE 3: note = D-phenylalanine
DXFRWK

SEQ ID NO: 485 - protein, 6 aa, synthetic construct
  REGION 1..6: note = Description of Sequence: Synthetic peptide
  MOD_RES 2: note = 1-amino-1-cyclopentanecarboxylic acid
  SITE 3: note = D-phenylalanine
DXXRWK

SEQ ID NO: 486 - protein, 6 aa, synthetic construct
  REGION 1..6: note = Description of Sequence: Synthetic peptide
  SITE 2: note = 1-amino-1-cyclohexanecarboxylic acid
  SITE 3: note = D-phenylalanine
DXXRWK

SEQ ID NO: 487 - protein, 6 aa, synthetic construct
  REGION 1..6: note = Description of Sequence: Synthetic peptide
  SITE 2: note = 1-amino-1-cyclopropanecarboxylic acid
  SITE 3: note = D-b-(2-naphthyl) alanine
DXXRWK

SEQ ID NO: 488 - protein, 6 aa, synthetic construct
  REGION 1..6: note = Description of Sequence: Synthetic peptide
  MOD_RES 2: note = 1-amino-1-cyclopentanecarboxylic acid
  SITE 3: note = D-b-(2-naphthyl) alanine
DXXRWK

SEQ ID NO: 489 - protein, 6 aa, synthetic construct
  REGION 1..6: note = Description of Sequence: Synthetic peptide
  SITE 2: note = 1-amino-1-cyclohexanecarboxylic acid
  SITE 3: note = D-b-(2-naphthyl) alanine
DXXRWK

SEQ ID NO: 490 - protein, 6 aa, synthetic construct
  REGION 1..6: note = Description of Sequence: Synthetic peptide
  MOD_RES 2: note = 2-aminoindan-2-carboxylic acid
  SITE 3: note = D-phenylalanine
DXXRWK

SEQ ID NO: 491 - protein, 6 aa, synthetic construct
  REGION 1..6: note = Description of Sequence: Synthetic peptide
  SITE 2: note = 4-aminopiperidine-4-carboxylate
  SITE 3: note = D-phenylalanine
DXXRWK

SEQ ID NO: 492 - protein, 6 aa, synthetic construct
  REGION 1..6: note = Description of Sequence: Synthetic peptide
  MOD_RES 2: note = 2-aminoindan-2-carboxylic acid
  SITE 3: note = D-b-(2-naphthyl) alanine
DXXRWK

SEQ ID NO: 493 - protein, 6 aa, synthetic construct
  REGION 1..6: note = Description of Sequence: Synthetic peptide
  SITE 2: note = 4-aminopiperidine-4-carboxylate
  SITE 3: note = D-b-(2-naphthyl) alanine
DXXRWK

SEQ ID NO: 494 - protein, 7 aa, synthetic construct
  REGION 1..7: note = Description of Sequence: Synthetic peptide
  SITE 2: note = D-Alanine
  SITE 4: note = D-phenylalanine
  SITE 7: note = Orn
EXHXRWX

SEQ ID NO: 495 - protein, 7 aa, synthetic construct
  REGION 1..7: note = Description of Sequence: Synthetic peptide
  SITE 2: note = D-Alanine
  SITE 4: note = D-phenylalanine
  SITE 7: note = Dbu
EXHXRWX

SEQ ID NO: 496 - protein, 7 aa, synthetic construct
  REGION 1..7: note = Description of Sequence: Synthetic peptide
  SITE 2: note = D-Alanine
  SITE 4: note = D-phenylalanine
  MOD_RES 7: note = 2,3-diaminopropionic acid
EXHFRWX

SEQ ID NO: 497 - protein, 7 aa, synthetic construct
  REGION 1..7: note = Description of Sequence: Synthetic peptide
  SITE 2: note = D-Alanine
  SITE 4: note = D-phenylalanine
EXHXRWK

SEQ ID NO: 498 - protein, 6 aa, synthetic construct
  REGION 1..6: note = Description of Sequence: Synthetic peptide
  SITE 3: note = D-phenylalanine
  MOD_RES 6: note = 2,3-diaminopropionic acid
EHFRWX

SEQ ID NO: 499 - protein, 6 aa, synthetic construct
  REGION 1..6: note = Description of Sequence: Synthetic peptide
  SITE 3: note = D-phenylalanine
EHXRWK

SEQ ID NO: 500 - protein, 9 aa, synthetic construct
  REGION 1..9: note = Description of Sequence: Synthetic peptide
  SITE 6: note = D-phenylalanine
RGCEHXRWC

SEQ ID NO: 501 - protein, 9 aa, synthetic construct
  REGION 1..9: note = Description of Sequence: Synthetic peptide
  SITE 1: note = Nle
  SITE 6: note = D-phenylalanine
XGCEHXRWC

SEQ ID NO: 502 - protein, 9 aa, synthetic construct
  REGION 1..9: note = Description of Sequence: Synthetic peptide
  SITE 6: note = D-phenylalanine
GGCEHXRWC

SEQ ID NO: 503 - protein, 9 aa, synthetic construct
  REGION 1..9: note = Description of Sequence: Synthetic peptide
  SITE 1: note = Nle
  SITE 4: note = D-Alanine
  SITE 6: note = D-phenylalanine
XGCXHXRWC

SEQ ID NO: 504 - protein, 9 aa, synthetic construct
  REGION 1..9: note = Description of Sequence: Synthetic peptide
  SITE 4: note = D-Alanine
  SITE 6: note = D-phenylalanine
GGCXHXRWC

SEQ ID NO: 505 - protein, 9 aa, synthetic construct
  REGION 1..9: note = Description of Sequence: Synthetic peptide
  SITE 1: note = Nle
  SITE 4: note = D-Alanine
  SITE 6: note = D-phenylalanine
  SITE 9: note = Penicillamine
XGCXHXRWX

SEQ ID NO: 506 - protein, 9 aa, synthetic construct
  REGION 1..9: note = Description of Sequence: Synthetic peptide
  SITE 4: note = D-Alanine
  SITE 6: note = D-phenylalanine
  SITE 9: note = Penicillamine
GGCXHXRWX

SEQ ID NO: 507 - protein, 9 aa, synthetic construct
  REGION 1..9: note = Description of Sequence: Synthetic peptide
  SITE 4: note = D-Alanine
  SITE 6: note = D-phenylalanine
AGCXHXRWC

SEQ ID NO: 508 - protein, 9 aa, synthetic construct
  REGION 1..9: note = Description of Sequence: Synthetic peptide
  SITE 1: note = D-Alanine
  SITE 4: note = D-Alanine
  SITE 6: note = D-phenylalanine
XGCXHXRWC

SEQ ID NO: 509 - protein, 9 aa, synthetic construct
  REGION 1..9: note = Description of Sequence: Synthetic peptide
  SITE 1: note = Aib
  SITE 4: note = D-Alanine
  SITE 6: note = D-phenylalanine
XGCXHXRWC

SEQ ID NO: 510 - protein, 9 aa, synthetic construct
  REGION 1..9: note = Description of Sequence: Synthetic peptide
  SITE 4: note = D-Alanine
  SITE 6: note = D-phenylalanine
VGCXHXRWC

SEQ ID NO: 511 - protein, 9 aa, synthetic construct
  REGION 1..9: note = Description of Sequence: Synthetic peptide
  SITE 4: note = D-Alanine
  SITE 6: note = D-phenylalanine
IGCXHXRWC

SEQ ID NO: 512 - protein, 9 aa, synthetic construct
  REGION 1..9: note = Description of Sequence: Synthetic peptide
  SITE 4: note = D-Alanine
  SITE 6: note = D-phenylalanine
LGCXHXRWC

SEQ ID NO: 513 - protein, 9 aa, synthetic construct
  REGION 1..9: note = Description of Sequence: Synthetic peptide
  SITE 6: note = D-b-(2-naphthyl) alanine
GGCEHXRWC

SEQ ID NO: 514 - protein, 9 aa, synthetic construct
  REGION 1..9: note = Description of Sequence: Synthetic peptide
  SITE 1: note = Nle
  SITE 6: note = D-b-(2-naphthyl) alanine
XGCEHXRWC

SEQ ID NO: 515 - protein, 9 aa, synthetic construct
  REGION 1..9: note = Description of Sequence: Synthetic peptide
  SITE 1: note = D-arginine
  SITE 6: note = D-phenylalanine
XGCEHXRWC

SEQ ID NO: 516 - protein, 9 aa, synthetic construct
  REGION 1..9: note = Description of Sequence: Synthetic peptide
  SITE 1: note = D-arginine
  SITE 4: note = D-Alanine
  SITE 6: note = D-phenylalanine
XGCXHXRWC

SEQ ID NO: 517 - protein, 9 aa, synthetic construct
  REGION 1..9: note = Description of Sequence: Synthetic peptide
  SITE 4: note = D-Alanine
  SITE 6: note = D-phenylalanine
RGCXHXRWC

SEQ ID NO: 518 - protein, 9 aa, synthetic construct
  REGION 1..9: note = Description of Sequence: Synthetic peptide
  SITE 1: note = D-arginine
  SITE 4: note = D-Alanine
  SITE 6: note = D-b-(2-naphthyl) alanine
XGCXHXRWC

SEQ ID NO: 519 - protein, 9 aa, synthetic construct
  REGION 1..9: note = Description of Sequence: Synthetic peptide
  SITE 4: note = D-Alanine
  SITE 6: note = D-b-(2-naphthyl) alanine
RGCXHXRWC

SEQ ID NO: 520 - protein, 9 aa, synthetic construct
  REGION 1..9: note = Description of Sequence: Synthetic peptide
  SITE 2: note = Nle
  SITE 6: note = D-phenylalanine
AXCEHXRWC

SEQ ID NO: 521 - protein, 9 aa, synthetic construct
  REGION 1..9: note = Description of Sequence: Synthetic peptide
  SITE 2: note = Nle
  SITE 6: note = D-phenylalanine
VXCEHXRWC

SEQ ID NO: 522 - protein, 9 aa, synthetic construct
  REGION 1..9: note = Description of Sequence: Synthetic peptide
  SITE 2: note = Nle
  SITE 6: note = D-phenylalanine
GXCEHXRWC

SEQ ID NO: 523 - protein, 9 aa, synthetic construct
  REGION 1..9: note = Description of Sequence: Synthetic peptide
  SITE 1: note = 1-amino-1-cyclohexanecarboxylic acid
  SITE 2: note = Nle
  SITE 4: note = D-Alanine
  SITE 6: note = D-phenylalanine
XXCXHXRWC

SEQ ID NO: 524 - protein, 9 aa, synthetic construct
  REGION 1..9: note = Description of Sequence: Synthetic peptide
  SITE 2: note = Nle
  SITE 4: note = D-Alanine
  SITE 6: note = D-phenylalanine
GXCXHXRWC

SEQ ID NO: 525 - protein, 9 aa, synthetic construct
  REGION 1..9: note = Description of Sequence: Synthetic peptide
  SITE 2: note = Nle
  SITE 4: note = D-Alanine
  SITE 6: note = D-phenylalanine
AXCXHXRWC

SEQ ID NO: 526 - protein, 9 aa, synthetic construct
  REGION 1..9: note = Description of Sequence: Synthetic peptide
  SITE 1: note = D-Alanine
  SITE 2: note = Nle
  SITE 4: note = D-Alanine
  SITE 6: note = D-phenylalanine
XXCXHXRWC

SEQ ID NO: 527 - protein, 9 aa, synthetic construct
  REGION 1..9: note = Description of Sequence: Synthetic peptide
  SITE 2: note = Nle
  SITE 4: note = D-Alanine
  SITE 6: note = D-phenylalanine
VXCXHXRWC

SEQ ID NO: 528 - protein, 9 aa, synthetic construct
  REGION 1..9: note = Description of Sequence: Synthetic peptide
  SITE 2: note = Nle
  SITE 4: note = D-Alanine
  SITE 6: note = D-phenylalanine
LXCXHXRWC

SEQ ID NO: 529 - protein, 9 aa, synthetic construct
  REGION 1..9: note = Description of Sequence: Synthetic peptide
  SITE 1: note = b-cyclohexylalanine
  SITE 2: note = Nle
  SITE 4: note = D-Alanine
  SITE 6: note = D-phenylalanine
XXCXHXRWC

SEQ ID NO: 530 - protein, 9 aa, synthetic construct
  REGION 1..9: note = Description of Sequence: Synthetic peptide
  SITE 1: note = Aib
  SITE 2: note = Nle
  SITE 4: note = D-Alanine
  SITE 6: note = D-phenylalanine
XXCXHXRWC

SEQ ID NO: 531 - protein, 9 aa, synthetic construct
  REGION 1..9: note = Description of Sequence: Synthetic peptide
  SITE 6: note = D-phenylalanine
GRCEHXRWC

SEQ ID NO: 532 - protein, 9 aa, synthetic construct
  REGION 1..9: note = Description of Sequence: Synthetic peptide
  SITE 6: note = D-b-(2-naphthyl) alanine
GRCEHXRWC

SEQ ID NO: 533 - protein, 9 aa, synthetic construct
  REGION 1..9: note = Description of Sequence: Synthetic peptide
  SITE 4: note = D-Alanine
  SITE 6: note = D-phenylalanine
GRCXHXRWC

SEQ ID NO: 534 - protein, 9 aa, synthetic construct
  REGION 1..9: note = Description of Sequence: Synthetic peptide
  SITE 4: note = D-Alanine
  SITE 6: note = D-b-(2-naphthyl) alanine
GRCXHXRWC

SEQ ID NO: 535 - protein, 9 aa, synthetic construct
  REGION 1..9: note = Description of Sequence: Synthetic peptide
  SITE 2: note = D-arginine
  SITE 6: note = D-phenylalanine
GXCEHXRWC

SEQ ID NO: 536 - protein, 9 aa, synthetic construct
  REGION 1..9: note = Description of Sequence: Synthetic peptide
  SITE 2: note = D-arginine
  SITE 4: note = D-Alanine
  SITE 6: note = D-phenylalanine
GXCXHXRWC

SEQ ID NO: 537 - protein, 9 aa, synthetic construct
  REGION 1..9: note = Description of Sequence: Synthetic peptide
  SITE 2: note = D-arginine
  SITE 4: note = D-Alanine
  SITE 6: note = D-b-(2-naphthyl) alanine
GXCXHXRWC

SEQ ID NO: 538 - protein, 9 aa, synthetic construct
  REGION 1..9: note = Description of Sequence: Synthetic peptide
  SITE 1: note = Nle
  SITE 6: note = D-phenylalanine
XACEHXRWC

SEQ ID NO: 539 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
XCXHXRWC

SEQ ID NO: 540 - protein, 8 aa, synthetic construct
  REGION 1..8: note = Description of Sequence: Synthetic peptide
  SITE 3: note = D-Alanine
  SITE 5: note = D-phenylalanine
ACXHXRWC

SEQ ID NO: 541 - protein, 8 aa, synthetic construct
  REGION 1..8: note = Description of Sequence: Synthetic peptide
  SITE 1: note = D-Alanine
  SITE 3: note = D-Alanine
  SITE 5: note = D-phenylalanine
XCXHXRWC

SEQ ID NO: 542 - protein, 8 aa, synthetic construct
  REGION 1..8: note = Description of Sequence: Synthetic peptide
  SITE 1: note = Aib
  SITE 3: note = D-Alanine
  SITE 5: note = D-phenylalanine
XCXHXRWC

SEQ ID NO: 543 - protein, 8 aa, synthetic construct
  REGION 1..8: note = Description of Sequence: Synthetic peptide
  SITE 3: note = D-Alanine
  SITE 5: note = D-phenylalanine
VCXHXRWC

SEQ ID NO: 544 - protein, 8 aa, synthetic construct
  REGION 1..8: note = Description of Sequence: Synthetic peptide
  SITE 1: note = Abu
  SITE 3: note = D-Alanine
  SITE 5: note = D-phenylalanine
XCXHXRWC

SEQ ID NO: 545 - protein, 8 aa, synthetic construct
  REGION 1..8: note = Description of Sequence: Synthetic peptide
  SITE 3: note = D-Alanine
  SITE 5: note = D-phenylalanine
LCXHXRWC

SEQ ID NO: 546 - protein, 8 aa, synthetic construct
  REGION 1..8: note = Description of Sequence: Synthetic peptide
  SITE 3: note = D-Alanine
  SITE 5: note = D-phenylalanine
ICXHXRWC

SEQ ID NO: 547 - protein, 8 aa, synthetic construct
  REGION 1..8: note = Description of Sequence: Synthetic peptide
  SITE 1: note = b-cyclohexylalanine
  SITE 3: note = D-Alanine
  SITE 5: note = D-phenylalanine
XCXHXRWC

SEQ ID NO: 548 - protein, 8 aa, synthetic construct
  REGION 1..8: note = Description of Sequence: Synthetic peptide
  SITE 1: note = 1-amino-1-cyclohexanecarboxylic acid
  SITE 3: note = D-Alanine
  SITE 5: note = D-phenylalanine
XCXHXRWC

SEQ ID NO: 549 - protein, 8 aa, synthetic construct
  REGION 1..8: note = Description of Sequence: Synthetic peptide
  SITE 3: note = D-Alanine
  SITE 5: note = D-phenylalanine
FCXHXRWC

SEQ ID NO: 550 - protein, 8 aa, synthetic construct
  REGION 1..8: note = Description of Sequence: Synthetic peptide
  SITE 3: note = D-Alanine
  SITE 5: note = D-phenylalanine
GCXHXRWC

SEQ ID NO: 551 - protein, 8 aa, synthetic construct
  REGION 1..8: note = Description of Sequence: Synthetic peptide
  SITE 5: note = D-phenylalanine
GCEHXRWC

SEQ ID NO: 552 - protein, 9 aa, synthetic construct
  REGION 1..9: note = Description of Sequence: Synthetic peptide
  SITE 4: note = D-Alanine
  SITE 6: note = D-phenylalanine
YRCXHXRWC

SEQ ID NO: 553 - protein, 9 aa, synthetic construct
  REGION 1..9: note = Description of Sequence: Synthetic peptide
  SITE 1: note = b-(2-naphthyl) alanine
  SITE 4: note = D-Alanine
  SITE 6: note = D-phenylalanine
XRCXHXRWC

SEQ ID NO: 554 - protein, 9 aa, synthetic construct
  REGION 1..9: note = Description of Sequence: Synthetic peptide
  SITE 1: note = b-(1-naphthyl) alanine
  SITE 4: note = D-Alanine
  SITE 6: note = D-phenylalanine
XRCXHXRWC

SEQ ID NO: 555 - protein, 9 aa, synthetic construct
  REGION 1..9: note = Description of Sequence: Synthetic peptide
  SITE 4: note = D-Alanine
  SITE 6: note = D-phenylalanine
FRCXHXRWC

SEQ ID NO: 556 - protein, 9 aa, synthetic construct
  REGION 1..9: note = Description of Sequence: Synthetic peptide
  SITE 4: note = D-Alanine
  SITE 6: note = D-phenylalanine
WRCXHXRWC

SEQ ID NO: 557 - protein, 9 aa, synthetic construct
  REGION 1..9: note = Description of Sequence: Synthetic peptide
  SITE 1: note = (S)-pentafluorophenylalanine
  SITE 4: note = D-Alanine
  SITE 6: note = D-phenylalanine
ARCXHXRWC

SEQ ID NO: 558 - protein, 9 aa, synthetic construct
  REGION 1..9: note = Description of Sequence: Synthetic peptide
  SITE 4: note = D-Alanine
  SITE 6: note = D-phenylalanine
HRCXHXRWC

SEQ ID NO: 559 - protein, 9 aa, synthetic construct
  REGION 1..9: note = Description of Sequence: Synthetic peptide
  SITE 4: note = D-Alanine
  SITE 6: note = D-phenylalanine
HRCXHXRWC

SEQ ID NO: 560 - protein, 9 aa, synthetic construct
  REGION 1..9: note = Description of Sequence: Synthetic peptide
RKKRRQRRR

SEQ ID NO: 561 - protein, 11 aa, synthetic construct
  REGION 1..11: note = Description of Sequence: Synthetic peptide
YARKARRQARR

SEQ ID NO: 562 - protein, 11 aa, synthetic construct
  REGION 1..11: note = Description of Sequence: Synthetic peptide
YARAARRAARR

SEQ ID NO: 563 - protein, 11 aa, synthetic construct
  REGION 1..11: note = Description of Sequence: Synthetic peptide
YARRRRRRRRR

SEQ ID NO: 564 - protein, 11 aa, synthetic construct
  REGION 1..11: note = Description of Sequence: Synthetic peptide
YAAARRRRRRR

SEQ ID NO: 565 - protein, 11 aa, synthetic construct
  REGION 1..11: note = Description of Sequence: Synthetic peptide
YARAPRRARRR

SEQ ID NO: 566 - protein, 10 aa, synthetic construct
  REGION 1..10: note = Description of Sequence: Synthetic peptide
YARAPRRPRR

SEQ ID NO: 567 - protein, 9 aa, synthetic construct
  REGION 1..9: note = Description of Sequence: Synthetic peptide
RKQKRRRRR

SEQ ID NO: 568 - protein, 9 aa, synthetic construct
  REGION 1..9: note = Description of Sequence: Synthetic peptide
RKKRQRRRR

SEQ ID NO: 569 - protein, 9 aa, synthetic construct
  REGION 1..9: note = Description of Sequence: Synthetic peptide
RKKRRRQRR

SEQ ID NO: 570 - protein, 9 aa, synthetic construct
  REGION 1..9: note = Description of Sequence: Synthetic peptide
RKKRRRRQR

SEQ ID NO: 571 - protein, 9 aa, synthetic construct
  REGION 1..9: note = Description of Sequence: Synthetic peptide
RKKRRRRRQ

SEQ ID NO: 572 - protein, 9 aa, synthetic construct
  REGION 1..9: note = Description of Sequence: Synthetic peptide
RKKQRRRRR

SEQ ID NO: 573 - protein, 9 aa, synthetic construct
  REGION 1..9: note = Description of Sequence: Synthetic peptide
RQKKRRRRR

SEQ ID NO: 574 - protein, 9 aa, synthetic construct
  REGION 1..9: note = Description of Sequence: Synthetic peptide
RQRRRRRRR

SEQ ID NO: 575 - protein, 10 aa, synthetic construct
  REGION 1..10: note = Description of Sequence: Synthetic peptide
RQRRRRRRRR

SEQ ID NO: 576 - protein, 9 aa, synthetic construct
  REGION 1..9: note = Description of Sequence: Synthetic peptide
RRQRRRRRR

SEQ ID NO: 577 - protein, 10 aa, synthetic construct
  REGION 1..10: note = Description of Sequence: Synthetic peptide
RRQRRRRRRR

SEQ ID NO: 578 - protein, 9 aa, synthetic construct
  REGION 1..9: note = Description of Sequence: Synthetic peptide
RRRQRRRRR

SEQ ID NO: 579 - protein, 10 aa, synthetic construct
  REGION 1..10: note = Description of Sequence: Synthetic peptide
RRRQRRRRRR

SEQ ID NO: 580 - protein, 9 aa, synthetic construct
  REGION 1..9: note = Description of Sequence: Synthetic peptide
RRRRQRRRR

SEQ ID NO: 581 - protein, 10 aa, synthetic construct
  REGION 1..10: note = Description of Sequence: Synthetic peptide
RRRRQRRRRR

SEQ ID NO: 582 - protein, 5 aa, synthetic construct
  REGION 1..5: note = Description of Sequence: Synthetic peptide
RRRRR

SEQ ID NO: 583 - protein, 9 aa, synthetic construct
  REGION 1..9: note = Description of Sequence: Synthetic peptide
RRRRRQRRR

SEQ ID NO: 584 - protein, 10 aa, synthetic construct
  REGION 1..10: note = Description of Sequence: Synthetic peptide
RRRRRQRRRR

SEQ ID NO: 585 - protein, 6 aa, synthetic construct
  REGION 1..6: note = Description of Sequence: Synthetic peptide
RRRRRR

SEQ ID NO: 586 - protein, 10 aa, synthetic construct
  REGION 1..10: note = Description of Sequence: Synthetic peptide
RRRRRRQRRR

SEQ ID NO: 587 - protein, 7 aa, synthetic construct
  REGION 1..7: note = Description of Sequence: Synthetic peptide
RRRRRRR

SEQ ID NO: 588 - protein, 10 aa, synthetic construct
  REGION 1..10: note = Description of Sequence: Synthetic peptide
RRRRRRRQRR

SEQ ID NO: 589 - protein, 8 aa, synthetic construct
  REGION 1..8: note = Description of Sequence: Synthetic peptide
RRRRRRRR

SEQ ID NO: 590 - protein, 9 aa, synthetic construct
  REGION 1..9: note = Description of Sequence: Synthetic peptide
  VARIANT 1: note = This position me be absent
  VARIANT 2: note = This position me be absent
  VARIANT 3: note = This position me be absent
  VARIANT 4: note = This position me be absent
  VARIANT 5: note = This position me be absent
  VARIANT 6: note = This position me be absent
  VARIANT 7: note = This position me be absent
RRRRRRRQR

SEQ ID NO: 591 - protein, 9 aa, synthetic construct
  REGION 1..9: note = Description of Sequence: Synthetic peptide
RRRRRRRRR

SEQ ID NO: 592 - protein, 10 aa, synthetic construct
  REGION 1..10: note = Description of Sequence: Synthetic peptide
RRRRRRRRRQ

SEQ ID NO: 593 - protein, 9 aa, synthetic construct
  REGION 1..9: note = Description of Sequence: Synthetic peptide
QRKKRRRRR

SEQ ID NO: 594 - protein, 9 aa, synthetic construct
  REGION 1..9: note = Description of Sequence: Synthetic peptide
QRRRRRRRR

SEQ ID NO: 595 - protein, 10 aa, synthetic construct
  REGION 1..10: note = Description of Sequence: Synthetic peptide
QRRRRRRRRR

SEQ ID NO: 596 - protein, 11 aa, synthetic construct
  REGION 1..11: note = Description of Sequence: Synthetic peptide
YGRKKRRQRRR

SEQ ID NO: 597 - protein, 12 aa, synthetic construct
  REGION 1..12: note = Description of Sequence: Synthetic peptide
  SITE 12: note = 8-amino-3,6-dioxaoctanoic acid
YGRKKRRQRRRX

SEQ ID NO: 598 - protein, 4 aa, synthetic construct
  REGION 1..4: note = Description of Sequence: Synthetic peptide
  SITE 1: note = bAla
  SITE 2: note = bAla
AAYG

SEQ ID NO: 599 - skipped

SEQ ID NO: 600 - protein, 9 aa, synthetic construct
  REGION 1..9: note = Description of Sequence: Synthetic peptide
RKRRRQRRR

SEQ ID NO: 601 - protein, 9 aa, synthetic construct
  REGION 1..9: note = Description of Sequence: Synthetic peptide
  VARIANT 3: note = This position may be absent
  VARIANT 4: note = This position may be absent
  VARIANT 5: note = This position may be absent
  VARIANT 6: note = This position may be absent
  VARIANT 7: note = This position may be absent
  VARIANT 8: note = This position may be absent
  VARIANT 9: note = This position may be absent
RQRRRRRRR

SEQ ID NO: 602 - protein, 9 aa, synthetic construct
  REGION 1..9: note = Description of Sequence: Synthetic peptide
RRKRRQRRR

SEQ ID NO: 603 - protein, 11 aa, synthetic construct
  REGION 1..11: note = Description of Sequence: Synthetic peptide
  VARIANT 1: note = Position may be absent
  VARIANT 2: note = Position may be absent
  VARIANT 3: note = Position may be absent
  VARIANT 4: note = Position may be absent
  VARIANT 5: note = Position may be absent
  VARIANT 6: note = Position may be absent
  VARIANT 7: note = Position may be absent
RRRRRRRQRRR

SEQ ID NO: 604 - protein, 9 aa, synthetic construct
  REGION 1..9: note = Description of Sequence: Synthetic peptide
  VARIANT 1: note = Position may be absent
  VARIANT 2: note = Position may be absent
  VARIANT 3: note = Position may be absent
  VARIANT 4: note = Position may be absent
  VARIANT 5: note = Position may be absent
  VARIANT 6: note = Position may be absent
  VARIANT 7: note = Position may be absent
RRRRRRRQR

SEQ ID NO: 605 - protein, 12 aa, synthetic construct
  REGION 1..12: note = Description of Sequence: Synthetic peptide
  SITE 4: note = D-isomer of halogen modified Phenylalanine
  VARIANT 4: note = Position may also be b-(2-naphthyl)alanine
  VARIANT 6: note = Position may also be 3- benzothienylalanine or b-(1-naphthyl)alanine or b-(2-naphthyl)alanine
  VARIANT 7: note = Position may be Aib or bAla or glycine
  REGION 1..12: note = Variant residues given in the sequence have no preference with respect to those in the annotations for variant positions"
CEHFRWACPPKD

SEQ ID NO: 606 - DNA, 21 bp, synthetic construct
  misc_feature 1..21: note = source = /note="Description of Artificial Sequence: Synthetic primer"
  source 1..21: mol_type = other DNA
tgctggattgcagagcagtaa

SEQ ID NO: 607 - DNA, 21 bp, synthetic construct
  misc_feature 1..21: note = source = /note="Description of Artificial Sequence: Synthetic primer"
  source 1..21: mol_type = other DNA
gcatgcagagattccgagaga

SEQ ID NO: 608 - protein, 5 aa, synthetic construct
  REGION 1..5: note = Description of Sequence: Synthetic peptide
  SITE 1..5: note = D-arginineinine
RRRRR

SEQ ID NO: 609 - protein, 6 aa, synthetic construct
  REGION 1..6: note = Description of Sequence: Synthetic peptide
  SITE 1..6: note = D-arginineinine
RRRRRR

SEQ ID NO: 610 - protein, 7 aa, synthetic construct
  REGION 1..7: note = Description of Sequence: Synthetic peptide
  SITE 1..7: note = D-arginineinine
RRRRRRR

SEQ ID NO: 611 - protein, 8 aa, synthetic construct
  REGION 1..8: note = Description of Sequence: Synthetic peptide
  SITE 1..8: note = D-arginineinine
RRRRRRRR

SEQ ID NO: 612 - protein, 9 aa, synthetic construct
  REGION 1..9: note = Description of Sequence: Synthetic peptide
  SITE 1..9: note = D-arginineinine
RRRRRRRRR

SEQ ID NO: 613 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
XCXHXRWC

SEQ ID NO: 614 - protein, 8 aa, synthetic construct
  REGION 1..8: note = Description of Sequence: Synthetic peptide
  SITE 1: note = Nle
  SITE 3: note = D-Alanine
  SITE 5: note = D-phenylalanine
XCXHXRWC

(19) United States
(12) Patent Application Publication
(10) Pub. No.: US 2025/0082717 A1
(43) Pub. Date: Mar. 13, 2025

(54) COMPOSITIONS FOR TREATING KIDNEY DISEASE

(71) Applicants: RHYTHM PHARMACEUTICALS, INC., Boston, MA (US); Institute National De La Santé Et De La Recherche Mêdicale, Paris (FR); UNIVERSITÉ DE STRASBOURG, Strasbourg (FR)

(72) Inventors: Leonardus H.T. Van Der Ploeg, Newton, MA (US); Alastair Garfield, Allston, MA (US); Vincent Marion, Strasbourg (FR)

(21) Appl. No.: 18/654,757

(22) Filed: May 3, 2024

Related U.S. Application Data
(63) Continuation of application No. 17/045,724, filed on Oct. 6, 2020, now abandoned.
(60) Provisional application No. 62/653,997, filed on Apr. 6, 2018.

(51) Int. Cl. A61K 38/12; A61P 3/04; A61P 13/12

(52) U.S. Cl. CPC A61K 38/12; A61P 3/04; A61P 13/12

(57) ABSTRACT

The disclosure is related to a method of treating chronic kidney disease in a subject with a melanocortin-4 receptor (MC4R) agonist, e.g., a compound of any one of Formulas (I), (II), (III), (IV), (V), (VI), (VII), (VIII), (X), or (XI), or a pharmaceutically acceptable salt thereof (e.g., as described herein).

### CLAIM OF PRIORITY

This application is a Continuation of U.S. application Ser. No. 17/045,724 filed Oct. 6, 2020, which is a U.S. National Phase Application under 35 U.S.C. § 371 of International Application No. PCT/US2019/026102, filed Apr. 5, 2019, which claims priority to U.S. Provisional Application No. 62/653,997, filed Apr. 6, 2018, which are incorporated herein by reference in their entireties.

### SEQUENCE LISTING

The instant application contains a Sequence Listing which has been submitted electronically in ASCII format and is hereby incorporated by reference in its entirety. Said ASCII copy, created on May 10, 2019, is named R2054-7021WO_SL.txt and is 292,780 bytes in size.

### BACKGROUND

Chronic kidney disease is a condition characterized by the slow loss of kidney function over time. It currently affects over 30 million American adults. Although the population of patients afflicted with CKD grows each year, there is no cure. Current treatments for CKD seek to manage co-morbidities and, if possible, slow the progression of the disease. However, as the disease progresses, renal function decreases and eventually renal replacement therapy is employed to compensate for lost kidney function. As such, there is a need for new therapies to treat chronic kidney disease and/or its related co-morbidities.

### SUMMARY

The present disclosure features methods for treating chronic kidney disease in a subject. In some embodiments, the method comprises administering a melanocortin 4 receptor (MC4R) agonist to the subject. In some embodiments, the MC4R agonist is a compound of any one of Formulas (I), (II), (III), (IV), (V), (VI), (VII), (VIII), (X), or (XI), (e.g., as described herein) or a pharmaceutically acceptable salt thereof. The method may further comprise acquiring a level of a biomarker (e.g., leptin, creatine, adiponectin, or a cytokine), and comparing the acquired level of a biomarker to a reference value.

In some embodiments, the subject may have renal dysfunction, cognitive impairment, or retinal degeneration. In some embodiments, the subject is obese. In some embodiments, the subject has Bardet-Biedl syndrome (BBS), Alstrom syndrome (ALMS), or another ciliopathy (e.g., a polycystic kidney disease (e.g., dominant (ADPKD for autosomal dominant polycystic kidney disease) or recessive (ARPKD for autosomal recessive polycystic kidney disease)), Joubert syndrome, Meckel-Gruber syndrome, or orofaciodigital syndrome 1).

In some embodiments, the subject has a body mass index (BMI) greater than 25 kg/m² (e.g., ≥25, 30, 31, 32, 33, 34, 35, 36, 37, 38, 39, 40, 41, 42, 43, 44, 45, 46, 47, 48, 49, 50 kg/m² or greater) prior to administration of the compound of any one of Formulas (I), (II), (III), (IV), (V), (VI), (VII), (VIII), (X), or (XI). In some embodiments, the subject has a body mass index (BMI) greater than 35 kg/m² (e.g., ≥36, 37, 38, 39, 40, 41, 42, 43, 44, 45, 46, 47, 48, 49, 50 kg/m² or greater) prior to administration of the compound of any one of Formulas (I), (II), (III), (IV), (V), (VI), (VII), (VIII), (X), or (XI). In some embodiments, the subject has a body mass index (BMI) greater than 45 kg/m² (e.g., ≥41, 42, 43, 44, 45, 46, 47, 48, 49, 50, 51, 52, 53, 54, 55 kg/m² or greater) prior to administration of the compound of any one of Formulas (I), (II), (III), (IV), (V), (VI), (VII), (VIII), (X), or (XI).

In some embodiments, the subject has failed one or more previous therapies, e.g., exercise, diet, or behavioral therapies, prior to administration of the compound of any one of Formulas (I), (II), (III), (IV), (V), (VI), (VII), (VIII), (X), or (XI), e.g., at the time the compound of any one of Formulas (I), (II), (III), (IV), (V), (VI), (VII), (VIII), (X), or (XI) is prescribed, or at the time of the first administration of the compound of any one of Formulas (I), (II), (III), (IV), (V), (VI), (VII), (VIII), (X), or (XI).

In some embodiments, prior to administration of a compound of any one of Formulas (I), (II), (III), (IV), (V), (VI), (VII), (VIII), (X), or (XI), the subject has an increased level of a biomarker relative to a reference level. In some embodiments, after administration of a compound of any one of Formulas (I), (II), (III), (IV), (V), (VI), (VII), (VIII), (X), or (XI), the subject has a decreased level of a biomarker relative to a reference level.

In some embodiments, the method further comprises acquiring the level of a biomarker. In some embodiments, the method further comprises comparing the acquired level to a reference value. In some embodiments, responsive to the comparison, a compound of any one of Formulas (I), (II), (III), (IV), (V), (VI), (VII), (VIII), (X), or (XI) is administered. In some embodiments, a dosage or treatment comprising a compound of any one of Formulas (I), (II), (III), (IV), (V), (VI), (VII), (VIII), (X), or (XI) is administered responsive to the level of a biomarker. In some embodiments, the amount of a dosage or treatment comprising a compound of any one of Formulas (I), (II), (III), (IV), (V), (VI), (VII), (VIII), (X), or (XI) is sufficient to decrease the level of a biomarker relative to a reference value.

In some embodiments, the biomarker is leptin. In some embodiments, the biomarker is creatine. In some embodiments, the biomarker is adiponectin. In some embodiments, the biomarker is glucose. In some embodiments, the biomarker is a salt, such as sodium, potassium, chloride, calcium, or phosphorus. In some embodiments, the biomarker is an inflammatory cytokine. In some embodiments, the inflammatory cytokine is a pro-inflammatory cytokine. In some embodiments, the pro-inflammatory cytokine comprises IL-6, MCP-1, or IL-23. In some embodiments, the biomarker is the level of protein in the urine. In some embodiments, the biomarker is the glomerular filtration rate (GFR). In some embodiments, the biomarker is a structural abnormality. In some embodiments, the subject has a structural abnormality in the kidney. In some embodiments, the structural abnormality comprises a fetal lobulation, a parenchymal cyst, a calyceal cyst, calyceal clubbing, or renal agenesia. In some embodiments, the subject is a mammal, e.g., a human.

In some embodiments, the compound is a compound of Formula (I) or a pharmaceutically acceptable salt thereof. In some embodiments, the compound of Formula (I) is Ac-Arg-c (Cys-D-Ala-His-D-Phe-Arg-Trp-Cys)-NH2 (SEQ ID NO: 140). In some embodiments, the compound is a compound of Formula (II) or a pharmaceutically acceptable salt thereof. In some embodiments, the compound of Formula (II) is Hydantoin (C(O)-(Arg-Gly))-cyclo(Cys-Glu-His-D-Phe-Arg-Trp-Cys)-NH₂ (SEQ ID NO:13). In some embodiments, the compound of any one of Formulas (I), (II), (III), (IV), (V), (VI), (VII), (VIII), (X), or (XI) is formulated as a pharmaceutical composition.

In another aspect, the present disclosure features a method of evaluating a subject for treatment of chronic kidney disease comprising acquiring the level of a biomarker, e.g., leptin, creatine, adiponectin, an inflammatory cytokine (e.g., a pro-inflammatory cytokine, e.g., IL-6, MCP-1, or IL-23), glomerular filtration rate (GFR), a protein, or a structural abnormality. In some embodiments, the treatment comprises the administration of a MC4R agonist or pharmaceutically acceptable salt thereof. In some embodiments, the treatment comprises the administration of a compound of any one of Formulas (I), (II), (III), (IV), (V), (VI), (VII), (VIII), (X), or (XI), or a pharmaceutically acceptable salt thereof.

In some embodiments, the method further comprises comparing the acquired level of a biomarker with a reference value. In some embodiments, the comparing is responsive to the comparison administering the compound of any one of Formulas (I), (II), (III), (IV), (V), (VI), (VII), (VIII), (X), or (XI), or a pharmaceutically acceptable salt thereof. In some embodiments, a dosage or treatment of a compound of any one of Formulas (I), (II), (III), (IV), (V), (VI), (VII), (VIII), (X), or (XI) is administered responsive to the level of the biomarker.

In some embodiments, the biomarker is leptin. In some embodiments, the biomarker is creatine. In some embodiments, the biomarker is adiponectin. In some embodiments, the biomarker is glucose. In some embodiments, the biomarker is a salt, such as sodium, potassium, chloride, calcium, or phosphorus. In some embodiments, the biomarker is an inflammatory cytokine. In some embodiments, the inflammatory cytokine is a pro-inflammatory cytokine. In some embodiments, the pro-inflammatory cytokine comprises IL-6, MCP-1, or IL-23. In some embodiments, the biomarker is the level of protein in the urine. In some embodiments, the biomarker is the glomerular filtration rate (GFR). In some embodiments, the biomarker is a structural abnormality. In some embodiments, the subject has a structural abnormality in the kidney. In some embodiments, the structural abnormality comprises a fetal lobulation, a parenchymal cyst, a calyceal cyst, calyceal clubbing, or renal agenesia. In some embodiments, the subject is a mammal, e.g., a human.

In some embodiments, the compound is a compound of Formula (I) or a pharmaceutically acceptable salt thereof. In some embodiments, the compound of Formula (I) is Ac-Arg-c (Cys-D-Ala-His-D-Phe-Arg-Trp-Cys)-NH₂ (SEQ ID NO: 140). In some embodiments, the compound is a compound of Formula (II) or a pharmaceutically acceptable salt thereof. In some embodiments, the compound of Formula (II) is Hydantoin (C(O)-(Arg-Gly))-cyclo(Cys-Glu-His-D-Phe-Arg-Trp-Cys)-NH₂ (SEQ ID NO:13). In some embodiments, the compound of any one of Formulas (I), (II), (III), (IV), (V), (VI), (VII), (VIII), (X), or (XI) is formulated as a pharmaceutical composition.

The details of one or more embodiments of the disclosure are set forth herein. Other features, objects, and advantages of the disclosure will be apparent from the Detailed Description, the Figures, the Examples, and the claims.

### BRIEF DESCRIPTION OF THE DRAWINGS

FIG. 1A-B are graphs showing that setmelanotide treatment lowers plasma leptin levels (FIG. 1A) and renal IL-23 expression (FIG. 1B) in obese BBS10^{-/-} mice on HF/HG diet (as described in Example 1).

FIG. 2 is a chart showing that setmelanotide may improve renal dysfunction in BBS through 1) lowering leptin-induced inflammation and 2) reduction in body weight (as described in Example 2).

FIG. 3 is a flow chart depicting the study design outlined in Example 2.

FIG. 4 is a bar graph showing creatine clearance of BBS10^{-/-} mice before and after treatment with vehicle, peptide (setmelanotide), or pair-fed mice injected with vehicle.

FIGS. 5A-5C are bar graphs showing the blood plasma levels of leptin (FIG. 5A), adiponectin (FIG. 5B), and the leptin: adiponectin ratio in BBS10^{-/-} mice after treatment with vehicle, peptide (setmelanotide), or pair-fed mice injected with vehicle.

### DETAILED DESCRIPTION

This disclosure provides, at least in part, a method for treating chronic kidney disease in a subject in need thereof. In some embodiments, the method comprises administering a melanocoring 4 receptor (MC4R) agonist to the subject, e.g., a compound of any one of Formulas (I), (II), (III), (IV), (V), (VI), (VII), (VIII), (X), or (XI) described herein, or a pharmaceutically acceptable salt thereof.

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein “about” and “approximately” generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity, or a value, e.g., a numerical value, or knowledge of (e.g., knowledge of the sequence or mutational state of) a genotype or a nucleic acid or polypeptide, by “directly acquiring” or “indirectly acquiring” the physical entity, value, or knowledge. “Directly acquiring” means performing a physical process (e.g., performing a synthetic or analytical method) to obtain the physical entity, value, or knowledge. “Indirectly acquiring” refers to receiving the physical entity, value, or knowledge from another party or source (e.g., a third party laboratory that directly acquired the physical entity, value, or knowledge). Directly acquiring a value or knowledge includes performing a process that includes a physical change in a sample or another substance. Examples include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Administer”, “administering”, or “administration”, as used herein, refer to implanting, absorbing, ingesting, injecting, or otherwise introducing an entity described herein (e.g., a compound described herein, e.g., an MC4R agonist, e.g., a compound of any one of Formulas (I), (II), (III), (IV), (V), (VI), (VII), (VIII), (X), or (XI) or a pharmaceutically acceptable salt thereof).

“Early onset”, e.g., as in early onset obesity, as used herein, refers to an onset (e.g., first occurrence of one or more symptoms of a disorder, e.g., a disorder described herein, e.g., obesity) that occurs in a subject before adulthood, e.g., during childhood, e.g., when the subject is less 18 years of age or younger (e.g., 18, 17, 16, 15, 14, 13, 12, 11, 10, 9, 8, 7, 6, 5, 4, 3, 2, or 1 year of age or younger, or during adolescence, e.g., when the child is younger than 12 years of age or when the child is younger than 6 years of age).

As used herein, the term “mutation” refers to an altered nucleic acid sequence of a gene or fragment thereof compared to a wild-type sequence. For example, a mutation can include a point mutation, frame-shift mutation, missense mutation, inversion, deletion, insertion, truncation, chromosomal translocation. In embodiments, a mutation can result in the gene or fragment thereof coding for a non-functional protein, a protein with reduced activity (or a partially functional protein), or a protein with altered activity. For example, a “loss of function” mutation refers to a mutation that results in the gene or fragment thereof coding for a non-functional protein, which has substantially reduced activity compared to its wild-type counterpart (e.g., a non-functional protein has less than 10%, 9%, 8%, 7%, 6%, 5%, 4%, 3%, 2%, 1% or less activity than its wild-type counterpart). For example, “partial loss of function” mutation refers to a mutation that results in the gene or fragment thereof coding for a partially functional protein, which has reduced activity compared to its wild-type counterpart (e.g., a partially functional protein has less than 50% and greater than 10% of the activity of its wild-type counterpart).

“Prevention,” “prevent,” and “preventing” as used herein refers to a treatment that comprises administering or applying a therapy, e.g., administering a compound described herein, e.g., an MC4R agonist, e.g., a compound of any one of Formulas (I), (II), (III), (IV), (V), (VI), (VII), (VIII), (X), or (XI) or a pharmaceutically acceptable salt thereof, prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

“Subject” as used herein refers to a human or non-human animal. In an embodiment, the subject is a human (i.e., a male or female, e.g., of any age group, a pediatric subject (e.g., infant, child, adolescent) or adult subject (e.g., young adult, middle-aged adult, or senior adult)). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Unless defined otherwise, all technical and scientific terms used herein have the same meaning as commonly understood by one of ordinary skill in the art. The chemical structures and formulae set forth herein are constructed according to the standard rules of chemical valency known in the chemical arts. Also, all publications, patent applications, patents and other references mentioned herein are incorporated by reference in their entirety.

The nomenclature used to define the peptides is that typically used in the art wherein the amino group at the N-terminus appears to the left and the carboxyl group at the C-terminus appears to the right. Where the amino acid has D and L isomeric forms, it is the L form of the amino acid that is represented unless otherwise explicitly indicated.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The compounds useful for practicing the methods described herein may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the invention.

The compounds useful for practicing the methods described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” as used herein is meant to include salts of the active compounds that are prepared with relatively nontoxic acids or bases, depending on the particular substituents found on the compounds described herein. When compounds used in the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66:1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

The compounds useful for practicing the methods described herein can also exist in unsolvated forms as well as solvated forms, including hydrated forms. In general, the solvated forms are equivalent to unsolvated forms and are encompassed within the scope of the present disclosure. The compounds useful for practicing the methods described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of x electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

| Symbol | Meaning |
|---|---|
| Abu | α-aminobutyric acid |
| Ac | acyl group |
| Acc | 1-amino-1-cyclo(C₃-C₉)alkyl carboxylic acid |
| A3c | 1-amino-1cyclopropanecarboxylic acid |
| A4c | 1-amino-1-cyclobutanecarboxylic acid |
| A5c | 1-amino-1-cyclopentanecarboxylic acid |
| A6c | 1-amino-1-cyclohexanecarboxylic acid |
| Aha | 7-aminoheptanoic acid |
| Ahx | 6-aminohexanoic acid |
| Aib | α-aminoisobutyric acid |
| Aic | 2-aminoindan-2-carboxylic acid |
| Ala or A | Alanine |
| β-Ala | β-alanine |
| Apc | denotes the structure: |
| | 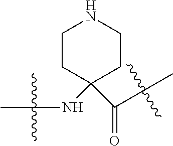 [SMILES: *NC1(C(*)=O)CCNCC1] |
| Apn | 5-aminopentanoic acid (HN—(CH2)₄—C(O) |
| Arg or R | Arginine |
| hArg | Homoarginine |
| Asn or N | Asparagine |
| Asp or D | aspartic acid |
| Bal | 3-benzothienylalanine |
| Bip | 4,4′-biphenylalanine, represented by the structure |
| | 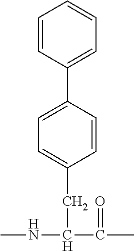 [SMILES: CNC(Cc1ccc(-c2ccccc2)cc1)C(C)=O] |
| Bpa | 4-benzoylphenylalanine |
| 4-Br-Phe | 4-bromo-phenylalanine |
| Cha | β-cyclohexylalanine |
| hCha | homo-cyclohexylalanine |
| Chg | Cyclohexylglycine |
| Cys or C | Cysteine |
| hCys | Homocysteine |
| Dab | 2,4-diaminobutyric acid |
| Dap | 2,3-diaminopropionic acid |
| Dip | β,β-diphenylalanine |
| Doc | 8-amino-3,6-dioxaoctanoic acid with the structure of: |
| | 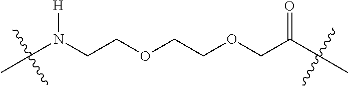 [SMILES: *NCCOCCOCC(*)=O] |
| 2-Fua | β-(2-furyl)-alanine |
| Gaba | 4-aminobutyric acid |
| Gln or Q | Glutamine |
| Glu or E | glutamic acid |
| Gly or G | Glycine |
| His or H | Histidine |
| 3-Hyp | trans-3-hydroxy-L-proline, i.e., (2S,3S)-3-hydroxy- |
| | pyrrolidine-2-carboxylic acid |
| 4-Hyp | 4-hydroxyproline, i.e., (2S,4R)-4-hydorxypyrrolidine- |
| | 2-carboxylic acid |
| Ile or 1 | Isoleucine |
| Leu or L | Leucine |
| hLeu | Homoleucine |
| Lys or K | Lysine |
| Met or M | Methionine |
| β-hMet | β-homomethionine |
| 1-Nal | β-(1-naphthyl)alanine |
| 2-Nal | β-(2-naphthyl)alanine |
| Nip | nipecotic acid |
| Nle | Norleucine |
| Ole | octahydroindole-2-carboxylic acid |
| Orn | Ornithine |
| 2-Pal | β-(2-pyridiyl)alanine |
| 3-Pal | β-(3-pyridiyl)alanine |
| 4-Pal | β-(4-pyridiyl)alanine |
| Pen | Penicillamine |
| Pff | (S)-pentafluorophenylalanine |
| Phe or F | Phenylalanine |
| hPhe | homophenylalanine |
| Pro or P | Proline |
| hProP | Homoproline |
| Ser or S | Serine |
| Tle | tert-Leucine |
| Taz | β-(4-thiazolyl)alanine |
| 2-Thi | β-(2-thienyl)alanine |
| 3-Thi | β-(3-thienyl)alanine |
| Thr or T | Threonine |
| Trp or W | Tryptopham |
| Tyr or Y | Tyrosine |
| D-(Et) Tyr | has a structure of |
| | 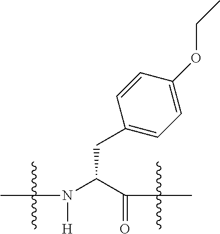 [SMILES: *N[C@H](Cc1ccc(OCC)cc1)C(*)=O] |
| Val or V | Valine |

Certain other abbreviations used herein are defined as follows:

| | |
|---|---|
| Boc: | tert-butyloxycarbonyl |
| Bzl: | Benzyl |
| DCM: | Dichloromethane |
| DIC: | N,N-diisopropylcarbodiimide |
| DIEA: | diisopropylethyl amine |
| Dmab: | 4-{N-(1-(4,4-dimethyl-2,6-dioxocyclohexylidene)-3- |
| | methylbutyl)-amino}benzyl |
| DMAP: | 4-(dimethylamino)pyridine |
| DMF: | Dimethylformamide |
| DNP: | 2,4-dinitrophenyl |
| Fm: | Fluorenylmethyl |
| Fmoc: | fluorenylmethyloxycarbonyl |
| For: | Formyl |
| HBTU: | 2-(1H-benzotriazole-1-yl)-1,1,3,3-tetramethyluronium |
| | hexafluorophosphate |
| cHex | Cyclohexyl |
| HOAT: | O-(7-azabenzotriazol-1-yl)-1,1,3,3-tetramethyluronium |
| | hexafluorophosphate |
| HOBt: | 1-hydroxy-benzotriazole |
| MBNA | 4-methylbenzhydrylamine |
| Mmt: | 4-methoxytrityl |
| NMP: | N-methylpyrrolidone |
| O-tBu | oxy-tert-butyl |
| Pbf: | 2,2,4,6,7-pentamethyldihydrobenzofuran-5-sulfonyl |
| PyBroP | bromo-tris-pyrrolidino-phosphonium hexafluorophosphate |
| tBu: | tert-butyl |
| TIS: | triisopropyIsilane |
| TOS: | Tosyl |
| Trt | Trityl |
| TFA: | trifluoro acetic acide |
| TFFH: | tetramethylfluoroforamidiaium hexafluorophosphate |
| Z: | benzyloxycarbonyl |

Unless otherwise indicated, with the exception of the N-terminal amino acid, all abbreviations (e.g. Ala) of amino acids in this disclosure stand for the structure of —NH—C(R)(R′)—CO—, wherein R and R′ each is, independently, hydrogen or the side chain of an amino acid (e.g., R═CH₃ and R′═H for Ala), or R and R′ may be joined to form a ring system.

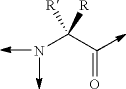

For the N-terminal amino acid, the abbreviation stands for the structure of:

The designation “NH₂” in e.g., as in Ac-Nle-c (Cys-D-Ala-His-D-Phe-Arg-Trp-Cys)-NH₂ (SEQ ID NO:13), indicates that the C-terminus of the peptide is amidated. Ac-Nle-c (Cys-D-Ala-His-D-Phe-Arg-Trp-Cys) (SEQ ID NO:107), or alternatively Ac-Nle-c (Cys-D-Ala-His-D-Phe-Arg-Trp-Cys)-OH (SEQ ID NO:107), indicates that the C-terminus is the free acid.

“-c (Cys-Cys)-” or “-cyclo(Cys-Cys)-” denotes the structure:

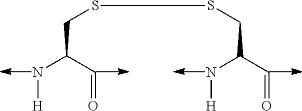

“-c (Cys-Pen)-” or “-cyclo(Cys-Pen)-” denotes the structure:

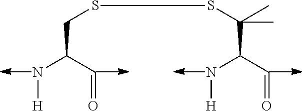

“-c (Asp-Lys)-” or “-cyclo(Asp-Lys)-” denotes the structure:

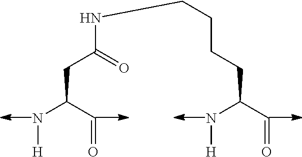 [SMILES: N[C@H](C=O)CCCCNC(=O)C[C@H](N)C=O]

The following abbreviations are used throughout the disclosure:

“Hydantoin-(C(O)-(A^{a}-A^{b}))” denotes the structure:

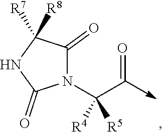

wherein amino acid “A^{a}” has the structure:

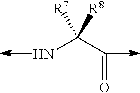

and amino acid “A^{b}” the structure:

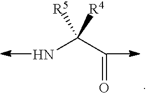

For example, “Hydantoin-(C(O)-Arg-A^{b}))” would have the following structure:

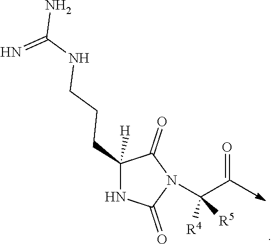

For example, “Hydantoin-(C(O)-(Arg-Gly))” would have the following structure:

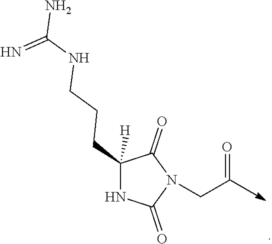 [SMILES: N=C(N)NCCC[C@@H]1NC(=O)N(CC=O)C1=O]

For example, a compound represented as “c [Hydantoin (C(O)-(Cys-A^{b}))-A¹-A²-A³-A⁴-Cys]-” would have the following the structure:

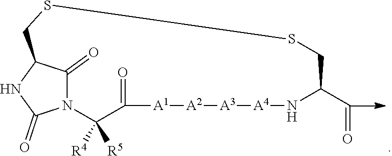

whereas a compound represented as “c [Hydantoin (C(O)-(A^{b}-Cys))-A¹-A²-A³-A⁴-Cys]-” would have the structure:

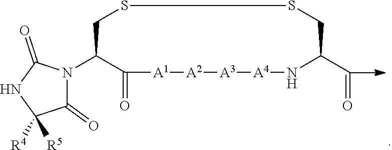

For further guidance, “c [Hydantoin (C(O)-(Asp-A^{b}))-A¹-A²-A³-A⁴-Lys]-” represents the following compound:

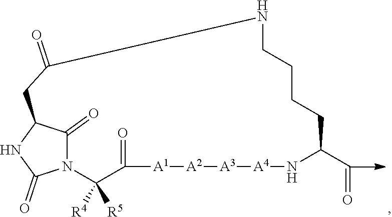

whereas “c [Hydantoin (C(O)-(Dap-A^{b}))-A¹-A²-A³-A⁴-Asp]-” has the following formula:

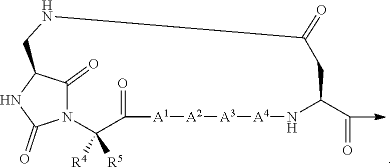

“Acyl” refers to R″—C(O)—, where R″ is H, alkyl, substituted alkyl, heteroalkyl, substituted heteroalkyl, alkenyl, substituted alkenyl, aryl, alkylaryl, or substituted alklyaryl, and is indicated in the general formula of a particular embodiment as “Ac”.

“Alkyl” refers to a hydrocarbon group containing one or more carbon atoms, where multiple carbon atoms if present are joined by single bonds. The alkyl hydrocarbon group may be straight-chain or contain one or more branches or cyclic groups.

“Hydroxyalkyl” refers to an alkyl group wherein one or more hydrogen atoms of the hydrocarbon group are substituted with one or more hydroxy radicals, such as hydroxymethyl, hydroxyethyl, hydroxypropyl, hydroxybutyl, hydroxypentyl, hydroxyhexyl and the like.

“Substituted alkyl” refers to an alkyl wherein one or more hydrogen atoms of the hydrocarbon group are replaced with one or more substituents selected from the group consisting of halogen, (i.e., fluorine, chlorine, bromine, and iodine), —OH, —CN, —SH, amine (e.g., —NH₂, —NHCH₃), —NO₂, guanidine, urea, amidine, and —C₁₋₂₀ alkyl, wherein said-C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present. The presence of —(CH₂)₀₋₂₀—COOH results in the production of an alkyl acid. Non-limiting examples of alkyl acids containing, or consisting of, —(CH₂)₀₋₂₀—COOH include 2-norbornane acetic acid, tert-butyric acid, 3-cyclopentyl propionic acid, and the like.

The term “halo” encompasses fluoro, chloro, bromo and iodo.

Guanidines are a group of organic compounds that share a common functional group with the general structure (R¹R²N)(R³R⁴N)C═N—R⁵. The central bond within this group is an imine, and the group is related structurally to amidines and ureas.

“Heteroalkyl” refers to an alkyl wherein one of more of the carbon atoms in the hydrocarbon group is replaced with one or more of the following groups: amino, amido, —O—, —S— or carbonyl. In different embodiments 1 or 2 heteroatoms are present.

“Substituted heteroalkyl” refers to a heteroalkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, —NO₂, and —C₁₋₂₀ alkyl, wherein said-C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.

“Alkenyl” refers to a hydrocarbon group made up of two or more carbons where one or more carbon-carbon double bonds are present. The alkenyl hydrocarbon group may be straight-chain or contain one or more branches or cyclic groups.

“Substituted alkenyl” refers to an alkenyl wherein one or more hydrogens are replaced with one or more substituents selected from the group consisting of halogen (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.

“Aryl” refers to an optionally substituted aromatic group with at least one ring having a conjugated pi-electron system, containing up to three conjugated or fused ring systems. Aryl includes carbocyclic aryl, heterocyclic aryl and biaryl groups. Preferably, the aryl is a 5- or 6-membered ring. Preferred atoms for a heterocyclic aryl are one or more sulfur, oxygen, and/or nitrogen. Non-limiting examples of aryl include phenyl, 1-naphthyl, 2-naphthyl, indole, quinoline, 2-imidazole, 9-anthracene, and the like. Aryl substituents are selected from the group consisting of —C₁₋₂₀ alkyl, —C₁₋₂₀ alkoxy, halogen (i.e., fluorine, chlorine, bromine, and iodine), —OH, —CN, —SH, —NH₂, —NO₂, —C₁₋₂₀ alkyl substituted with halogens, —CF₃, —OCF₃, and —(CH₂)₀₋₂₀—COOH. In different embodiments the aryl contains 0, 1, 2, 3, or 4 substituents.

“Alkylaryl” refers to an “alkyl” joined to an “aryl”.

The term “(C₁₋₁₂) hydrocarbon moiety” encompasses alkyl, alkenyl and alkynyl and in the case of alkenyl and alkynyl there is C₂-C₁₂.

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

Designation “(amino acid),” means that an amino acid is repeated n times. For example, designation “(Pro)₂” or “(Arg)₃” mean that proline or arginine residues are repeated, respectively, two or three times.

### Disorders

Described herein are methods for treating chronic kidney disease in a with an MC4R agonist, e.g., a compound of any one of Formulas (I), (II), (III), (IV), (V), (VI), (VII), (VIII), (X), or (XI) or a composition thereof. In some embodiments, the methods described herein directly or indirectly reduce or alleviate at least one symptom of chronic kidney disease. In some embodiments, the methods described herein prevent or slow the onset of chronic kidney disease. In some embodiments, the subject may have a co-morbidity, such as obesity, Bardet-Biedl syndrome (BBS), Alstrom syndrome, polycystic kidney disease (e.g., dominant (ADPKD for autosomal dominant polycystic kidney disease) or recessive (ARPKD for autosomal recessive polycystic kidney disease)), Joubert syndrome, Meckel-Gruber syndrome, or orofaciodigital syndrome 1. In some embodiments, the subject is a human.

### Chronic Kidney Disease

Chronic kidney disease (CKD) refers to a type of kidney disease resulting in gradual loss of kidney function over an extended period of time. CKD affects over 320 million people worldwide, resulting in 1.2 million deaths annually. CKD may be caused by a number of conditions, including diabetes, high blood pressure, vascular disease (e.g., bilateral renal artery stenosis, ischemic nephropathy, hemolytic-uremic syndrome, and vasculitis), glomerular disease (e.g., primary glomerular disease or secondary glomerular disease), fatigue, medication use, obstructive nephropathy (e.g., kidney stones, tumor), or an infection (e.g., pinworm infection).

In general, a subject with chronic kidney disease initially presents without detectable signs or symptoms. However, as the kidney function declines over time, a subject may experience one or more of the following symptoms: high blood pressure, accumulation of urea (e.g., azotemia or uremia), hyperkalemia, decrease in erythropoietin synthesis, edema, iron deficiency anemia, metabolic acidosis, chronic kidney disease-mineral bone disorder, hypocalcemia, calciphylaxis, hyperphosphatemia, atherosclerosis, cardiovascular disease, and sexual dysfunction. Numerous uremic toxins accumulate in the blood of a CKD patient, which may serve as a biomarker for the disease. Exemplary uremic toxins include urea, 2-heptenal, 2-hexenal, 2-nonenal, 2-octenal, 4-decanal, anthranilic acid, argininic acid, cysteine, and dimethylamine (see, e.g., Vandolder, R. et al (2003) Kidney Intl 5:1934-1943 and Duranton, F. J Am Soc Nephrol (2012)13:1258-1270; each of which is incorporated herein by reference).

A subject may be diagnosed with CKD by blood test and/or urine test. A blood test may include measurement of the glomerular filtration rate (GFR), which represents the volume of fluid filtered from the renal glomerular capillaries to the Bowman's capsule per unit time. The GFR may be dependent on the difference between the higher blood pressure created by vasoconstriction of the input or afferent arteriole versus the lower blood pressure created by lesser vasoconstriction of the output or efferent arteriole. As the GFR decreases, the prognosis of the subject worsens. A GFR measurement of between 100-120 mL/min typically represents a normal, healthy GFR. A GFR measurement of less than 60 mL/min may indicate that uremic symptoms are present, while a GFR between 30-60 mL/min frequently leads to cognitive impairment. GFR measurements below 50 mL/min result in likely insulin resistance, and a GFR equal to or less than 15 mL/min corresponds to kidney failure. In some embodiments, a subject has a GFR less than about 120 mL/min, 110 mL/min, 100 mL/min, 90 mL/min, 80 mL/min, 70 mL/min, 60 mL/min, 50 mL/min, 40 mL/min, 30 mL/min, or 20 mL/min.

A subject having CKD may also exhibit proteinuria, in which a higher level of protein is present in the urine (e.g., albumin) of the subject compared with a reference level. Proteinuria is often diagnosed through urine analysis, which compares the level of albumin in the urine with the amount of creatine in the urine (e.g., providing the urine albumin to creatine ratio (UACR)). A UACR over 30 mg/g is often considered the threshold for CKD. In some embodiments, a subject is has a UACR over 30 mg/g. In some embodiments, the subject has a UACR of more than 35 mg/g or more than 50 mg/g. In some embodiments, a subject having CKD has a higher level of protein in the urine compared with a reference value (e.g., the level of protein in a subject not having CKD). In some embodiments, a subject having CKD has at least a 1%, 2.5%, 5%, 10%, 15%, 20%, 25%, 30%, 35%, 40%, 45%, 50%, 55%, 60%, 65%, 70%, or 75% higher level of protein in the urine compared with a reference value.

In some embodiments, the CKD comprises end-stage renal failure. In some embodiments, the CKD is non-dialysis dependent. In some embodiments, the CKD is dialysis-dependent or requires kidney transplantation.

### Obesity

In some embodiments, a subject having chronic kidney disease may also be obese. Obesity refers to a condition in which a subject having a body mass index (BMI) within the ranges defined as obese by the Center for Disease Control (see, e.g., cdc.gov/obesity/defining.html and www.cdc.gov/obesity/childhood/defining.html) or as defined by “Clinical Guidelines on the Identification, Evaluation, and Treatment of Overweight and Obesity in Adults” from the National Institutes of Health. BMI is obtained by dividing a subject's weight, e.g., in kilograms (kg) by the square of the subject's height, e.g., in meter (m). For example, an adult who has a BMI of 30 kg/m² or higher is considered obese. For example, an adult with a BMI of 25.0 to 29.9 kg/m² is considered overweight; an adult with a BMI of 18.5 to 24.9 kg/m² is considered to have a normal or healthy weight range; and an adult with a BMI of less than 18.5 kg/m² is considered to be underweight. For example, an adult having a height of 5 feet, 9 inches with a body weight of 203 pounds or more is considered obese. For children and teens, obese refers to a subject having a BMI at or above the 85th to 95th percentile for children and teens of the same age and sex.

Obesity, diabetes and hypertension are also likely contributors to CKD manifestation and progression (Stenvinkel, P., et al (2013). J Am Soc Nephrol 24, 1727-1736).

In some embodiments, a subject may be considered severely obese. A subject considered severely obese has a BMI of 35 kg/m² or higher, e.g., 40 kg/m² or higher. For example, a severely obese subject is over 100% over the ideal (normal, healthy) body weight.

In some embodiments, a subject may further have hyperphagia.

### Bardet-Biedl Syndrome

In embodiments, a subject having chronic kidney disease may also have Bardet-Biedl syndrome (BBS). BBS is an autosomal recessive ciliopathy characterized by a panoply of clinical symptoms and tissue pathologies, including obesity, cognitive impairment, retinal degeneration and renal dysfunction. BBS is a form of Laurence-Moon-Beidl syndrome and is characterized by obesity, retinopathy, learning disability, polydactyly, and hypogenitalism (See, e.g., Green et al. New Engl. J. Med. 321 (1989): 1002-9). Without wishing to be bound by theory, it is believed that BBS is characterized by one or more mutation(s) in one or more of 20 genes (BBS1-BBS20). Most of the BBS genes encode proteins thought to be important for the function, formation, and stability of cilia. It is believed that eight BBS proteins (BBS1, BBS2, BBS4, BBS5, BBS7, BBS8, BBS9, and BBS18) form a complex called the BBSome that mediates trafficking to the ciliary membrane. BBS6, BBS10, and BBS12 are believed to form a complex with the CCT/TRIC family of group II chaperonins.

Mutation(s) in the BBS gene(s) are thought to lead to defective cilia, e.g, neuronal cilia, or dysfunctional ciliary regulation. Ciliary dysfunction is believed to cause impaired leptin signaling and hyperleptinemia. The role of primary cilia and cilia proteins in energy homeostasis and obesity-related disorders is described, e.g., in Gupta et al. J. Endocrinol. 203 (2009): 327-36; and Oh et al. Cell Metab. 21.1 (2015): 21-31. Patients with BBS have been found to have hyperleptinemia that is suggestive of leptin resistance, with triglycerides, leptin, diastolic BP—Z, and intra-abdominal fat mass significantly greater in BBS patients than in controls (see, e.g., Feuillan et al. J. Clin. Endocrinol. Metab. 96.3 (2011)). Obesity in BBS mutant mice, for example, is thought to be caused by leptin resistance and defects in leptin receptor trafficking (see, e.g., Berbari et al. Proc. Natl. Acad. Sci. USA 110.19 (2013): 7796-7801). BBS2, BB4, and BB6 mutant mice have been shown to be hyperleptinemic and failed to reduce their food intake in response to leptin (see, e.g., Berbari et al. Proc. Natl. Acad. Sci. USA 110.19 (2013): 7796-7801).

Renal involvement is evident in 53-82% of subjects with BBS (Forsythe, E. et al. (2017). J Am Soc Nephrol 28, 963-970) and can present in various forms-structural abnormalities are commonly observed (fetal lobulations, parenchymal cysts, calyceal cysts and clubbing, and renal agenesia) but are not always associated with functional impairment (O'Dea, D., et al (1996). Am J Kidney Dis 27, 776-783). Statistics on the incidence and severity of renal dysfunction in BBS are highly variable, however in the largest single study (n=350), renal insufficiency leading to chronic kidney disease has been observed in approximately half of BBS patients (45% in children and 46% in adults), stage IV-V CKD was evident in 6% of children and 8% of adults (Forsythe et al 2017). End-stage renal disease (ESRD) is a predominant cause of morbidity and mortality in BBS individuals with CKD with renal involvement indicated in 75% of BBS deaths (O'Dea et al 1996). Glomerulopathy and urine concentrating defects are common early clinical presentations with patients exhibiting reduced glomerular filtration rates (GFR), elevated UACR ratio and polyuria. The pathogenic mechanism underlying renal insufficiency in BBS remains to be determined, however it may be influenced by genotype since BBS10 and BBS12 patients exhibit greater renal dysfunction than BBS1 patients (Forsythe et al 2015 & 2017). Severity of renal involvement also differs in mouse modes of BBS (Guo, D. F., et al (2011). Am J Physiol Renal Physiol 300, F574-580). Renal transplantation is a viable and successful treatment option for BBS patients with ESRD, although body weight increase is a significant iatrogenic sequela (Haws, R. M., et al (2016). Pediatr Nephrol 31, 2153-2161).

In some embodiments, the subject has been diagnosed with BBS. In some embodiments, the subject is at risk for having BBS. In some embodiments, the subject does not have BBS. In some embodiments, the subject has not been diagnosed with BBS.

Despite the prevalence of obesity, BBS patients exhibit hyperleptinemia that is disproportionate to their adiposity (Feuillan et al 2011). Furthermore, CRP levels are significantly elevated in BBS10>BBS1 patients, consistent with the severity of renal pathology and independently of WBC and BMI. Based upon clinical epidemiological data in non-BBS related CKD it is possible that these pro-inflammatory markers contribute to the severity/progression of CKD in BBS, although this is yet to be clinically established. In support of this notion, recent pre-clinical work by Vincent Marion (Zacchia et al Unpub.) indicates that hyperleptinemia in mouse models of BBS drives a body weight-related pro-inflammatory program that leads to progressive renal dysfunction. Specifically, obese BBS12^{-/-} mice on HF/HG diet or non-obese BBS12^{-/-} injected with leptin (to the same plasma concentration) exhibited reduced eGFR, systemic inflammation (IL-6 and MCP-1), renal up-regulation of pro-inflammatory markers (IL-6, MCP-1, IL-23), increased monocyte infiltration, increased apoptosis and glomerular morphological defects. Furthermore, these effects were abrogated by podocyte deletion of LepR, indicating a direct mechanism of action. In sum, mouse models of BBS exhibit impaired renal function in keeping with the clinical condition and hyperleptinemia drives progressive CKD.

### Alström Syndrome

Alström syndrome (ALMS) is an autosomal recessive disease with clinical symptoms that include severe obesity, hyperinsulinemia, and altered glucose metabolism that can lead to the development of type 2 diabetes at a young age in afflicted subjects. ALMS is caused by mutations in ALMS1, a gene that has been mapped to chromosome 2p13.

The progression from early onset obesity toward the impaired fasting glucose or impaired glucose tolerance and overt diabetes is believed to occur mostly because of a progressive failure of β-cell insulin secretion without any further worsening of insulin resistance with age, even in the presence of weight reduction (see, e.g., Bettini et al. Pediatr. Diabetes 13:59-67, 2012).

In some embodiments, the subject having chronic kidney disease has Alström syndrome (ALMS). In some embodiments, the subject has been diagnosed with ALMS. In some embodiments, the subject is at risk for having ALMS. In some embodiments, the subject does not have ALMS. In some embodiments, the subject has not been diagnosed with ALMS.

### Other Conditions

In some embodiments, a subject having chronic kidney disease may also have polycystic kidney disease. Polycystic kidney disease (PKD) is an inherited disorder in which clusters of cysts develop primarily within the kidneys, causing the kidneys to enlarge and lose function over time. Cysts may be noncancerous round sacs containing fluid. The cysts may vary in size, and may grow very large. Polycystic kidney disease can result in high blood pressure and kidney failure in a subject. Other symptoms include back or side pain, headache, a feeling of fullness in the abdomen, increased size of the abdomen, blood in urine, kidney stones and urinary tract or kidney infections. Polycystic kidney disease may comprise dominant (ADPKD for autosomal dominant polycystic kidney disease) or recessive (ARPKD for autosomal recessive polycystic kidney disease). In some embodiments, the subject has been diagnosed with polycystic kidney disease. In some embodiments, the subject is at risk for having polycystic kidney disease. In some embodiments, the subject does not have polycystic kidney disease. In some embodiments, the subject has not been diagnosed with polycystic kidney disease.

In some embodiments, a subject having chronic kidney disease may also have Joubert syndrome. Joubert syndrome is a disorder of brain development that may affect many parts of the body. It is characterized by the absence or underdevelopment of the cerebellar vermis (a part of the brain that controls balance and coordination) and a malformed brain stem (connection between the brain and spinal cord). In some embodiments, the subject has been diagnosed with Joubert syndrome. In some embodiments, the subject is at risk for having Joubert syndrome. In some embodiments, the subject does not have Joubert syndrome. In some embodiments, the subject has not been diagnosed with Joubert syndrome.

In some embodiments, a subject having chronic kidney disease may also have Meckel-Gruber syndrome. Meckel-Gruber syndrome is a rare, ciliopathic genetic disorder, characterized by renal cystic dysplasia, central nervous system malformations (occipital encephalocele), polydactyly (post axial), hepatic developmental defects, and pulmonary hypoplasia due to oligohydramnios. Dysplastic kidneys are prevalent in over 95% of all identified cases. When this occurs, microscopic cysts develop within the kidney and slowly destroy it, causing it to enlarge to 10 to 20 times its original size. Occipital encephalocele is present in 60% to 80% of all cases, and post-axial polydactyly is present in 55% to 75% of the total number of identified cases. Bowing or shortening of the limbs are also common. In some embodiments, the subject has been diagnosed with Meckel-Gruber syndrome. In some embodiments, the subject is at risk for having Meckel-Gruber syndrome. In some embodiments, the subject does not have Meckel-Gruber syndrome. In some embodiments, the subject has not been diagnosed with Meckel-Gruber syndrome.

In some embodiments, a subject having chronic kidney disease may also have orofaciodigital syndrome 1. Orofaciodigital syndrome 1 is a condition that affects the development of the oral cavity (the mouth and teeth), facial features, and digits (fingers and toes). This condition also causes polycystic kidney disease. Orofaciodigital syndrome 1 is caused by a change (mutation) in a gene called OFD1 which appears to play an important role in the early development of many parts of the body including the brain, face, limbs, and kidneys. [1] The syndrome is inherited in an X-linked dominant pattern. The diagnosis of OFD1 is sometimes made at birth, but it may be suspected only after polycystic kidney disease is found in later childhood or adulthood. Treatment for OFD1 typically focuses on the symptoms an individual has and may include surgery for cleft lip or palate, other oral abnormalities, or syndactyly (webbing of the fingers or toes). At least 13 potential forms of orofaciodigital syndromes have been identified, which are classified by their patterns of signs and symptoms. OFD1 is the most common form of orofaciodigital syndrome and differs from the other types mainly by its association with polycystic kidney disease. In some embodiments, the subject has been diagnosed with orofaciodigital syndrome 1. In some embodiments, the subject is at risk for having orofaciodigital syndrome 1. In some embodiments, the subject does not have orofaciodigital syndrome 1. In some embodiments, the subject has not been diagnosed with orofaciodigital syndrome 1.

### Compounds

Described herein are methods for the treatment of chronic kidney disease comprising administering to a subject a melanocorin 4 receptor (MC4R) agonist. Examples of naturally occurring MC4R agonists include α-MSH, β-MSH, γ-MSH and adenocorticitropic hormone (ACTH) or a functional fragment thereof. Examples of synthetic MC4R agonists are described in detail below.

In some embodiments, an MC4R agonist can be any known agonist of MC4R. In some example embodiment, the MC4R agonist is not an adrenocorticotropic hormone (ACTH) or a fragment thereof. Exemplary MC4R agonists include those described in WO2011104378; WO2011104379; WO201060901; WO200887189, WO200887188, WO200887187, WO200887186; US20110065652; WO2010144341; WO2010144344; WO201065799; WO201065800; WO201065801; WO201065802; WO201037081; WO2009152079; WO2009151383; US20100311648; US20100280079; WO201081666; WO201034500; WO200910299; WO2008116665; WO201052256; WO201052255; WO201126015; US20100120783; WO201096854; US20100190793; WO201025142; and WO201015972. Further examples of MC4R agonists are found in U.S. Pat. Nos. 8,263,608; 8,247,530; 8,114,844; and 7,968,548. The entire teachings of these publications are incorporated herein by reference.

In some embodiments, the MC4R agonist is a compound of any one of Formulas (I), (II), (III), (IV), (V), (VI), (VII), (VIII), (X), or (XI), or a pharmaceutically acceptable salt thereof as described herein. In some embodiments, the MC4R agonist is a compound of any one of Formulas (I) or (II), or a pharmaceutically acceptable salt thereof as described herein. In one embodiment, the MC4R agonist is a compound of Formula (I). In one embodiment, the MC4R agonist is a compound of Formula (II).

In some embodiments, the MC4R agonist is a compound of Formula (I):

(R²R³)-A¹-c(A²-A³-A⁴-A⁵-A⁶-A⁷-A⁸-A⁹)-A¹⁰-R¹ (I)

or a pharmaceutically acceptable salt thereof, wherein:
A¹ is Acc, HN—(CH₂)ₘ—C(O), L- or D-amino acid, or deleted;
A² is Cys, D-Cys, hCys, D-hCys, Pen, D-Pen, Asp, or Glu;
A³ is Gly, Ala, β-Ala, Gaba, Aib, D-amino acid, or deleted;
A⁴ is His, 2-Pal, 3-Pal, 4-Pal, Taz, 2-Thi, 3-Thi, or (X¹, X², X³, X⁴, X⁵) Phe;
A⁵ is D-Phe, D-1-Nal, D-2-Nal, D-Trp, D-Bal, D-(X¹, X², X³, X⁴, X⁵) Phe, L-Phe or D-(Et) Tyr;
A⁶ is Arg, hArg, Dab, Dap, Lys, Orn, or HN—CH((CH₂)ₙ—N(R⁴ R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂)ₛ—C(O), or deleted;
A⁹ is Cys, D-Cys, hCys, D-hCys, Pen, D-Pen, Dab, Dap, Orn, or Lys;
A¹⁰ is Acc, HN—(CH₂)ₜ—C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R4 and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂-10)alkenyl, substituted (C₂-10)alkenyl, (C₂₋₁₀)alkynyl, substituted (C₂₋₁₀)alkynyl, aryl, substituted aryl, OH, NH₂, NO₂, or CN.

In some embodiments, for Formula (I), when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or —C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl.

In some embodiments, for Formula (I), when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;

In some embodiments, for Formula (I), either A³ or A⁸ or both must be present in said compound.

In some embodiments, for Formula (I) when A² is Cys, D-Cys, hCys, D-hCys, Pen, or D-Pen, then A⁹ is Cys, D-Cys, hCys, D-hCys, Pen, or D-Pen.

In some embodiments, for Formula (I), when A² is Asp or Glu, then A⁹ is Dab, Dap, Orn, or Lys.

In some embodiments, for Formula (I), when A⁸ is Ala or Gly, then A¹ is not NIe.

In some embodiments, for Formula (I), when A¹ is deleted, then R² and R³ cannot both be H.

In some embodiments, for Formula (I): A¹ is A⁶c, Arg, D-Arg, Cha, D-Cha, hCha, Chg, D-Chg, Gaba, Ile, Leu, hLeu, Met, β-hMet, 2-Nal, D-2-Nal, Nip, Nle, Oic, Phe, D-Phe, hPhe, hPro, Val, or deleted; A² is Asp, Cys, D-Cys, hCys, D-hCys, Glu, Pen, or D-Pen; A³ is D-Abu, Aib, Ala, β-Ala, D-Ala, D-Cha, Gaba, D-Glu, Gly, D-Ile, D-Leu, D-Tle, D-Val, or deleted; A⁴ is His or 3-Pal; A⁵ is D-Bal, D-1-Nal, D-2-Nal, D-Phe, D-Trp, or D-(Et) Tyr; A⁶ is Arg, or hArg; A⁷ is Bal, Bip, 1-Nal, 2-Nal, Trp, D-Trp; A⁸ is A⁶c, D-Ala, Aha, Ahx, Ala, β-Ala, Apn, Gaba, Gly or deleted; A⁹ is Cys, D-Cys, hCys, D-hCys, Lys, Pen, or D-Pen; and A¹⁰ is Thr, or deleted, wherein at least one of A³ or A⁸ is deleted, but not both.

In some embodiments, the compound of Formula (I) is a compound disclosed in International Patent Application Publication Number WO 2007/008704, which is incorporated herein by reference in its entirety.

In some embodiments, the compound of Formula (I) is selected from:

SEQ ID NO: 1
Ac-Nle-c(Asp-His-D-Phe-Arg-Trp-β-Ala-Lys)-NH₂;
SEQ ID NO: 2
Ac-Nle-c(Asp-His-D-Phe-Arg-Trp-A6c-Lys)-NH₂;
SEQ ID NO: 3
Ac-Nle-c(Cys-His-D-Phe-Arg-Trp-Ahx-Cys)-NH₂;
SEQ ID NO: 4
D-Phe-c(Cys-His-D-Phe-Arg-Trp-Ala-D-Cys)-Thr-NH₂;
SEQ ID NO: 5
D-Phe-c(Cys-His-D-Phe-Arg-Trp-β-Ala-D-Cys)-Thr-NH₂;
SEQ ID NO: 6
D-Phe-c(Cys-His-D-Phe-Arg-Trp-Gaba-D-Cys)-Thr-NH₂;
SEQ ID NO: 7
Ac-Nle-c(Cys-His-D-Phe-Arg-Trp-Apn-Cys)-NH₂;
SEQ ID NO: 8
Ac-Nle-c(Asp-His-D-Phe-Arg-Trp-Apn-Lys)-NH₂;
SEQ ID NO: 9
Ac-A6c-c(Asp-His-D-Phe-Arg-Trp-Gaba-Lys)-NH₂;
SEQ ID NO: 10
Ac-D-2-Nal-c(Asp-His-D-Phe-Arg-Trp-Gaba-Lys)-NH₂;
SEQ ID NO: 11
Ac-Cha-c(Asp-His-D-Phe-Arg-Trp-Gaba-Lys)-NH₂;
SEQ ID NO: 12
Ac-Nle-c(Asp-His-D-Phe-Arg-Trp-Gaba-Lys)-NH₂;
SEQ ID NO: 13
Ac-Nle-c(Cys-D-Ala-His-D-Phe-Arg-Trp-Cys)-NH₂;
SEQ ID NO: 14
Ac-Nle-c(Cys-β-Ala-His-D-Phe-Arg-Trp-Cys)-NH₂;
SEQ ID NO: 15
Ac-Nle-c(Cys-Gaba-His-D-Phe-Arg-Trp-Cys)-NH₂;
SEQ ID NO: 16
Ac-Nle-c(Cys-Aib-His-D-Phe-Arg-Trp-Cys)-NH₂;
SEQ ID NO: 17
Ac-Nle-c(Cys-Gly-His-D-Phe-Arg-Trp-Cys)-NH₂;
SEQ ID NO: 18
Ac-Nle-c(D-Cys-Ala-His-D-Phe-Arg-Trp-Cys)-NH₂;
SEQ ID NO: 19
Ac-Nle-c(D-Cys-D-Ala-His-D-Phe-Arg-Trp-Cys)-NH₂;
SEQ ID NO: 20
Ac-Nle-c(D-Cys-β-Ala-His-D-Phe-Arg-Trp-Cys)-NH₂;
SEQ ID NO: 21
Ac-Nle-c(D-Cys-Gaba-His-D-Phe-Arg-Trp-Cys)-NH₂;
SEQ ID NO: 22
Ac-Nle-c(D-Cys-Aib-His-D-Phe-Arg-Trp-Cys)-NH₂;
SEQ ID NO: 23
Ac-Nle-c(D-Cys-Gly-His-D-Phe-Arg-Trp-Cys)-NH₂;
SEQ ID NO: 24
Ac-Nle-c(Cys-D-Ala-His-D-Phe-Arg-Trp-D-Cys)-NH₂;
SEQ ID NO: 25
Ac-Nle-c(Cys-β-Ala-His-D-Phe-Arg-Trp-D-Cys)-NH₂;
SEQ ID NO: 26
Ac-Nle-c(Cys-Gaba-His-D-Phe-Arg-Trp-D-Cys)-NH₂;
SEQ ID NO: 27
Ac-Nle-c(Cys-Aib-His-D-Phe-Arg-Trp-D-Cys)-NH₂;
SEQ ID NO: 28
Ac-Nle-c(Cys-Gly-His-D-Phe-Arg-Trp-D-Cys)-NH₂;
SEQ ID NO: 29
Ac-Nle-c(D-Cys-Ala-His-D-Phe-Arg-Trp-D-Cys)-NH₂;
SEQ ID NO: 30
Ac-Nle-c(D-Cys-D-Ala-His-D-Phe-Arg-Trp-D-Cys)-NH₂;
SEQ ID NO: 31
Ac-Nle-c(D-Cys-β-Ala-His-D-Phe-Arg-Trp-D-Cys)-NH₂;
SEQ ID NO: 32
Ac-Nle-c(D-Cys-Gaba-His-D-Phe-Arg-Trp-D-Cys)-NH₂;
SEQ ID NO: 33
Ac-Nle-c(D-Cys-Aib-His-D-Phe-Arg-Trp-D-Cys)-NH₂;
SEQ ID NO: 34
Ac-Oic-c(Asp-His-D-Phe-Arg-Trp-Gaba-Lys)-NH₂;
SEQ ID NO: 35
Ac-Chg-c(Asp-His-D-Phe-Arg-Trp-Gaba-Lys)-NH₂;
SEQ ID NO: 36
Ac-hCha-c(Asp-His-D-Phe-Arg-Trp-Gaba-Lys)-NH₂;
SEQ ID NO: 37
Ac-D-Cha-c(Asp-His-D-Phe-Arg-Trp-Gaba-Lys)-NH₂;
SEQ ID NO: 38
Ac-D-hCha-c(Asp-His-D-Phe-Arg-Trp-Gaba-Lys)-NH₂;
SEQ ID NO: 39
Ac-Nip-c(Asp-His-D-Phe-Arg-Trp-Gaba-Lys)-NH₂;
SEQ ID NO: 40
Ac-hPro-c(Asp-His-D-Phe-Arg-Trp-Gaba-Lys)-NH₂;
SEQ ID NO: 41
Ac-hLeu-c(Asp-His-D-Phe-Arg-Trp-Gaba-Lys)-NH₂;
SEQ ID NO: 42
Ac-Phe-c(Asp-His-D-Phe-Arg-Trp-Gaba-Lys)-NH₂;
SEQ ID NO: 43
Ac-D-Phe-c(Asp-His-D-Phe-Arg-Trp-Gaba-Lys)-NH₂;
SEQ ID NO: 44
Ac-D-Chg-c(Asp-His-D-Phe-Arg-Trp-Gaba-Lys)-NH₂;
SEQ ID NO: 45
n-butanoyl-Cha-c(Asp-His-D-Phe-Arg-Trp-Gaba-Lys)-NH₂;
SEQ ID NO: 46
n-butyryl-Cha-c(Asp-His-D-Phe-Arg-Trp-Gaba-Lys)-NH₂;
SEQ ID NO: 47
Ac-hPhe-c(Asp-His-D-Phe-Arg-Trp-Gaba-Lys)-NH₂;
SEQ ID NO: 48
Ac-β-hMet-c(Asp-His-D-Phe-Arg-Trp-Gaba-Lys)-NH₂;
SEQ ID NO: 49
Ac-Gaba-c(Asp-His-D-Phe-Arg-Trp-Gaba-Lys)-NH₂;
SEQ ID NO: 50
Ac-Cha-c(Asp-His-D-Phe-Arg-D-Trp-Ala-Lys)-NH₂;
SEQ ID NO: 51
Ac-hCha-c(Asp-His-D-Phe-Arg-D-Trp-Ala-Lys)-NH₂;
SEQ ID NO: 52
Ac-Leu-c(Asp-His-D-Phe-Arg-D-Trp-Ala-Lys)-NH₂;
SEQ ID NO: 53
Ac-hLeu-c(Asp-His-D-Phe-Arg-D-Trp-Ala-Lys)-NH₂;
SEQ ID NO: 54
Ac-Phe-c(Asp-His-D-Phe-Arg-D-Trp-Ala-Lys)-NH₂;
SEQ ID NO: 55
Ac-Nle-c(Asp-His-D-Phe-Arg-D-Trp-D-Ala-Lys)-NH₂;
SEQ ID NO: 56
Ac-Nle-c(Asp-His-D-Phe-Arg-D-Trp-P-Ala-Lys)-NH₂;
SEQ ID NO: 57
Ac-Nle-c(Asp-His-D-Phe-Arg-D-Trp-Gaba-Lys)-NH₂;
SEQ ID NO: 58
Ac-Nle-c(Asp-His-D-Phe-Arg-D-Trp-Aha-Lys)-NH₂;
SEQ ID NO: 59
Ac-Nle-c(Asp-His-D-Phe-Arg-D-Trp-Apn-Lys)-NH₂;
SEQ ID NO: 60
Ac-Nle-c(Cys-His-D-Phe-Arg-D-Trp-Apn-Cys)-NH₂;
SEQ ID NO: 61
Ac-Nle-c(Cys-His-D-Phe-Arg-D-Trp-Gaba-Cys)-NH₂;
SEQ ID NO: 62
Ac-Nle-c(Cys-His-D-Phe-Arg-D-Trp-Ahx-Cys)-NH₂;
SEQ ID NO: 63
Ac-Nle-c(Cys-His-D-Phe-Arg-D-Trp-β-Ala-Cys)-NH₂;
SEQ ID NO: 64
Ac-Nle-c(Cys-His-D-Phe-Arg-D-Trp-D-Ala-Cys)-NH₂;
SEQ ID NO: 65
Ac-Nle-c(Cys-D-Ala-His-D-2-Nal-Arg-Trp-Cys)-NH₂;
SEQ ID NO: 66
Ac-Nle-c(Cys-D-Ala-His-D-2-Nal-Arg-2-Nal-Cys)-NH₂;
SEQ ID NO: 67
Ac-Nle-c(Cys-D-Ala-His-D-2-Nal-Arg-1-Nal-Cys)-NH₂;
SEQ ID NO: 68
n-butanoyl-Nle-c(Cys-D-Ala-His-D-Phe-Arg-2-Nal-Cys)-NH₂;
SEQ ID NO: 69
n-butanoyl-Nle-c(Cys-D-Ala-His-D-Phe-Arg-Trp-Cys)-NH₂;
SEQ ID NO: 70
Ac-Nle-c(Cys-D-Ala-His-D-Phe-Arg-2-Nal-Cys)-NH₂;
SEQ ID NO: 71
Ac-Nle-c(Cys-D-Ala-His-D-Phe-Arg-1-Nal-Cys)-NH₂;
SEQ ID NO: 72
Ac-Nle-c(Cys-D-Ala-His-D-Phe-Arg-Bal-Cys)-NH₂;
SEQ ID NO: 73
Ac-Nle-c(Cys-D-Glu-His-D-Phe-Arg-Trp-Cys)-NH₂;
SEQ ID NO: 74
Ac-Nle-c(Asp-His-D-Phe-Arg-Trp-D-Ala-Lys)-NH₂;
SEQ ID NO: 75
Ac-Nle-c(Cys-D-Ala-His-D-2-Nal-Arg-Bal-Cys)-NH₂;
SEQ ID NO: 76
Ac-Nle-c(Pen-D-Ala-His-D-Phe-Arg-Trp-Cys)-NH₂;
SEQ ID NO: 77
Ac-Nle-c(Cys-D-Ala-His-D-Phe-Arg-Trp-Pen)-NH₂;
SEQ ID NO: 78
Ac-Nle-c(Pen-D-Ala-His-D-Phe-Arg-Trp-Pen)-NH₂;
SEQ ID NO: 79
D-Phe-c(Cys-His-D-Phe-hArg-Trp-β-Ala-D-Cys)-Thr-NH₂;
SEQ ID NO: 80
D-Phe-c(Cys-His-D-(Et)Tyr-Arg-Trp-β-Ala-D-Cys)-Thr-NH₂;
SEQ ID NO: 81
D-Phe-c(Cys-His-D-Phe-Arg-Bip-β-Ala-D-Cys)-Thr-NH₂;
SEQ ID NO: 82
D-Phe-c(Cys-His-D-(Et)Tyr-hArg-Trp-β-Ala-D-Cys)-Thr-NH₂;
SEQ ID NO: 83
D-Phe-c(Cys-His-D-Phe-hArg-Bip-β-Ala-D-Cys)-Thr-NH₂;
SEQ ID NO: 84
D-Phe-c(Cys-His-D-(Et)Tyr-hArg-Bip-β-Ala-D-Cys)-Thr-NH₂;
SEQ ID NO: 85
Nle-c(Cys-His-D-Phe-Arg-Trp-Apn-Cys)-NH₂;
SEQ ID NO: 86
Ac-Nle-c(Asp-D-Ala-His-D-Phe-Arg-Trp-Lys)-NH₂;
SEQ ID NO: 87
Ac-Nle-c(Asp-D-Ala-His-D-Phe-Arg-Bal-Lys)-NH₂;
SEQ ID NO: 88
Ac-Nle-c(Cys-D-Ala-His-D-Phe-Arg-Trp-Pen)-OH;
SEQ ID NO: 89
Ac-Nle-c(Cys-D-Abu-His-D-Phe-Arg-Trp-Cys)-NH₂;
SEQ ID NO: 90
Ac-Nle-c(Cys-D-Val-His-D-Phe-Arg-Trp-Cys)-NH₂;
SEQ ID NO: 91
Ac-Nle-c(Cys-D-Ile-His-D-Phe-Arg-Trp-Cys)-NH₂;
SEQ ID NO: 92
Ac-Nle-c(Cys-D-Leu-His-D-Phe-Arg-Trp-Cys)-NH₂;
SEQ ID NO: 93
Ac-Nle-c(Cys-D-Tle-His-D-Phe-Arg-Trp-Cys)-NH₂;
SEQ ID NO: 94
Ac-Nle-c(Cys-D-Cha-His-D-Phe-Arg-Trp-Cys)-NH₂;
SEQ ID NO: 95
Ac-Nle-c(Pen-His-D-Phe-Arg-Trp-Gaba-Cys)-NH₂;
SEQ ID NO: 96
Ac-Nle-c(Cys-His-D-Phe-Arg-Trp-Gaba-Pen)-NH₂;
SEQ ID NO: 97
Ac-Nle-c(Pen-His-D-Phe-Arg-Trp-Gaba-Pen)-NH₂;
SEQ ID NO: 98
Ac-Leu-c(Cys-His-D-Phe-Arg-Trp-Gaba-Cys)-NH₂;
SEQ ID NO: 99
Ac-Cha-c(Cys-His-D-Phe-Arg-Trp-Gaba-Cys)-NH₂;
SEQ ID NO: 100
Ac-Ile-c(Cys-His-D-Phe-Arg-Trp-Gaba-Cys)-NH₂;
SEQ ID NO: 101
Ac-Phe-c(Cys-His-D-Phe-Arg-Trp-Gaba-Cys)-NH₂;
SEQ ID NO: 102
Ac-Val-c(Cys-His-D-Phe-Arg-Trp-Gaba-Cys)-NH₂;
SEQ ID NO: 103
Ac-2-Nal-c(Cys-His-D-Phe-Arg-Trp-Gaba-Cys)-NH₂;
SEQ ID NO: 104
Nle-c(Cys-His-D-Phe-Arg-Trp-Gaba-Cys)-NH₂;
SEQ ID NO: 105
Phe-c(Cys-His-D-Phe-Arg-Trp-Gaba-Cys)-NH₂;
SEQ ID NO: 106
Ac-Nle-c(Cys-3-Pal-D-Phe-Arg-Trp-Gaba-Cys)-NH₂;
SEQ ID NO: 107
Ac-Nle-c(Cys-D-Ala-His-D-Phe-Arg-Trp-Cys)-OH;
SEQ ID NO: 108
Ac-Nle-c(Cys-His-Phe-Arg-D-Trp-Gaba-Cys)-NH₂;
SEQ ID NO: 109
Ac-Nle-c(Asp-His-D-2-Nal-Arg-Trp-Ala-Lys)-NH₂;
SEQ ID NO: 110
Ac-Nle-c(Asp-His-D-2-Nal-Arg-Trp-β-Ala-Lys)-NH₂;
SEQ ID NO: 111
Ac-Nle-c(Cys-His-D-2-Nal-Arg-Trp-Gaba-Cys)-NH₂;
SEQ ID NO: 112
Ac-Nle-c(Cys-His-D-2-Nal-Arg-Trp-Ahx-Cys)-NH₂;
SEQ ID NO: 113
Ac-hPhe-c(Asp-His-D-2-Nal-Arg-Trp-Gaba-Lys)-NH₂;
SEQ ID NO: 114
Ac-Cha-c(Asp-His-D-2-Nal-Arg-Trp-Gaba-Lys)-NH₂;
SEQ ID NO: 115
Ac-Nle-c(Asp-His-D-Phe-Arg-Trp-β-Ala-Lys)-OH;
SEQ ID NO: 116
Ac-Nle-c(Cys-His-D-Phe-Arg-Trp-Ahx-Cys)-OH;
SEQ ID NO: 117
D-Phe-c(Cys-His-D-Phe-Arg-Trp-Ala-D-Cys)-Thr-OH;
SEQ ID NO: 118
D-Phe-c(Cys-His-D-Phe-Arg-Trp-β-Ala-D-Cys)-Thr-OH;
SEQ ID NO: 119
D-Phe-c(Cys-His-D-Phe-Arg-Trp-Gaba-D-Cys)-Thr-OH;
SEQ ID NO: 120
Ac-Nle-c(Cys-His-D-Phe-Arg-Trp-Apn-Cys)-OH;
SEQ ID NO: 121
Ac-Nle-c(Asp-His-D-Phe-Arg-Trp-Apn-Lys)-OH;
SEQ ID NO: 122
Ac-Cha-c(Asp-His-D-Phe-Arg-Trp-Gaba-Lys)-OH;
SEQ ID NO: 123
Ac-Nle-c(Asp-His-D-Phe-Arg-Trp-Gaba-Lys)-OH;
SEQ ID NO: 124
Ac-Chg-c(Asp-His-D-Phe-Arg-Trp-Gaba-Lys)-OH;
SEQ ID NO: 125
Ac-D-Cha-c(Asp-His-D-Phe-Arg-Trp-Gaba-Lys)-OH;
SEQ ID NO: 126
Ac-hCha-c(Asp-His-D-Phe-Arg-Trp-Gaba-Lys)-OH;
SEQ ID NO: 127
Ac-D-Chg-c(Asp-His-D-Phe-Arg-Trp-Gaba-Lys)-OH;
SEQ ID NO: 128
Ac-hPhe-c(Asp-His-D-Phe-Arg-Trp-Gaba-Lys)-OH;
SEQ ID NO: 129
Ac-Nle-c(Cys-His-D-Phe-Arg-D-Trp-Gaba-Cys)-OH;
SEQ ID NO: 130
Ac-Nle-c(Cys-His-D-Phe-Arg-D-Trp-Ahx-Cys)-OH;
SEQ ID NO: 131
Ac-Nle-c(Cys-His-D-Phe-Arg-D-Trp-β-Ala-Cys)-OH;
SEQ ID NO: 132
Ac-Nle-c(Cys-His-D-Phe-Arg-D-Trp-D-Ala-Cys)-OH;
SEQ ID NO: 133
Ac-Nle-c(Cys-D-Ala-His-D-2-Nal-Arg-Trp-Cys)-OH;
SEQ ID NO: 134
Ac-Nle-c(Cys-D-Ala-His-D-2-Nal-Arg-2-Nal-Cys)-OH;
SEQ ID NO: 135
Ac-Nle-c(Cys-D-Ala-His-D-2-Nal-Arg-1-Nal-Cys)-OH;
SEQ ID NO: 136
Ac-Nle-c(Cys-D-Ala-His-D-2-Nal-Arg-Bal-Cys)-OH;
SEQ ID NO: 137
Ac-Nle-c(Pen-D-Ala-His-D-Phe-Arg-Trp-Cys)-OH;
SEQ ID NO: 138
Ac-Nle-c(Cys-His-D-Phe-Arg-Trp-Gaba-Pen)-OH;
SEQ ID NO: 139
Ac-Arg-c(Cys-D-Ala-His-D-2-Nal-Arg-Trp-Cys)-NH₂;
SEQ ID NO: 140
Ac-Arg-c(Cys-D-Ala-His-D-Phe-Arg-Trp-Cys)-NH₂;
SEQ ID NO: 141
Ac-D-Arg-c(Cys-D-Ala-His-D-Phe-Arg-Trp-Cys)-NH₂;
SEQ ID NO: 142
Ac-D-Arg-c(Cys-D-Ala-His-D-Phe-Arg-Trp-Pen)-NH₂;
SEQ ID NO: 143
Ac-D-Arg-c(Cys-His-D-Phe-Arg-Trp-Gaba-Pen)-NH₂;
SEQ ID NO: 144
Ac-Arg-c(Cys-His-D-Phe-Arg-Trp-Gaba-Pen)-NH₂;
SEQ ID NO: 145
Ac-Arg-c(Cys-D-Ala-His-D-Phe-Arg-Trp-Pen)-NH₂;
SEQ ID NO: 146
Ac-D-Arg-c(Asp-His-D-Phe-Arg-Trp-Ala-Lys)-NH₂;
and
SEQ ID NO: 147
Ac-Arg-c(Asp-His-D-Phe-Arg-Trp-Ala-Lys)-NH₂;

or a pharmaceutically acceptable salt thereof.

In embodiments, the compound of Formula (I) is Ac-Arg-c (Cys-D-Ala-His-D-Phe-Arg-Trp-Cys)-NH₂ (SEQ ID NO: 140) or a pharmaceutically acceptable salt thereof. Ac-Arg-c (Cys-D-Ala-His-D-Phe-Arg-Trp-Cys)-NH₂ (SEQ ID NO: 140), also known as RM-493 and setmelanotide, is a peptide that retains the specificity and functionality of the naturally occurring hormone that activates MC4R and has not been shown to adversely affect blood pressure in clinical trials (see, e.g., Chen et al. J. Clin. Endocrinol. Metab. 2015; 100 (4): 1639-45. The structure of Ac-Arg-c (Cys-D-Ala-His-D-Phe-Arg-Trp-Cys)-NH₂ (SEQ ID NO: 140) is shown below:

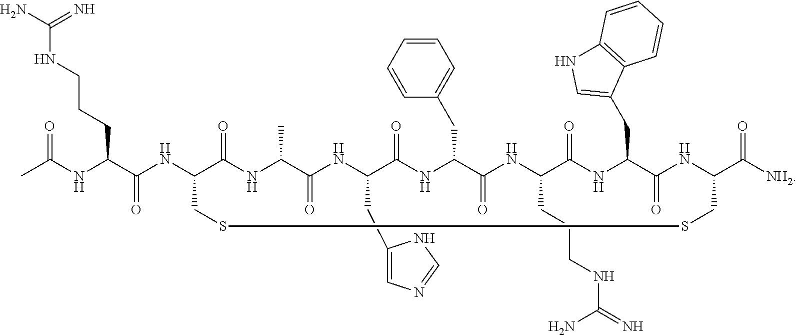 [SMILES: CC(=O)N[C@@H](CCCNC(=N)N)C(=O)N[C@H]1CSSC[C@@H](C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC(=O)[C@@H](C)NC1=O]

In some embodiments, the MC4R agonist is a compound of Formula (II):

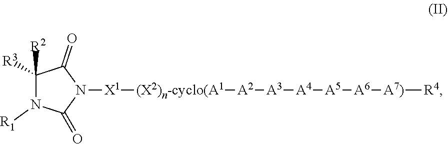

or a pharmaceutically acceptable salt thereof, wherein:
X¹ is

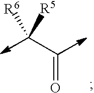

X² is

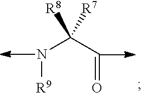

A¹ is Asp, Cys, D-Cys, Dab, Dap, Glu, Lys, Orn, Pen or D-Pen;
A² is an L- or D-amino acid;
A³ is His, 2-Pal, 3-Pal, 4-Pal, (X¹, X², X³, X⁴, X⁵) Phe, Taz, 2-Thi or 3-Thi;
A⁴ is D-Bal, D-1-Nal, D-2-Nal, D-Phe or D-(X¹, X², X³, X⁴, X⁵) Phe;
A⁵ is Arg, hArg, Dab, Dap, Lys or Orn;
A⁶ is Bal, 1-Nal, 2-Nal, (X¹, X², X³, X⁴, X⁵) Phe or Trp;
A⁷ is Asp, Cys, D-Cys, Dab, Dap, Glu, Lys, Orn, Pen or D-Pen;
R¹ is H, (C₁-C₁₀)alkyl or substituted (C₁-C₁₀)alkyl;
R² and R³ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together form a cyclic moiety;
R⁴ is OH, NH₂, CO₂ H or C(O) NH₂;
R⁵ and R⁶ each is, independently, H, (C₁-00)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R⁵ and R⁶ may be fused together form a cyclic moiety;
R⁷ and R⁸ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl; or R⁷ and R⁸ may be fused together form a cyclic moiety;
R⁹ is H, (C₁-C₁₀)alkyl or substituted (C₁-C₁₀)alkyl; and
n is, independently for each occurrence thereof, 0, 1, 2, 3, 4, 5, 6 or 7;
or a pharmaceutically acceptable salt thereof.

In some embodiments of Formula (II), A¹ is Cys; A² is D-Ala, Asn, Asp, Gln, Glu or D-Phe; A³ is H is; A⁴ is D-2-Nal or D-Phe; A⁵ is Arg; A⁶ is Trp; and A⁷ is Cys or Pen; each of R′, R², R³, and R° is, independently, H; R⁴ is C(O) NH₂; each of R⁵ and R⁶ is, independently, H, (C₁-C₁₀) heteroalkyl, substituted (C₁-C₁₀)alkyl or substituted (C₁-C₁₀)heteroalkyl or R⁵ and R⁶ may be fused together form a cyclic moiety; and each of R⁷ and R⁸ is, independently, H, (C₁-C₁₀)alkyl, (C₁-C₁₀)heteroalkyl, substituted (C₁-C₁₀)alkyl or substituted (C₁-C₁₀)heteroalkyl;

or pharmaceutically acceptable salts thereof.

In some embodiments, the compound of Formula (II) is selected from:

(SEQ ID NO: 500)
Hydantoin(C(O)-(Arg-Gly))-c(Cys-Glu-His-D-Phe-Arg-Trp-Cys)-NH₂;
(SEQ ID NO: 501)
Hydantoin(C(O)-(Nle-Gly))-c(Cys-Glu-His-D-Phe-Arg-Trp-Cys)-NH₂;
(SEQ ID NO: 502)
Hydantoin(C(O)-(Gly-Gly))-c(Cys-Glu-His-D-Phe-Arg-Trp-Cys)-NH₂;
(SEQ ID NO: 503)
Hydantoin(C(O)-(Nle-Gly))-c(Cys-D-Ala-His-D-Phe-Arg-Trp-Cys)-NH₂;
(SEQ ID NO: 504)
Hydantoin(C(O)-(Gly-Gly))-c(Cys-D-Ala-His-D-Phe-Arg-Trp-Cys)-NH₂;
(SEQ ID NO: 505)
Hydantoin(C(O)-(Nle-Gly))-c(Cys-D-Ala-His-D-Phe-Arg-Trp-Pen)-NH₂;
(SEQ ID NO: 506)
Hydantoin(C(O)-(Gly-Gly))-c(Cys-D-Ala-His-D-Phe-Arg-Trp-Pen)-NH₂;
(SEQ ID NO: 507)
Hydantoin(C(O)-(Ala-Gly))-c(Cys-D-Ala-His-D-Phe-Arg-Trp-Cys)-NH₂;
(SEQ ID NO: 508)
Hydantoin(C(O)-(D-Ala-Gly))-c(Cys-D-Ala-His-D-Phe-Arg-Trp-Cys)-NH₂;
(SEQ ID NO: 509)
Hydantoin(C(O)-(Aib-Gly))-c(Cys-D-Ala-His-D-Phe-Arg-Trp-Cys)-NH₂;
(SEQ ID NO: 510)
Hydantoin(C(O)-(Val-Gly))-c(Cys-D-Ala-His-D-Phe-Arg-Trp-Cys)-NH₂;
(SEQ ID NO: 511)
Hydantoin(C(O)-(Ile-Gly))-c(Cys-D-Ala-His-D-Phe-Arg-Trp-Cys)-NH₂;
(SEQ ID NO: 512)
Hydantoin(C(O)-(Leu-Gly))-c(Cys-D-Ala-His-D-Phe-Arg-Trp-Cys)-NH₂;
(SEQ ID NO: 513)
Hydantoin(C(O)-(Gly-Gly))-c(Cys-Glu-His-D-2-Nal-Arg-Trp-Cys)-NH₂;
(SEQ ID NO: 514)
Hydantoin(C(O)-(Nle-Gly))-c(Cys-Glu-His-D-2-Nal-Arg-Trp-Cys)-NH₂;
(SEQ ID NO: 515)
Hydantoin(C(O)-(D-Arg-Gly))-c(Cys-Glu-His-D-Phe-Arg-Trp-Cys)-NH₂;
(SEQ ID NO: 516)
Hydantoin(C(O)-(D-Arg-Gly))-c(Cys-D-Ala-His-D-Phe-Arg-Trp-Cys)-NH₂;
(SEQ ID NO: 517)
Hydantoin(C(O)-(Arg-Gly))-c(Cys-D-Ala-His-D-Phe-Arg-Trp-Cys)-NH₂;
(SEQ ID NO: 518)
Hydantoin(C(O)-(D-Arg-Gly))-c(Cys-D-Ala-His-D-2-Nal-Arg-Trp-Cys)-NH₂;
(SEQ ID NO: 519)
Hydantoin(C(O)-(Arg-Gly))-c(Cys-D-Ala-His-D-2-Nal-Arg-Trp-Cys)-NH₂;
(SEQ ID NO: 520)
Hydantoin(C(O)-(Ala-Nle))-c(Cys-Glu-His-D-Phe-Arg-Trp-Cys)-NH₂;
(SEQ ID NO: 521)
Hydantoin(C(O)-(Val-Nle))-c(Cys-Glu-His-D-Phe-Arg-Trp-Cys)-NH₂;
(SEQ ID NO: 522)
Hydantoin(C(O)-(Gly-Nle))-c(Cys-Glu-His-D-Phe-Arg-Trp-Cys)-NH₂;
(SEQ ID NO: 523)
Hydantoin(C(O)-(A6c-Nle))-c(Cys-D-Ala-His-D-Phe-Arg-Trp-Cys)-NH₂;
(SEQ ID NO: 524)
Hydantoin(C(O)-(Gly-Nle))-c(Cys-D-Ala-His-D-Phe-Arg-Trp-Cys)-NH₂;
(SEQ ID NO: 525)
Hydantoin(C(O)-(Ala-Nle))-c(Cys-D-Ala-His-D-Phe-Arg-Trp-Cys)-NH₂;
(SEQ ID NO: 526)
Hydantoin(C(O)-(D-Ala-Nle))-c(Cys-D-Ala-His-D-Phe-Arg-Trp-Cys)-NH₂;
(SEQ ID NO: 527)
Hydantoin(C(O)-(Val-Nle))-c(Cys-D-Ala-His-D-Phe-Arg-Trp-Cys)-NH₂;
(SEQ ID NO: 528)
Hydantoin(C(O)-(Leu-Nle))-c(Cys-D-Ala-His-D-Phe-Arg-Trp-Cys)-NH₂;
(SEQ ID NO: 529)
Hydantoin(C(O)-(Cha-Nle))-c(Cys-D-Ala-His-D-Phe-Arg-Trp-Cys)-NH₂;
(SEQ ID NO: 530)
Hydantoin(C(O)-(Aib-Nle))-c(Cys-D-Ala-His-D-Phe-Arg-Trp-Cys)-NH₂;
(SEQ ID NO: 531)
Hydantoin(C(O)-(Gly-Arg))-c(Cys-Glu-His-D-Phe-Arg-Trp-Cys)-NH₂;
(SEQ ID NO: 532)
Hydantoin(C(O)-(Gly-Arg))-c(Cys-Glu-His-D-2-Nal-Arg-Trp-Cys)-NH₂;
(SEQ ID NO: 533)
Hydantoin(C(O)-(Gly-Arg))-c(Cys-D-Ala-His-D-Phe-Arg-Trp-Cys)-NH₂;
(SEQ ID NO: 534)
Hydantoin(C(O)-(Gly-Arg))-c(Cys-D-Ala-His-D-2-Nal-Arg-Trp-Cys)-NH₂;
(SEQ ID NO: 535)
Hydantoin(C(O)-(Gly-D-Arg))-c(Cys-Glu-His-D-Phe-Arg-Trp-Cys)-NH₂;
(SEQ ID NO: 536)
Hydantoin(C(O)-(Gly-D-Arg))-c(Cys-D-Ala-His-D-Phe-Arg-Trp-Cys)-NH₂;
(SEQ ID NO: 537)
Hydantoin(C(O)-(Gly-D-Arg))-c(Cys-D-Ala-His-D-2-Nal-Arg-Trp-Cys)-NH₂;
and
(SEQ ID NO: 538)
Hydantoin(C(O)-(Nle-Ala))-c(Cys-Glu-His-D-Phe-Arg-Trp-Cys)-NH₂;

or a pharmaceutically acceptable salt thereof.

In some embodiments, the compound of Formula (II) is described in WO2008/147556 or International Patent Application Number PCT/US08/06675, each of which is incorporated herein by reference in its entirety.

In embodiments, the compound of Formula (II) is hydantoin (C(O)-(Arg-Gly))-c (Cys-Glu-His-D-Phe-Arg-Trp-Cys)-NH₂ (SEQ ID NO: 500) or a pharmaceutically acceptable salt thereof, also known as RM-511. The structure of hydantoin (C(O)-(Arg-Gly))-c (Cys-Glu-His-D-Phe-Arg-Trp-Cys)-NH₂ (SEQ ID NO: 500) is shown below:

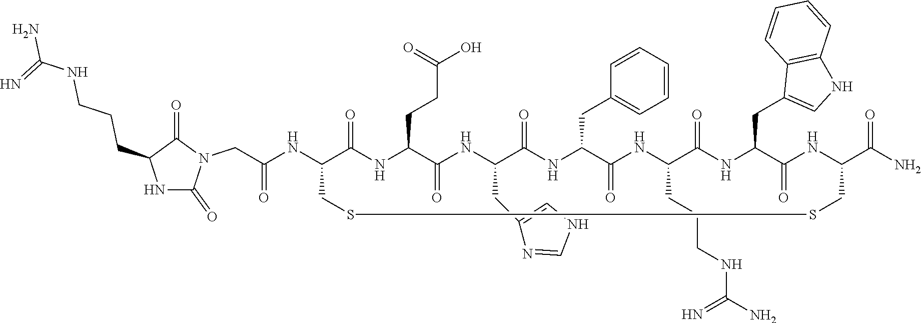 [SMILES: N=C(N)NCCC[C@@H]1NC(=O)[C@@H](Cc2ccccc2)NC(=O)[C@H](Cc2c[nH]cn2)NC(=O)[C@H](CCC(=O)O)NC(=O)[C@@H](NC(=O)CN2C(=O)N[C@@H](CCCNC(=N)N)C2=O)CSSC[C@@H](C(N)=O)NC(=O)[C@H](Cc2c[nH]c3ccccc23)NC1=O]

In some embodiments, the MC4R agonist is a compound of Formula (III):

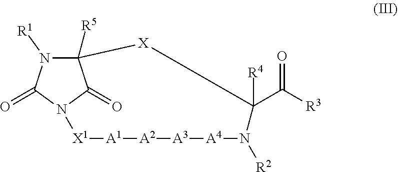

or a pharmaceutically acceptable salt thereof, wherein:
X is selected from the group consisting of —CH₂—S—S—CH₂—, —C(CH₃)₂—S—S—CH₂—, —CH₂—S—S—C(CH₃)₂—, —C(CH₃)₂—S—S—C(CH₃)₂—, —(CH₂)₂—S—S—CH₂—, —CH₂—S—S—(CH₂)₂—, —(CH₂)₂—S—S—(CH₂)₂—, —C(CH₃)₂—S—S—(CH₂)₂—, —(CH₂)₂—S—S—C(CH₃)₂—, —(CH₂)ᵣ—C(O)—NR⁸ (CH₂)ᵣ— and —(CH₂)ᵣ—NR⁸—C(O)—(CH₂)ₜ—;
R² each is, independently, H, (C₁-C₁₀)alkyl or substituted (C₁-C₁₀)alkyl;
R³ is-OH or —NH₂;
R⁴ and R⁵ each is, independently, H, (C₁-C₁₀)alkyl or substituted (C₁-C₁₀)alkyl;
X¹ is

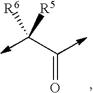

A¹ is His, 2-Pal, 3-Pal, 4-Pal, (X¹, X², X³, X⁴, X⁵) Phe, Taz, 2-Thi, 3-Thi or is deleted;
A² is D-Bal, D-1-Nal, D-2-Nal, D-Phe or D-(X¹, X², X³, X⁴, X⁵) Phe;
A³ is Arg, hArg, Dab, Dap, Lys or Orn;
A⁴ is Bal, 1-Nal, 2-Nal, (X¹, X², X³, X⁴, X⁵) Phe or Trp;
R⁶ and R⁷ each is, independently for each occurrence thereof, H, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl provided that R⁶ and R⁷ may be joined together to form a ring;
R⁸ is H, (C₁-C₁₀)alkyl or substituted (C₁-C₁₀)alkyl;
r is, independently for each occurrence thereof, 1, 2, 3, 4 or 5; and
t is, independently for each occurrence thereof, 1 or 2.

Compounds according the foregoing formula can include compounds wherein X 1 is selected from the group consisting of:

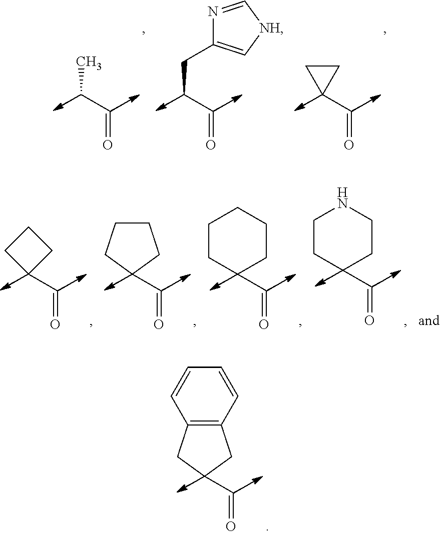

Compounds of Formula (III) are disclosed in International Patent Publication WO 2008/147556 or International Patent Application Number PCT/US08/06675, each of which is incorporated herein by reference in its entirety.

In some embodiments, the compound of Formula (III) is selected from:

(SEQ ID NO: 474)
c[Hydantoin(C(O)-(Cys-D-Ala))-His-D-Phe-Arg-Trp-Cys]-NH₂;
(SEQ ID NO: 475)
c[Hydantoin(C(O)-(hCys-D-Ala))-His-D-Phe-Arg-Trp-Cys]-NH₂;
(SEQ ID NO: 476)
c[Hydantoin(C(O)-(Cys-D-Ala))-His-D-2-Nal-Arg-Trp-Cys]-NH₂;
(SEQ ID NO: 477)
c[Hydantoin(C(O)-(hCys-D-Ala))-His-D-2-Nal-Arg-Trp-Cys]-NH₂;
(SEQ ID NO: 478)
c[Hydantoin(C(O)-(Asp-D-Ala))-His-D-Phe-Arg-Trp-Lys]-NH₂;
(SEQ ID NO: 479)
c[Hydantoin(C(O)-(Asp-D-Ala))-His-D-Phe-Arg-Trp-Orn]-NH₂;
(SEQ ID NO: 480)
c[Hydantoin(C(O)-(Asp-D-Ala))-His-D-Phe-Arg-Trp-Dab]-NH₂;
(SEQ ID NO: 481)
c[Hydantoin(C(O)-(Asp-D-Ala))-His-D-Phe-Arg-Trp-Dap]-NH₂;
(SEQ ID NO: 482)
c[Hydantoin(C(O)-(Asp-His))-D-2-Nal-Arg-Trp-Lys]-NH₂;
(SEQ ID NO: 483)
c[Hydantoin(C(O)-(Asp-His))-D-Phe-Arg-Trp-Lys]-NH₂;
(SEQ ID NO: 484)
c[Hydantoin(C(O)-(Asp-A3c))-D-Phe-Arg-Trp-Lys]-NH₂;
(SEQ ID NO: 485)
c[Hydantoin(C(O)-(Asp-A5c))-D-Phe-Arg-Trp-Lys]-NH₂;
(SEQ ID NO: 486)
c[Hydantoin(C(O)-(Asp-A6c))-D-Phe-Arg-Trp-Lys]-NH₂;
(SEQ ID NO: 487)
c[Hydantoin(C(O)-(Asp-A3c))-D-2-Nal-Arg-Trp-Lys]-NH₂;
(SEQ ID NO: 488)
c[Hydantoin(C(O)-(Asp-A5c))-D-2-Nal-Arg-Trp-Lys]-NH₂;
(SEQ ID NO: 489)
c[Hydantoin(C(O)-(Asp-A6c))-D-2-Nal-Arg-Trp-Lys]-NH₂;
(SEQ ID NO: 490)
c[Hydantoin(C(O)-(Asp-Aic))-D-Phe-Arg-Trp-Lys]-NH₂;
(SEQ ID NO: 491)
c[Hydantoin(C(O)-(Asp-Apc))-D-Phe-Arg-Trp-Lys]-NH₂;
(SEQ ID NO: 492)
c[Hydantoin(C(O)-(Asp-Aic))-D-2-Nal-Arg-Trp-Lys]-NH₂;
(SEQ ID NO: 493)
c[Hydantoin(C(O)-(Asp-Apc))-D-2-Nal-Arg-Trp-Lys]-NH₂;
(SEQ ID NO: 494)
c[Hydantoin(C(O)-(Glu-D-Ala))-His-D-Phe-Arg-Trp-Orn]-NH₂;
(SEQ ID NO: 495)
c[Hydantoin(C(O)-(Glu-D-Ala))-His-D-Phe-Arg-Trp-Dab]-NH₂;
(SEQ ID NO: 496)
c[Hydantoin(C(O)-(Glu-D-Ala))-His-D-Phe-Arg-Trp-Dap]-NH₂;
(SEQ ID NO: 497)
c[Hydantoin(C(O)-(Glu-D-Ala))-His-D-Phe-Arg-Trp-Lys]-NH₂;
(SEQ ID NO: 498)
c[Hydantoin(C(O)-(Glu-His))-D-Phe-Arg-Trp-Dap]-NH₂;
and
(SEQ ID NO: 499)
c[Hydantoin(C(O)-(Glu-His))-D-Phe-Arg-Trp-Lys]-NH₂;
or a pharmaceutically acceptable salt thereof.

In some embodiments, the MC4R agonist is a compound of Formula (IV):

(R²R³)-A¹-c(A²-A³-A⁴-A⁵-A⁶-A⁷-A⁸-A⁹)—NH₂ (IV)

or a pharmaceutically acceptable salt thereof, wherein:
A¹ is Nle or deleted;
   A² is Cys or Asp;
   A³ is Glu or D-Ala;
   A⁴ is His;
   A⁵ is D-Phe;
   A⁶ is Arg;
   A⁷ is Trp, 2-Nal or Bal;
   A⁸ is Gly, Ala, D-Ala, (3-Ala, Gaba or Apn;
   A⁹ is Cys or Lys;
   each of R² and R³ is independently selected from the group consisting of H or (C₁-C₆)acyl.

In exemplary embodiments of Formula (IV):
(I) when R² is (C₁-C₆)acyl, then R³ is H; and
(II) when A² is Cys, then A⁹ is Cys.

Exemplary MC4R agonists of Formula (IV) are disclosed in International Patent Application Publication Number WO 2007/008704, which is incorporated herein by reference in its entirety.

In some embodiments, the compound of Formula (IV) is selected from:

SEQ ID NO: 148
Ac-Nle-c(Cys-D-Ala-His-D-Phe-Arg-Trp-Gly-Cys)-
NH₂;
SEQ ID NO: 149
Ac-Nle-c(Cys-D-Ala-His-D-Phe-Arg-Trp-D-Ala-Cys)-
NH₂;
SEQ ID NO: 150
Ac-Nle-c(Cys-D-Ala-His-D-Phe-Arg-Trp-β-Ala-Cys)-
NH₂;
SEQ ID NO: 151
Ac-Nle-c(Cys-D-Ala-His-D-Phe-Arg-Trp-Gaba-Cys)-
NH₂;
SEQ ID NO: 152
Ac-Nle-c(Cys-D-Ala-His-D-Phe-Arg-Trp-Apn-Cys)-
NH₂;
SEQ ID NO: 153
Ac-c(Cys-Glu-His-D-Phe-Arg-Trp-Ala-Cys)- NH₂;
SEQ ID NO: 154
Ac-c(Cys-Glu-His-D-Phe-Arg-2-Nal-Ala-Cys)-NH₂;
SEQ ID NO: 155
Ac-c(Cys-D-Ala-His-D-Phe-Arg-Trp-Ala-Cys)- NH₂;
SEQ ID NO: 156
Ac-c(Cys-D-Ala-His-D-Phe-Arg-2-Nal-Ala-Cys)- NH₂;
SEQ ID NO: 157
Ac-Nle-c(Cys-D-Ala-His-D-Phe-Arg-Trp-Ala-Cys)-
NH₂;
or
SEQ ID NO: 158
Ac-Nle-c(Asp-D-Ala-His-D-Phe-Arg-Bal-Ala-Lys)-
NH₂;

or a pharmaceutically acceptable salt thereof.

In some embodiments, the MC4R agonist is a compound of Formula (V):

(R²R³)-B1-A¹-c(A²-A³-A⁴-A⁵-A⁶-A⁷-A⁸-A⁹)-A¹⁰-A¹¹-A¹²-A¹³-B²-B³—R¹ (V)

or a pharmaceutically acceptable salt thereof:

B¹ is a peptide moiety which contains 5, 6, 7, 8, 9, 10, 11, 12, 13, 14, or 15 amino acids, wherein at least 5 amino acids are independently selected from the group consisting of L-Arg, D-Arg, L-hArg and D-hArg, or B1 is optionally deleted;
A¹ is Acc, HN—(CH₂) m-C(O), L- or D-amino acid or deleted;
A² is Cys, D-Cys, hCys, D-hCys, Pen, D-Pen, Asp or Glu;
A³ is Gly, Glu, Ala, β-Ala, Gaba, Aib, D-amino acid or deleted;
A⁴ is His, 2-Pal, 3-Pal, 4-Pal, Taz, 2-Thi, 3-Thi or (X′, X², X³, X⁴, X⁵) Phe;
A⁵ is D-Phe, D-1-Nal, D-2-Nal, D-Trp, D-Bal, D-(X¹, X², X³, X⁴, X⁵) Phe, D-(Et) Tyr, D-Dip, D-Bip or D-Bpa;
A⁶ is Arg, hArg, Dab, Dap, Lys, Orn or HN—CH((CH₂) n-N(R⁴R⁵))—C(O);
A⁷ is Trp, 1-Nal, 2-Nal, Bal, Bip, Dip, Bpa, D-Trp, D-1-Nal, D-2-Nal, D-Bal, D-Bip, D-Dip or D-Bpa;
A⁸ is Gly, D-Ala, Acc, Ala, β-Ala, Gaba, Apn, Ahx, Aha, HN—(CH₂)ₛ—C(O) or deleted;
A⁹ is Cys, D-Cys, hCys, D-hCys, Pen, D-Pen, Dab, Dap, Orn or Lys;
A¹⁰ is Acc, HN—(CH₂):—C(O), Pro, hPro, 3-Hyp, 4-Hyp, Thr, an L- or D-amino acid or deleted;
A¹¹ is Pro, hPro, 3-Hyp, 4-Hyp or deleted;
A¹² is Lys, Dab, Dap, Arg, hArg or deleted;
A¹³ is Asp, Glu or deleted;
B² is a peptide moiety containing 1, 2, 3, 4, or 5 amino acids or deleted,
B³ is a peptide moiety which contains 5, 6, 7, 8, 9, 10, 11, 12, 13, 14 or 15 amino acids wherein at least 5 amino acids are independently selected from the group consisting of L-Arg, D-Arg, L-hArg and D-hArg, or is deleted;
R¹ is OH or NH₂;
R² and R³ each is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl and substituted aryl(C₁-C₃₀)acyl;
R⁴ and R⁵ each is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl or C(NH)—NH₂;
n is, independently for each occurrence, 1, 2, 3, 4 or 5;
m is, independently for each occurrence, 1, 2, 3, 4, 5, 6 or 7;
s is, independently for each occurrence, 1, 2, 3, 4, 5, 6 or 7;
t is, independently for each occurrence, 1, 2, 3, 4, 5, 6 or 7;
X¹, X², X³, X⁴ and X⁵ each is, independently for each occurrence, H, F, Cl, Br, I, (C₁-10)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂ or CN.

In some embodiments of Formula (V):
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl or C(NH)—NH₂, then R⁵ is H, (C₁-C₄₀)alkyl, (C₁—C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl or substituted aryl(C₁-C₄₀)alkyl;
(II) when R² is (C₁-C₃₀)acyl, aryl(C₁-C₃₀)acyl, substituted (C₁-C₃₀)acyl or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl or substituted aryl(C₁-C₃₀)alkyl;
(III) neither B¹ nor B² contains one or more of the following amino acid sequences: Arg-(Lys)2-(Arg)2-Gln-(Arg)3, Tyr-Ala-Arg-Lys-Ala-(Arg)2-Gln-Ala-(Arg)2, Tyr-Ala-Arg-(Ala)2-(Arg)2-(Ala)2-(Arg)2, Tyr-Ala-(Arg)9, Tyr-(Ala)3-(Arg)7, Tyr-Ala-Arg-Ala-Pro-(Arg)2-Ala-(Arg)3 or Tyr-Ala-Arg-Ala-Pro-(Arg)2-Pro-(Arg)2;
(IV) either B¹ or B² or both must be present in said compound;
(V) when A² is Cys, D-Cys, hCys, D-hCys, Pen or D-Pen, then A⁹ is Cys, D-Cys, hCys, D-hCys, Pen or D-Pen; and
(VI) when A² is Asp or Glu, then A⁹ is Dab, Dap, Orn or Lys.

In some embodiments of Formula (V):
B¹ is Arg-Lys-Gln-Lys-(Arg)₅, Arg-(Lys)₂-Arg-Gln-(Arg)₄, Arg-(Lys)₂-(Arg)₃-Gln-(Arg)₂, Arg-(Lys)₂-(Arg)₄-Gln-Arg, Arg-(Lys)₂-(Arg)₅-Gln, Arg-(Lys)₂-Gln-(Arg)₅, Arg-Gln-(Lys)₂-(Arg)₅, Arg-Gln-(Arg)₇, Arg-Gln-(Arg)₈, (Arg)₂-Gln-(Arg)₆, (Arg)₂-Gln-(Arg)₇, (Arg)₃-Gln-(Arg)₅, (Arg)₃-Gln-(Arg)₆, (Arg)₄-Gln-(Arg)₄, (Arg)₄-Gln-(Arg)₅, (Arg)₅, (Arg)₅-Gln-(Arg)₃, (Arg)₅-Gln-(Arg)₄, (Arg)₆, (Arg)₆-Gln-(Arg)₃, (Arg)₇, (Arg)₇-Gln-(Arg)₂, (Arg)₈, (Arg) s-Gln-Arg, (Arg)₉, (Arg)₉-Gln, (D-Arg)₅, (D-Arg)₆, (D-Arg)₇, (D-Arg)₈, (D-Arg)₉, Gln-Arg-(Lys)₂-(Arg)₅, Gln-(Arg)₈, Gln-(Arg)₉, Tyr-Gly-Arg-(Lys)₂-(Arg)₂-Gln-(Arg)₃, Tyr-Gly-Arg-(Lys)₂-(Arg)₂-Gln-(Arg)₃-Doc; or deleted;
B² is β-Ala, β-Ala-Gly, β-Ala-Tyr, β-Ala-Tyr-Gly, (β-Ala)₂, (β-Ala)₂-Gly, (β-Ala)₂-Tyr, (β-Ala)₂-Tyr-Gly, Doc, Doc-Gly, Doc-Tyr, Doc-Tyr-Gly, (Doc)₂, (Doc)₂-Gly, (Doc)₂-Tyr, Doc)₂-Tyr-Gly, or deleted;
B³ is Arg-Lys-Gln-Lys-(Arg)₅, Arg-Lys-(Arg)₃-Gln-(Arg)₃, Arg-(Lys)₂-Arg-Gln-(Arg)₄, Arg-(Lys)₂-Gln-(Arg)₅, Arg-(Lys)₂-(Arg)₂-Gln-(Arg)₃, Arg-(Lys)₂-(Arg)₃-Gln-(Arg)₂, Arg-(Lys)₂-(Arg)₄-Gln-Arg, Arg-(Lys)₂-(Arg)₅-Gln, Arg-Gln-(Lys)₂-(Arg)₅, Arg-Gln-(Arg)₇, Arg-Gln-(Arg) s, (Arg)₂-Lys-(Arg)₂-Gln-(Arg)₃, (Arg)₂-Gln-(Arg)₆, (Arg)₂-Gln-(Arg)₇, (Arg)₃-Gln-(Arg)₅, (Arg)₃-Gln-(Arg)₆, (Arg)₄-Gln-(Arg)₄, (Arg)₄-Gln-(Arg)₅, (Arg)₅, (Arg) s-Gln-(Arg)₃, (Arg)₅-Gln-(Arg)₄, (Arg)₆, (Arg)₆-Gln-(Arg)₃, (Arg)₇, (Arg)₇-Gln-(Arg)₂, (Arg)₈, (Arg) s-Gln-Arg, (Arg)₉, (Arg)₉-Gln, (D-Arg)₅, (D-Arg)₆, (D-Arg)₇, (D-Arg)₈, (D-Arg)₉, Gln-Arg-(Lys)₂-(Arg)₅, Gln-(Arg)₈, Gln-(Arg)₉, or deleted;
A¹ is A⁶c, Cha, hCha, Chg, D-Chg, hChg, Gaba, hLeu, Met, β-hMet, D-2-Nal, Nip, Nle, Oic, Phe, D-Phe, hPhe, hPro, or deleted;
A² is Cys;
A³ is D-Abu, Aib, Ala, β-Ala, D-Ala, D-Cha, Gaba, Glu, Gly, D-Ile, D-Leu, D-Met, D-Nle, D-Phe, D-Tle, D-Trp, D-Tyr, D-Val, or deleted;
A⁴ is H;
A⁵ is D-Bal, D-1-Nal, D-2-Nal, D-Phe, D-(X¹, X², X³, X⁴, X⁵) Phe, D-Trp, or D-(Et) Tyr;
A⁶ is Arg or hArg;
A⁷ is Bal, Bip, 1-Nal, 2-Nal, Trp, or D-Trp;
A⁸ is A⁵c, A⁶c, Aha, Ahx, Ala, β-Ala, Apn, Gaba, Gly, or deleted;
A⁹ is Cys, D-Cys, hCys, D-hCys, Lys, Pen, or D-Pen;
A¹⁰ is Pro, Thr or deleted;
A¹¹ is Pro or deleted;
A¹² is arg, Lys, or deleted;
A¹³ is Asp or deleted;
each of R² and R³ is, independently, H or acyl;
   or pharmaceutically acceptable salts thereof.

In some embodiments, the compound of Formula (V) is selected from:

(SEQ ID NO: 159)
Tyr-Gly-Arg-(Lys)₂-(Arg)₂-Gln-(Arg)₃-Nle-c(Asp-His-D-2-Nal-Arg-Trp-Lys)-NH₂;
(SEQ ID NO: 160)
Tyr-Gly-Arg-(Lys)₂-(Arg)₂-Gln-(Arg)₃-Doc-Nle-c(Asp-His-D-2-Nal-Arg-Trp-Lys)-NH₂;
(SEQ ID NO: 161)
Nle-c(Asp-His-D-2-Nal-Arg-Trp-Lys)-β-Ala-Tyr-Gly-Arg-(Lys)₂-(Arg)₂-Gln-(Arg)₃-
NH₂;
(SEQ ID NO: 162)
Ac-Nle-c(Asp-His-D-2-Nal-Arg-Trp-Lys)-β-Ala-Tyr-Gly-Arg-(Lys)₂-(Arg)₂-Gln-(Arg)₃-
NH₂;
(SEQ ID NO: 163)
Nle-c(Asp-His-D-2-Nal-Arg-Trp-Lys)-(Doc)₂-Tyr-Gly-Arg-(Lys)₂-(Arg)₂-Gln-(Arg)₃-
NH₂;
(SEQ ID NO: 164)
Ac-Nle-c(Asp-His-D-2-Nal-Arg-Trp-Lys)-(Pro)₂-Lys-Asp-Tyr-Gly-Arg-(Lys)₂-(Arg)₂-
Gln-(Arg)₃-NH₂;
(SEQ ID NO: 165)
Ac-c(Cys-Glu-His-D-2-Nal-Arg-Trp-Gly-Cys)-(Pro)₂-Lys-Asp-Tyr-Gly-Arg-(Lys)₂-
(Arg)₂-Gln-(Arg)₃-NH₂;
(SEQ ID NO: 166)
Ac-Nle-c(Asp-His-D-2-Nal-Arg-Trp-Lys)-(β-Ala)₂-Tyr-Gly-Arg-(Lys)₂-(Arg)₂-Gln-
(Arg)₃-NH₂;
(SEQ ID NO: 167)
Ac-Nle-c(Asp-His-D-2-Nal-Arg-Trp-Lys)-(Pro)₂-Lys-Asp-Doc-Tyr-Gly-Arg-(Lys)₂-
(Arg)₂-Gln-(Arg)₃-NH₂;
(SEQ ID NO: 168)
Ac-c(Cys-Glu-His-D-2-Nal-Arg-Trp-Gly-Cys)-(Pro)₂-Lys-Asp-Doc-Tyr-Gly-Arg-
(Lys)₂-(Arg)₂-Gln-(Arg)₃-NH₂;
(SEQ ID NO: 169)
Ac-c(Cys-Glu-His-D-2-Nal-Arg-Trp-Ala-Cys)-(Pro)₂-Lys-Asp-β-Ala-Tyr-Gly-Arg-
(Lys)₂-(Arg)₂-Gln-(Arg)₃-NH₂;
(SEQ ID NO: 170)
Ac-c(Cys-Glu-His-D-2-Nal-Arg-Trp-Ala-Cys)-(Pro)₂-Lys-Asp-Doc-Tyr-Gly-Arg-(Lys)₂-
(Arg)₂-Gln-(Arg)₃-NH₂;
(SEQ ID NO: 171)
Ac-Nle-c(Asp-His-D-2-Nal-Arg-Trp-Lys)-(Doc)₂-Tyr-Gly-Arg-(Lys)₂-(Arg)₂-Gln-
(Arg)₃-NH₂;
(SEQ ID NO: 172)
Ac-c(Cys-Glu-His-D-2-Nal-Arg-2-Nal-Ala-Cys)-(Pro)₂-Lys-Asp-β-Ala-Tyr-Gly-Arg-
(Lys)₂-(Arg)₂-Gln-(Arg)₃-NH₂;
(SEQ ID NO: 173)
Ac-c(Cys-Glu-His-D-2-Nal-Arg-Bal-Ala-Cys)-(Pro)₂-Lys-Asp-β-Ala-(Arg)₅-Gln-(Arg)₃-
NH₂;
(SEQ ID NO: 174)
Ac-c(Cys-Glu-His-D-2-Nal-Arg-Bal-Ala-Cys)-(Pro)₂-Lys-Asp-β-Ala-Gly-(Arg)₅-Gln-
(Arg)₃-NH₂;
(SEQ ID NO: 175)
Ac-c(Cys-Glu-His-D-2-Nal-Arg-Bal-Ala-Cys)-(Pro)₂-Lys-Asp-β-Ala-Tyr-Gly-(Arg)₅-
Gln-(Arg)₃-NH₂;
(SEQ ID NO: 176)
Ac-c(Cys-Glu-His-D-2-Nal-Arg-Trp-Ala-Cys)-(Pro)₂-Lys-Asp-β-Ala-Tyr-Gly-Arg-
(Lys)₂-Arg-Gln-(Arg)₄-NH₂;
(SEQ ID NO: 177)
Ac-c(Cys-Glu-His-D-2-Nal-Arg-Trp-Ala-Cys)-(Pro)₂-Lys-Asp-β-Ala-Tyr-Gly-Arg-
(Lys)₂-Gln-(Arg)₅-NH₂;
(SEQ ID NO: 178)
Ac-c(Cys-Glu-His-D-2-Nal-Arg-Trp-Ala-Cys)-(Pro)₂-Lys-Asp-β-Ala-Tyr-Gly-Arg-Lys-
Gln-Lys-(Arg)₅-NH₂;
(SEQ ID NO: 179)
Ac-c(Cys-Glu-His-D-2-Nal-Arg-Trp-Ala-Cys)-(Pro)₂-Lys-Asp-β-Ala-Tyr-Gly-Arg-
(Lys)₂-(Arg)₄-Gln-Arg-NH₂;
(SEQ ID NO: 180)
Ac-c(Cys-Glu-His-D-2-Nal-Arg-Bal-Ala-Cys)-(Pro)₂-Lys-Asp-β-Ala-Tyr-Aib-Arg-
(Lys)₂-(Arg)₂-Gln-(Arg)₃-NH₂;
(SEQ ID NO: 181)
Ac-c(Cys-Glu-His-D-2-Nal-Arg-1-Nal-Ala-Cys)-(Pro)₂-Arg-Asp-β-Ala-(Arg)₅-Gln-
(Arg)₃-NH₂;
(SEQ ID NO: 182)
Ac-c(Cys-Glu-His-D-2-Nal-Arg-1-Nal-Ala-Cys)-(Pro)₂-Lys-Asp-β-Ala-(Arg)₅-Gln-
(Arg)₃-NH₂;
(SEQ ID NO: 183)
Ac-c(Cys-Glu-His-D-2-Nal-Arg-1-Nal-Ala-Cys)-(Pro)₂-Lys-Asp-β-Ala-(Arg)₆-Gln-
(Arg)₃-NH₂;
(SEQ ID NO: 184)
Ac-c(Cys-Glu-His-D-2-Nal-Arg-2-Nal-Ala-Cys)-(Pro)₂-Arg-Asp-β-Ala-(Arg)₅-Gln-
(Arg)₃-NH₂;
(SEQ ID NO: 185)
Ac-c(Cys-Glu-His-D-2-Nal-Arg-2-Nal-Ala-Cys)-(Pro)₂-Lys-Asp-β-Ala-(Arg)₅-Gln-
(Arg)₃-NH₂;
(SEQ ID NO: 186)
Ac-c(Cys-Glu-His-D-2-Nal-Arg-2-Nal-Ala-Cys)-(Pro)₂-Lys-Asp-β-Ala-(Arg)₆-Gln-
(Arg)₃-NH₂;
(SEQ ID NO: 187)
Ac-c(Cys-Glu-His-D-2-Nal-Arg-Bal-Ala-Cys)-(Pro)₂-Arg-Asp-β-Ala-(Arg)₆-Gln-(Arg)₃-
NH₂;
(SEQ ID NO: 188)
Ac-c(Cys-Glu-His-D-2-Nal-Arg-Bal-Ala-Cys)-(Pro)₂-Arg-Asp-β-Ala-(Arg)₅-Gln-(Arg)₃-
NH₂;
(SEQ ID NO: 189)
Ac-c(Cys-Glu-His-D-2-Nal-Arg-Bal-Ala-Cys)-(Pro)₂-Lys-Asp-β-Ala-(Arg)₆-Gln-(Arg)₃-
NH₂;
(SEQ ID NO: 190)
Ac-c(Cys-Glu-His-D-2-Nal-Arg-Trp-Ala-Cys)-(Pro)₂-Lys-Asp-β-Ala-Tyr-Gly-Arg-
(Lys)₂-(Arg)₃-Gln-(Arg)₂-NH₂;
(SEQ ID NO: 191)
Ac-c(Cys-Glu-His-D-2-Nal-Arg-Trp-Ala-Cys)-(Pro)₂-Lys-Asp-β-Ala-Tyr-Gly-Arg-Gln-
(Lys)₂-(Arg)₅-NH₂;
(SEQ ID NO: 192)
Ac-c(Cys-Glu-His-D-2-Nal-Arg-Trp-Ala-Cys)-(Pro)₂-Lys-Asp-β-Ala-Tyr-Gly-Arg-
(Lys)₂-(Arg)₅-Gln-NH₂;
(SEQ ID NO: 193)
Ac-c(Cys-Glu-His-D-2-Nal-Arg-1-Nal-Ala-Cys)-(Pro)₂-Lys-Asp-β-Ala-Tyr-Gly-Arg-
(Lys)₂-(Arg)₂-Gln-(Arg)₃-NH₂;
(SEQ ID NO: 194)
Ac-c(Cys-Glu-His-D-2-Nal-Arg-Bal-Ala-Cys)-(Pro)₂-Lys-Asp-β-Ala-Tyr-Gly-Arg-
(Lys)₂-(Arg)₂-Gln-(Arg)₃-NH₂;
(SEQ ID NO: 195)
Ac-c(Cys-Glu-His-D-2-Nal-Arg-1-Nal-Ala-Cys)-(Pro)₂-Lys-Asp-β-Ala-(Arg)₂-Lys-
(Arg)₂-Gln-(Arg)₃-NH₂;
(SEQ ID NO: 196)
Ac-c(Cys-Glu-His-D-2-Nal-Arg-1-Nal-Ala-Cys)-(Pro)₂-Lys-Asp-β-Ala-Arg-Lys-(Arg)₃-
Gln-(Arg)₃-NH₂;
(SEQ ID NO: 197)
Ac-c(Cys-Glu-His-D-2-Nal-Arg-2-Nal-Ala-Cys)-(Pro)₂-Lys-Asp-β-Ala-(Arg)₂-Lys-
(Arg)₂-Gln-(Arg)₃-NH₂;
(SEQ ID NO: 198)
Ac-c(Cys-Glu-His-D-2-Nal-Arg-2-Nal-Ala-Cys)-(Pro)₂-Lys-Asp-β-Ala-Tyr-Gly-(Arg)₂-
Lys-(Arg)₂-Gln-(Arg)₃-NH₂;
(SEQ ID NO: 199)
Ac-c(Cys-Glu-His-D-2-Nal-Arg-2-Nal-Ala-Cys)-(Pro)₂-Lys-Asp-β-Ala-Gly-(Arg)₂-Lys-
(Arg)₂-Gln-(Arg)₃-NH₂;
(SEQ ID NO: 200)
Ac-c(Cys-Glu-His-D-2-Nal-Arg-2-Nal-Ala-Cys)-(Pro)₂-Lys-Asp-β-Ala-Gly-Arg-Lys-
(Arg)₃-Gln-(Arg)₃-NH₂;
(SEQ ID NO: 201)
Ac-c(Cys-Glu-His-D-2-Nal-Arg-1-Nal-Ala-Cys)-(Pro)₂-Lys-Asp-β-Ala-Tyr-Gly-(Arg)₂-
Lys-(Arg)₂-Gln-(Arg)₃-NH₂;
(SEQ ID NO: 202)
Ac-c(Cys-Glu-His-D-2-Nal-Arg-1-Nal-Ala-Cys)-(Pro)₂-Lys-Asp-β-Ala-Tyr-Gly-Arg-
Lys-(Arg)₃-Gln-(Arg)₃-NH₂;
(SEQ ID NO: 203)
Ac-c(Cys-Glu-His-D-2-Nal-Arg-1-Nal-Ala-Cys)-(Pro)₂-Lys-Asp-β-Ala-Gly-(Arg)₂-Lys-
(Arg)₂-Gln-(Arg)₃-NH₂;
(SEQ ID NO: 204)
Ac-c(Cys-Glu-His-D-2-Nal-Arg-1-Nal-Ala-Cys)-(Pro)₂-Lys-Asp-β-Ala-Gly-Arg-Lys-
(Arg)₃-Gln-(Arg)₃-NH₂;
(SEQ ID NO: 205)
Ac-c(Cys-Glu-His-D-2-Nal-Arg-2-Nal-Ala-Cys)-(Pro)₂-Lys-Asp-β-Ala-(Arg)₂-Lys-
(Arg)₂-Gln-(Arg)₃-NH₂;
(SEQ ID NO: 206)
Ac-c(Cys-Glu-His-D-2-Nal-Arg-2-Nal-Ala-Cys)-(Pro)₂-Lys-Asp-β-Ala-Arg-Lys-(Arg)₃-
Gln-(Arg)₃-NH₂;
(SEQ ID NO: 207)
Ac-c(Cys-Glu-His-D-2-Nal-Arg-2-Nal-Ala-Cys)-(Pro)₂-Lys-Asp-β-Ala-Tyr-Gly-Arg-
Lys-(Arg)₃-Gln-(Arg)₃-NH₂;
(SEQ ID NO: 208)
Ac-c(Cys-Glu-His-D-2-Nal-Arg-Bal-Ala-Cys)-(Pro)₂-Lys-Asp-β-Ala-(Arg)₂-Lys-(Arg)₂-
Gln-(Arg)₃-NH₂;
(SEQ ID NO: 209)
Ac-c(Cys-Glu-His-D-2-Nal-Arg-Bal-Ala-Cys)-(Pro)₂-Lys-Asp-β-Ala-Arg-Lys-(Arg)₃-
Gln-(Arg)₃-NH₂;
(SEQ ID NO: 210)
Ac-c(Cys-Glu-His-D-2-Nal-Arg-Bal-Ala-Cys)-(Pro)₂-Lys-Asp-β-Ala-Tyr-Gly-(Arg)₂-
Lys-(Arg)₂-Gln-(Arg)₃-NH₂;
(SEQ ID NO: 211)
Ac-c(Cys-Glu-His-D-2-Nal-Arg-Bal-Ala-Cys)-(Pro)₂-Lys-Asp-β-Ala-Tyr-Gly-Arg-Lys-
(Arg)₃-Gln-(Arg)₃-NH₂;
(SEQ ID NO: 212)
Ac-c(Cys-Glu-His-D-2-Nal-Arg-Bal-Ala-Cys)-(Pro)₂-Lys-Asp-β-Ala-Gly-(Arg)₂-Lys-
(Arg)₂-Gln-(Arg)₃-NH₂;
(SEQ ID NO: 213)
Ac-c(Cys-Glu-His-D-2-Nal-Arg-Bal-Ala-Cys)-(Pro)₂-Lys-Asp-β-Ala-Gly-Arg-Lys-
(Arg)₃-Gln-(Arg)₃-NH₂;
(SEQ ID NO: 214)
Ac-c(Cys-Glu-His-D-2-Nal-Arg-Trp-Ala-Cys)-(Pro)₂-Lys-Asp-β-Ala-(Arg)₅-Gln-(Arg)₃-
NH₂;
(SEQ ID NO: 215)
Ac-c(Cys-Glu-His-D-2-Nal-Arg-Trp-Ala-Cys)-(Pro)₂-Arg-Asp-β-Ala-(Arg)₅-Gln-(Arg)₃-
NH₂;
(SEQ ID NO: 216)
Ac-c(Cys-Glu-His-D-2-Nal-Arg-Trp-Ala-Cys)-(Pro)₂-Lys-Asp-β-Ala-Tyr-Gly-(Arg)₅-
Gln-(Arg)₃-NH₂;
(SEQ ID NO: 217)
Ac-c(Cys-Glu-His-D-2-Nal-Arg-Trp-Ala-Cys)-(Pro)₂-Arg-Asp-β-Ala-Tyr-Gly-(Arg)₅-
Gln-(Arg)₃-NH₂;
(SEQ ID NO: 218)
Ac-c(Cys-Glu-His-D-2-Nal-Arg-Trp-Ala-Cys)-(Pro)₂-Lys-Asp-β-Ala-(Arg)₅-Gln-(Arg)₄-
NH₂;
(SEQ ID NO: 219)
Ac-c(Cys-Glu-His-D-2-Nal-Arg-Trp-Ala-Cys)-(Pro)₂-Arg-Asp-β-Ala-(Arg)₅-Gln-(Arg)₄-
NH₂;
(SEQ ID NO: 220)
Ac-c(Cys-Glu-His-D-2-Nal-Arg-Trp-Ala-Cys)-(Pro)₂-Lys-Asp-β-Ala-Tyr-Gly-(Arg)₅-
Gln-(Arg)₄-NH₂;
(SEQ ID NO: 221)
Ac-c(Cys-Glu-His-D-2-Nal-Arg-Trp-Ala-Cys)-(Pro)₂-Arg-Asp-β-Ala-Tyr-Gly-(Arg)₅-
Gln-(Arg)₄-NH₂;
(SEQ ID NO: 222)
Ac-c(Cys-Glu-His-D-2-Nal-Arg-1-Nal-Ala-Cys)-(Pro)₂-Lys-Asp-β-Ala-(Arg)₅-Gln-
(Arg)₄-NH₂;
(SEQ ID NO: 223)
Ac-c(Cys-Glu-His-D-2-Nal-Arg-1-Nal-Ala-Cys)-(Pro)₂-Arg-Asp-β-Ala-(Arg)₅-Gln-
(Arg)₄-NH₂;
(SEQ ID NO: 224)
Ac-c(Cys-Glu-His-D-2-Nal-Arg-1-Nal-Ala-Cys)-(Pro)₂-Arg-Asp-β-Ala-(Arg)₆-Gln-
(Arg)₃-NH₂;
(SEQ ID NO: 225)
Ac-c(Cys-Glu-His-D-2-Nal-Arg-1-Nal-Ala-Cys)-(Pro)₂-Lys-Asp-β-Ala-Tyr-Gly-(Arg)₅-
Gln-(Arg)₃-NH₂;
(SEQ ID NO: 226)
Ac-c(Cys-Glu-His-D-2-Nal-Arg-1-Nal-Ala-Cys)-(Pro)₂-Arg-Asp-β-Ala-Tyr-Gly-(Arg)₅-
Gln-(Arg)₃-NH₂;
(SEQ ID NO: 227)
Ac-c(Cys-Glu-His-D-2-Nal-Arg-1-Nal-Ala-Cys)-(Pro)₂-Lys-Asp-β-Ala-Tyr-Gly-(Arg)₅-
Gln-(Arg)₄-NH₂;
(SEQ ID NO: 228)
Ac-c(Cys-Glu-His-D-2-Nal-Arg-1-Nal-Ala-Cys)-(Pro)₂-Arg-Asp-β-Ala-Tyr-Gly-(Arg)₅-
Gln-(Arg)₄-NH₂;
(SEQ ID NO: 229)
Ac-c(Cys-Glu-His-D-2-Nal-Arg-1-Nal-Ala-Cys)-(Pro)₂-Lys-Asp-β-Ala-Tyr-Gly-(Arg)₆-
Gln-(Arg)₃-NH₂;
(SEQ ID NO: 230)
Ac-c(Cys-Glu-His-D-2-Nal-Arg-1-Nal-Ala-Cys)-(Pro)₂-Arg-Asp-β-Ala-Tyr-Gly-(Arg)₆-
Gln-(Arg)₃-NH₂;
(SEQ ID NO: 231)
Ac-c(Cys-Glu-His-D-2-Nal-Arg-2-Nal-Ala-Cys)-(Pro)₂-Arg-Asp-β-Ala-(Arg)₆-Gln-
(Arg)₃-NH₂;
(SEQ ID NO: 232)
Ac-c(Cys-Glu-His-D-2-Nal-Arg-2-Nal-Ala-Cys)-(Pro)₂-Lys-Asp-β-Ala-(Arg)₅-Gln-
(Arg)₄-NH₂;
(SEQ ID NO: 233)
Ac-c(Cys-Glu-His-D-2-Nal-Arg-2-Nal-Ala-Cys)-(Pro)₂-Arg-Asp-β-Ala-(Arg)₅-Gln-
(Arg)₄-NH₂;
(SEQ ID NO: 234)
Ac-c(Cys-Glu-His-D-2-Nal-Arg-2-Nal-Ala-Cys)-(Pro)₂-Lys-Asp-β-Ala-Tyr-Gly-(Arg)₅-
Gln-(Arg)₃-NH₂;
(SEQ ID NO: 235)
Ac-c(Cys-Glu-His-D-2-Nal-Arg-2-Nal-Ala-Cys)-(Pro)₂-Arg-Asp-β-Ala-Tyr-Gly-(Arg)₅-
Gln-(Arg)₃-NH₂;
(SEQ ID NO: 236)
Ac-c(Cys-Glu-His-D-2-Nal-Arg-2-Nal-Ala-Cys)-(Pro)₂-Lys-Asp-β-Ala-Tyr-Gly-(Arg)₆-
Gln-(Arg)₃-NH₂;
(SEQ ID NO: 237)
Ac-c(Cys-Glu-His-D-2-Nal-Arg-2-Nal-Ala-Cys)-(Pro)₂-Arg-Asp-β-Ala-Tyr-Gly-(Arg)₆-
Gln-(Arg)₃-NH₂;
(SEQ ID NO: 238)
Ac-c(Cys-Glu-His-D-2-Nal-Arg-2-Nal-Ala-Cys)-(Pro)₂-Lys-Asp-β-Ala-Tyr-Gly-(Arg)₅-
Gln-(Arg)₄-NH₂;
(SEQ ID NO: 239)
Ac-c(Cys-Glu-His-D-2-Nal-Arg-2-Nal-Ala-Cys)-(Pro)₂-Arg-Asp-β-Ala-Tyr-Gly-(Arg)₅-
Gln-(Arg)₄-NH₂;
(SEQ ID NO: 240)
Ac-c(Cys-Glu-His-D-2-Nal-Arg-Bal-Ala-Cys)-(Pro)₂-Lys-Asp-β-Ala-(Arg)₅-Gln-(Arg)₄-
NH₂;
(SEQ ID NO: 241)
Ac-c(Cys-Glu-His-D-2-Nal-Arg-Bal-Ala-Cys)-(Pro)₂-Arg-Asp-β-Ala-(Arg)₅-Gln-(Arg)₄-
NH₂;
(SEQ ID NO: 242)
Ac-c(Cys-Glu-His-D-2-Nal-Arg-Bal-Ala-Cys)-(Pro)₂-Arg-Asp-β-Ala-Tyr-Gly-(Arg)₅-
Gln-(Arg)₃-NH₂;
(SEQ ID NO: 243)
Ac-c(Cys-Glu-His-D-2-Nal-Arg-Bal-Ala-Cys)-(Pro)₂-Lys-Asp-β-Ala-Tyr-Gly-(Arg)₅-
Gln-(Arg)₄-NH₂;
(SEQ ID NO: 244)
Ac-c(Cys-Glu-His-D-2-Nal-Arg-Bal-Ala-Cys)-(Pro)₂-Arg-Asp-β-Ala-Tyr-Gly-(Arg)₅-
Gln-(Arg)₄-NH₂;
(SEQ ID NO: 245)
Ac-c(Cys-Glu-His-D-2-Nal-Arg-Bal-Ala-Cys)-(Pro)₂-Lys-Asp-β-Ala-Tyr-Gly-(Arg)₆-
Gln-(Arg)₃-NH₂;
(SEQ ID NO: 246)
Ac-c(Cys-Glu-His-D-2-Nal-Arg-Bal-Ala-Cys)-(Pro)₂-Arg-Asp-β-Ala-Tyr-Gly-(Arg)₆-
Gln-(Arg)₃-NH₂;
(SEQ ID NO: 247)
Ac-Nle-c(Cys-D-Ala-His-D-Phe-Arg-Trp-Cys)-(Doc)₂-Tyr-Gly-Arg-(Lys)₂-(Arg)₂-Gln-
(Arg)₃-NH₂;
(SEQ ID NO: 248)
Ac-Nle-c(Cys-D-Ala-His-D-Phe-Arg-Trp-Cys)-β-Ala-Tyr-Gly-Arg-(Lys)₂-Arg-Gln-
(Arg)₄-NH₂;
(SEQ ID NO: 249)
Ac-Nle-c(Cys-D-Ala-His-D-Phe-Arg-Trp-Cys)-Doc-Tyr-Gly-Arg-(Lys)₂-(Arg)₂-Gln-
(Arg)₃-NH₂;
(SEQ ID NO: 250)
Ac-Nle-c(Cys-D-Ala-His-D-Phe-Arg-Trp-Cys)-β-Ala-(Arg)₅-Gln-(Arg)₃-NH₂;
(SEQ ID NO: 251)
Ac-Nle-c(Cys-D-Ala-His-D-Phe-Arg-Trp-Cys)-β-Ala-Gly-(Arg)₅-Gln-(Arg)₃-NH₂;
(SEQ ID NO: 252)
Ac-Nle-c(Cys-D-Ala-His-D-Phe-Arg-Trp-Cys)-β-Ala-Tyr-Gly-(Arg)₅-Gln-(Arg)₃-NH₂;
(SEQ ID NO: 253)
Ac-Nle-c(Cys-D-Ala-His-D-Phe-Arg-Trp-Cys)-β-Ala-Gly-(Arg)₅-Gln-(Arg)₄-NH₂;
(SEQ ID NO: 254)
Ac-Nle-c(Cys-D-Ala-His-D-Phe-Arg-Trp-Cys)-β-Ala-Tyr-Gly-(Arg)₂-Lys-(Arg)₂-Gln-
(Arg)₃-NH₂;
(SEQ ID NO: 255)
Ac-Nle-c(Cys-D-Ala-His-D-Phe-Arg-Trp-Cys)-β-Ala-Tyr-Gly-Arg-Lys-(Arg)₃-Gln-
(Arg)₃-NH₂;
(SEQ ID NO: 256)
Ac-Nle-c(Cys-D-Ala-His-D-Phe-Arg-Trp-Cys)-β-Ala-Gly-(Arg)₂-Lys-(Arg)₂-Gln-
(Arg)₃-NH₂;
(SEQ ID NO: 257)
Ac-Nle-c(Cys-D-Ala-His-D-Phe-Arg-Trp-Cys)-β-Ala-Gly-Arg-Lys-(Arg)₃-Gln-(Arg)₃-
NH₂;
(SEQ ID NO: 258)
Ac-Nle-c(Cys-D-Ala-His-D-Phe-Arg-Trp-Cys)-β-Ala-(Arg)₂-Lys-(Arg)₂-Gln-(Arg)₃-
NH₂;
(SEQ ID NO: 259)
Ac-Nle-c(Cys-D-Ala-His-D-Phe-Arg-Trp-Cys)-β-Ala-Arg-Lys-(Arg)₃-Gln-(Arg)₃-NH₂;
(SEQ ID NO: 260)
Ac-Nle-c(Cys-D-Ala-His-D-Phe-Arg-Trp-Cys)-(β-Ala)₂-(Arg)₅-Gln-(Arg)₃-NH₂;
(SEQ ID NO: 261)
Ac-Nle-c(Cys-D-Ala-His-D-Phe-Arg-Trp-Cys)-(β-Ala)₂-Gly-(Arg)₅-Gln-(Arg)₃-NH₂;
(SEQ ID NO: 262)
Ac-Nle-c(Cys-D-Ala-His-D-Phe-Arg-Trp-Cys)-(β-Ala)₂-Tyr-Gly-(Arg)₅-Gln-(Arg)₃-
NH₂;
(SEQ ID NO: 263)
Ac-Nle-c(Cys-D-Ala-His-D-Phe-Arg-Trp-Cys)-Doc-(Arg)₅-Gln-(Arg)₃-NH₂;
(SEQ ID NO: 264)
Ac-Nle-c(Cys-D-Ala-His-D-Phe-Arg-Trp-Cys)-Doc-Gly-(Arg)₅-Gln-(Arg)₃-NH₂;
(SEQ ID NO: 265)
Ac-Nle-c(Cys-D-Ala-His-D-Phe-Arg-Trp-Cys)-Doc-Tyr-Gly-(Arg)₅-Gln-(Arg)₃-NH₂;
(SEQ ID NO: 266)
Ac-Nle-c(Cys-D-Ala-His-D-Phe-Arg-Trp-Cys)-(Doc)₂-Arg)₅-Gln-(Arg)₃-NH₂;
(SEQ ID NO: 267)
Ac-Nle-c(Cys-D-Ala-His-D-Phe-Arg-Trp-Cys)-(Doc)₂-Gly-(Arg)₅-Gln-(Arg)₃-NH₂;
(SEQ ID NO: 268)
Ac-Nle-c(Cys-D-Ala-His-D-Phe-Arg-Trp-Cys)-(Doc)₂-Tyr-Gly-(Arg)₅-Gln-(Arg)₃-
NH₂;
(SEQ ID NO: 269)
Ac-Nle-c(Cys-D-Ala-His-D-Phe-Arg-Trp-Cys)-β-Ala-(Arg)₅-Gln-(Arg)₄-NH₂;
(SEQ ID NO: 270)
Ac-Nle-c(Cys-D-Ala-His-D-Phe-Arg-Trp-Cys)-β-Ala-Tyr-Gly-(Arg)₅-Gln-(Arg)₄-NH₂;
(SEQ ID NO: 271)
Ac-Nle-c(Cys-D-Ala-His-D-Phe-Arg-Trp-Cys)-(β-Ala)₂-(Arg)₅-Gln-(Arg)₄-NH₂;
(SEQ ID NO: 272)
Ac-Nle-c(Cys-D-Ala-His-D-Phe-Arg-Trp-Cys)-(β-Ala)₂-Gly-(Arg)₅-Gln-(Arg)₄-NH₂;
(SEQ ID NO: 273)
Ac-Nle-c(Cys-D-Ala-His-D-Phe-Arg-Trp-Cys)-(β-Ala)₂-Tyr-Gly-(Arg)₅-Gln-(Arg)₄-
NH₂;
(SEQ ID NO: 274)
Ac-Nle-c(Cys-D-Ala-His-D-Phe-Arg-Trp-Cys)-Doc-(Arg)₅-Gln-(Arg)₄-NH₂;
(SEQ ID NO: 275)
Ac-Nle-c(Cys-D-Ala-His-D-Phe-Arg-Trp-Cys)-Doc-Gly-(Arg)₅-Gln-(Arg)₄-NH₂;
(SEQ ID NO: 276)
Ac-Nle-c(Cys-D-Ala-His-D-Phe-Arg-Trp-Cys)-Doc-Tyr-Gly-(Arg)₅-Gln-(Arg)₄-NH₂;
(SEQ ID NO: 277)
Ac-Nle-c(Cys-D-Ala-His-D-Phe-Arg-Trp-Cys)-(Doc)₂-(Arg)₅-Gln-(Arg)₄-NH₂;
(SEQ ID NO: 278)
Ac-Nle-c(Cys-D-Ala-His-D-Phe-Arg-Trp-Cys)-(Doc)₂-Gly-(Arg)₅-Gln-(Arg)₄-NH₂;
(SEQ ID NO: 279)
Ac-Nle-c(Cys-D-Ala-His-D-Phe-Arg-Trp-Cys)-(Doc)₂-Tyr-Gly-(Arg)₅-Gln-(Arg)₄-
NH₂;
(SEQ ID NO: 280)
Ac-Nle-c(Cys-His-D-Phe-Arg-Trp-Gaba-Cys)-β-Ala-Tyr-Gly-(Arg)₅-Gln-(Arg)₃-NH₂;
(SEQ ID NO: 281)
Ac-Nle-c(Cys-His-D-Phe-Arg-Trp-Gaba-Cys)-β-Ala-(Arg)₅-Gln-(Arg)₃-NH₂;
(SEQ ID NO: 282)
Ac-Nle-c(Asp-His-D-Phe-Arg-Trp-Ala-Lys)-β-Ala-Tyr-Gly-(Arg)₅-Gln-(Arg)₃-NH₂;
(SEQ ID NO: 283)
Ac-Nle-c(Asp-His-D-Phe-Arg-Trp-Ala-Lys)-β-Ala-(Arg)₅-Gln-(Arg)₃-NH₂;
(SEQ ID NO: 284)
Ac-Nle-c(Asp-His-D-Phe-Arg-Trp-Lys)-β-Ala-Tyr-Gly-(Arg)₅-Gln-(Arg)₃-NH₂;
(SEQ ID NO: 285)
Ac-Nle-c(Asp-His-D-Phe-Arg-Trp-Lys)-β-Ala-Gly-(Arg)₅-Gln-(Arg)₃-NH₂;
(SEQ ID NO: 286)
Ac-Nle-c(Asp-His-D-Phe-Arg-Trp-Lys)-β-Ala-(Arg)₅-Gln-(Arg)₃-NH₂;
(SEQ ID NO: 287)
Ac-Nle-c(Asp-His-D-Phe-Arg-Trp-Lys)-(β-Ala)₂-Tyr-Gly-(Arg)₅-Gln-(Arg)₃-NH₂;
(SEQ ID NO: 288)
Ac-Nle-c(Asp-His-D-Phe-Arg-Trp-Lys)-(β-Ala)₂-Gly-(Arg)₅-Gln-(Arg)₃-NH₂;
(SEQ ID NO: 289)
Ac-Nle-c(Asp-His-D-Phe-Arg-Trp-Lys)-(β-Ala)₂-(Arg)₅-Gln-(Arg)₃-NH₂;
(SEQ ID NO: 290)
Ac-Nle-c(Asp-His-D-Phe-Arg-Trp-Lys)-Doc-Tyr-Gly-(Arg)₅-Gln-(Arg)₃-NH₂;
(SEQ ID NO: 291)
Ac-Nle-c(Asp-His-D-Phe-Arg-Trp-Lys)-Doc-Gly-(Arg)₅-Gln-(Arg)₃-NH₂;
(SEQ ID NO: 292)
Ac-Nle-c(Asp-His-D-Phe-Arg-Trp-Lys)-Doc-(Arg)₅-Gln-(Arg)₃-NH₂;
(SEQ ID NO: 293)
Ac-Nle-c(Asp-His-D-Phe-Arg-Trp-Lys)-(Doc)₂-Tyr-Gly-(Arg)₅-Gln-(Arg)₃-NH₂;
(SEQ ID NO: 294)
Ac-Nle-c(Asp-His-D-Phe-Arg-Trp-Lys)-(Doc)₂-Gly-(Arg)₅-Gln-(Arg)₃-NH₂;
(SEQ ID NO: 295)
Ac-Nle-c(Asp-His-D-Phe-Arg-Trp-Lys)-(Doc)₂-(Arg)₅-Gln-(Arg)₃-NH₂;
(SEQ ID NO: 296)
Ac-Nle-c(Asp-His-D-Phe-Arg-Trp-Lys)-β-Ala-Tyr-Gly-(Arg)₅-Gln-(Arg)₄-NH₂;
(SEQ ID NO: 297)
Ac-Nle-c(Asp-His-D-Phe-Arg-Trp-Lys)-β-Ala-Gly-(Arg)₅-Gln-(Arg)₄-NH₂;
(SEQ ID NO: 298)
Ac-Nle-c(Asp-His-D-Phe-Arg-Trp-Lys)-β-Ala-(Arg)₅-Gln-(Arg)₄-NH₂;
(SEQ ID NO: 299)
Ac-Nle-c(Asp-His-D-Phe-Arg-Trp-Lys)-(β-Ala)₂-Tyr-Gly-(Arg)₅-Gln-(Arg)₄-NH₂;
(SEQ ID NO: 300)
Ac-Nle-c(Asp-His-D-Phe-Arg-Trp-Lys)-(β-Ala)₂-Gly-(Arg)₅-Gln-(Arg)₄-NH₂;
(SEQ ID NO: 301)
Ac-Nle-c(Asp-His-D-Phe-Arg-Trp-Lys)-(β-Ala)₂-(Arg)₅-Gln-(Arg)₄-NH₂;
(SEQ ID NO: 302)
Ac-Nle-c(Asp-His-D-Phe-Arg-Trp-Lys)-Doc-Tyr-Gly-(Arg)₅-Gln-(Arg)₄-NH₂;
(SEQ ID NO: 303)
Ac-Nle-c(Asp-His-D-Phe-Arg-Trp-Lys)-Doc-Gly-(Arg)₅-Gln-(Arg)₄-NH₂;
(SEQ ID NO: 304)
Ac-Nle-c(Asp-His-D-Phe-Arg-Trp-Lys)-Doc-(Arg)₅-Gln-(Arg)₄-NH₂;
(SEQ ID NO: 305)
Ac-Nle-c(Asp-His-D-Phe-Arg-Trp-Lys)-(Doc)₂-Tyr-Gly-(Arg)₅-Gln-(Arg)₄-NH₂;
(SEQ ID NO: 306)
Ac-Nle-c(Asp-His-D-Phe-Arg-Trp-Lys)-(Doc)₂-Gly-(Arg)₅-Gln-(Arg)₄-NH₂;
(SEQ ID NO: 307)
Ac-Nle-c(Asp-His-D-Phe-Arg-Trp-Lys)-(Doc)₂-(Arg)₅-Gln-(Arg)₄-NH₂;
(SEQ ID NO: 308)
Ac-Nle-c(Asp-His-D-Phe-Arg-Trp-β-Ala-Lys)-β-Ala-Tyr-Gly-(Arg)₅-Gln-(Arg)₃-NH₂;
(SEQ ID NO: 309)
Ac-Nle-c(Asp-His-D-Phe-Arg-Trp-β-Ala-Lys)-β-Ala-(Arg)₅-Gln-(Arg)₃-NH₂;
(SEQ ID NO: 310)
Ac-Nle-c(Cys-His-D-Phe-Arg-Trp-Ahx-Cys)-β-Ala-Tyr-Gly-(Arg)₅-Gln-(Arg)₃-NH₂;
(SEQ ID NO: 311)
Ac-Nle-c(Cys-His-D-Phe-Arg-Trp-Ahx-Cys)-β-Ala-(Arg)₅-Gln-(Arg)₃-NH₂;
(SEQ ID NO: 312)
D-Phe-c(Cys-His-D-Phe-Arg-Trp-P-Ala-D-Cys)-Thr-β-Ala-Tyr-Gly-(Arg)₅-Gln-(Arg)₃-
NH₂;
(SEQ ID NO: 313)
D-Phe-c(Cys-His-D-Phe-Arg-Trp-P-Ala-D-Cys)-Thr-β-Ala-(Arg)₅-Gln-(Arg)₃-NH₂;
(SEQ ID NO: 314)
Ac-Nle-c(Cys-His-D-Phe-Arg-Trp-Apn-Cys)-β-Ala-Tyr-Gly-(Arg)₅-Gln-(Arg)₃-NH₂;
(SEQ ID NO: 315)
Ac-Nle-c(Cys-His-D-Phe-Arg-Trp-Apn-Cys)-β-Ala-(Arg)₅-Gln-(Arg)₃-NH₂;
(SEQ ID NO: 316)
Ac-Cha-c(Asp-His-D-Phe-Arg-Trp-Gaba-Lys)-β-Ala-Tyr-Gly-(Arg)₅-Gln-(Arg)₃-NH₂;
(SEQ ID NO: 317)
Ac-Cha-c(Asp-His-D-Phe-Arg-Trp-Gaba-Lys)-β-Ala-(Arg)₅-Gln-(Arg)₃-NH₂;
(SEQ ID NO: 318)
Ac-Nle-c(Asp-His-D-Phe-Arg-Trp-Gaba-Lys)-β-Ala-Tyr-Gly-(Arg)₅-Gln-(Arg)₃-NH₂;
(SEQ ID NO: 319)
Ac-Nle-c(Asp-His-D-Phe-Arg-Trp-Gaba-Lys)-β-Ala-(Arg)₅-Gln-(Arg)₃-NH₂;
(SEQ ID NO: 320)
Ac-Chg-c(Asp-His-D-Phe-Arg-Trp-Gaba-Lys)-β-Ala-Tyr-Gly-(Arg)₅-Gln-(Arg)₃-NH₂;
(SEQ ID NO: 321)
Ac-Chg-c(Asp-His-D-Phe-Arg-Trp-Gaba-Lys)-β-Ala-(Arg)₅-Gln-(Arg)₃-NH₂;
(SEQ ID NO: 322)
Ac-hCha-c(Asp-His-D-Phe-Arg-Trp-Gaba-Lys)-β-Ala-Tyr-Gly-(Arg)₅-Gln-(Arg)₃-NH₂;
(SEQ ID NO: 323)
Ac-hCha-c(Asp-His-D-Phe-Arg-Trp-Gaba-Lys)-β-Ala-(Arg)₅-Gln-(Arg)₃-NH₂;
(SEQ ID NO: 324)
Ac-hCha-c(Asp-His-D-Phe-Arg-Trp-Gaba-Lys)-(β-Ala)₂-Tyr-Gly-(Arg)₅-Gln-(Arg)₃-
NH₂;
(SEQ ID NO: 325)
Ac-hCha-c(Asp-His-D-Phe-Arg-Trp-Gaba-Lys)-(β-Ala)₂-(Arg)₅-Gln-(Arg)₃-NH₂;
(SEQ ID NO: 326)
Ac-hCha-c(Asp-His-D-Phe-Arg-Trp-Gaba-Lys)-Doc-Tyr-Gly-(Arg)₅-Gln-(Arg)₃-NH₂;
(SEQ ID NO: 327)
Ac-hCha-c(Asp-His-D-Phe-Arg-Trp-Gaba-Lys)-Doc-(Arg)₅-Gln-(Arg)₃-NH₂;
(SEQ ID NO: 328)
Ac-hCha-c(Asp-His-D-Phe-Arg-Trp-Gaba-Lys)-(Doc)₂-Tyr-Gly-(Arg)₅-Gln-(Arg)₃-
NH₂;
(SEQ ID NO: 329)
Ac-hCha-c(Asp-His-D-Phe-Arg-Trp-Gaba-Lys)-(Doc)₂-(Arg)₅-Gln-(Arg)₃-NH₂;
(SEQ ID NO: 330)
Ac-hCha-c(Asp-His-D-Phe-Arg-Trp-Gaba-Lys)-β-Ala-Tyr-Gly-(Arg)₅-Gln-(Arg)₄-NH₂;
(SEQ ID NO: 331)
Ac-hCha-c(Asp-His-D-Phe-Arg-Trp-Gaba-Lys)-β-Ala-(Arg)₅-Gln-(Arg)₄-NH₂;
(SEQ ID NO: 332)
Ac-hCha-c(Asp-His-D-Phe-Arg-Trp-Gaba-Lys)-(β-Ala)₂-Tyr-Gly-(Arg)₅-Gln-(Arg)₄-
NH₂;
(SEQ ID NO: 333)
Ac-hCha-c(Asp-His-D-Phe-Arg-Trp-Gaba-Lys)-(β-Ala)₂-(Arg)₅-Gln-(Arg)₄-NH₂;
(SEQ ID NO: 334)
Ac-hCha-c(Asp-His-D-Phe-Arg-Trp-Gaba-Lys)-Doc-Tyr-Gly-(Arg)₅-Gln-(Arg)₄-NH₂;
(SEQ ID NO: 335)
Ac-hCha-c(Asp-His-D-Phe-Arg-Trp-Gaba-Lys)-Doc-(Arg)₅-Gln-(Arg)₄-NH₂;
(SEQ ID NO: 336)
Ac-hCha-c(Asp-His-D-Phe-Arg-Trp-Gaba-Lys)-(Doc)₂-Tyr-Gly-(Arg)₅-Gln-(Arg)₄-
NH₂;
(SEQ ID NO: 337)
Ac-hCha-c(Asp-His-D-Phe-Arg-Trp-Gaba-Lys)-(Doc)₂-(Arg)₅-Gln-(Arg)₄-NH₂;
(SEQ ID NO: 338)
Ac-D-Chg-c(Asp-His-D-Phe-Arg-Trp-Gaba-Lys)-β-Ala-Tyr-Gly-(Arg)₅-Gln-(Arg)₃-
NH₂;
(SEQ ID NO: 339)
Ac-D-Chg-c(Asp-His-D-Phe-Arg-Trp-Gaba-Lys)-β-Ala-(Arg)₅-Gln-(Arg)₃-NH₂;
(SEQ ID NO: 340)
Ac-hPhe-c(Asp-His-D-Phe-Arg-Trp-Gaba-Lys)-β-Ala-Tyr-Gly-(Arg)₅-Gln-(Arg)₃-NH₂;
(SEQ ID NO: 341)
Ac-hPhe-c(Asp-His-D-Phe-Arg-Trp-Gaba-Lys)-β-Ala-(Arg)₅-Gln-(Arg)₃-NH₂;
(SEQ ID NO: 342)
Ac-Nle-c(Cys-His-D-Phe-Arg-D-Trp-Apn-Cys)-β-Ala-Tyr-Gly-(Arg)₅-Gln-(Arg)₃-NH₂;
(SEQ ID NO: 343)
Ac-Nle-c(Cys-His-D-Phe-Arg-D-Trp-Apn-Cys)-β-Ala-(Arg)₅-Gln-(Arg)₃-NH₂;
(SEQ ID NO: 344)
Ac-Nle-c(Cys-His-D-Phe-Arg-D-Trp-Ahx-Cys)-β-Ala-Tyr-Gly-(Arg)₅-Gln-(Arg)₃-NH₂;
(SEQ ID NO: 345)
Ac-Nle-c(Cys-His-D-Phe-Arg-D-Trp-Ahx-Cys)-β-Ala-(Arg)₅-Gln-(Arg)₃-NH₂;
(SEQ ID NO: 346)
Ac-Nle-c(Cys-His-D-Phe-Arg-D-Trp-3-Ala-Cys)-β-Ala-Tyr-Gly-(Arg)₅-Gln-(Arg)₃-
NH₂;
(SEQ ID NO: 347)
Ac-Nle-c(Cys-His-D-Phe-Arg-D-Trp-β-Ala-Cys)-β-Ala-(Arg)₅-Gln-(Arg)₃-NH₂;
(SEQ ID NO: 348)
Ac-Nle-c(Cys-D-Ala-His-D-Phe-Arg-Trp-Pen)-β-Ala-Tyr-Gly-(Arg)₅-Gln-(Arg)₃-NH₂;
(SEQ ID NO: 349)
Ac-Nle-c(Cys-D-Ala-His-D-Phe-Arg-Trp-Pen)-β-Ala-Gly-(Arg)₅-Gln-(Arg)₃-NH₂;
(SEQ ID NO: 350)
Ac-Nle-c(Cys-D-Ala-His-D-Phe-Arg-Trp-Pen)-β-Ala-(Arg)₅-Gln-(Arg)₃-NH₂;
(SEQ ID NO: 351)
Ac-Nle-c(Cys-D-Ala-His-D-Phe-Arg-Trp-Pen)-(β-Ala)₂-Tyr-Gly-(Arg)₅-Gln-(Arg)₃-
NH₂;
(SEQ ID NO: 352)
Ac-Nle-c(Cys-D-Ala-His-D-Phe-Arg-Trp-Pen)-(β-Ala)₂-Gly-(Arg)₅-Gln-(Arg)₃-NH₂;
(SEQ ID NO: 353)
Ac-Nle-c(Cys-D-Ala-His-D-Phe-Arg-Trp-Pen)-(β-Ala)₂-(Arg)₅-Gln-(Arg)₃-NH₂;
(SEQ ID NO: 354)
Ac-Nle-c(Cys-D-Ala-His-D-Phe-Arg-Trp-Pen)-Doc-Tyr-Gly-(Arg)₅-Gln-(Arg)₃-NH₂;
(SEQ ID NO: 355)
Ac-Nle-c(Cys-D-Ala-His-D-Phe-Arg-Trp-Pen)-Doc-Gly-(Arg)₅-Gln-(Arg)₃-NH₂;
(SEQ ID NO: 356)
Ac-Nle-c(Cys-D-Ala-His-D-Phe-Arg-Trp-Pen)-Doc-(Arg)₅-Gln-(Arg)₃-NH₂;
(SEQ ID NO: 357)
Ac-Nle-c(Cys-D-Ala-His-D-Phe-Arg-Trp-Pen)-(Doc)₂-Tyr-Gly-(Arg)₅-Gln-(Arg)₃-NH₂;
(SEQ ID NO: 358)
Ac-Nle-c(Cys-D-Ala-His-D-Phe-Arg-Trp-Pen)-(Doc)₂-Gly-(Arg)₅-Gln-(Arg)₃-NH₂;
(SEQ ID NO: 359)
Ac-Nle-c(Cys-D-Ala-His-D-Phe-Arg-Trp-Pen)-(Doc)₂-(Arg)₅-Gln-(Arg)₃-NH₂;
(SEQ ID NO: 360)
D-Phe-c(Cys-His-D-(Et)Tyr-Arg-Trp-P-Ala-D-Cys)-β-Ala-Tyr-Gly-(Arg)₅-Gln-(Arg)₃-
NH₂;
(SEQ ID NO: 361)
D-Phe-c(Cys-His-D-(Et)Tyr-Arg-Trp-β-Ala-D-Cys)-β-Ala-(Arg)₅-Gln-(Arg)₃-NH₂;
(SEQ ID NO: 362)
D-Phe-c(Cys-His-D-(Et)Tyr-Arg-Trp-β-Ala-D-Cys)-β-Ala-Gly-(Arg)₅-Gln-(Arg)₃-NH₂;
(SEQ ID NO: 363)
D-Phe-c(Cys-His-D-(Et)Tyr-Arg-Trp-β-Ala-D-Cys)-β-Ala-(Arg)₅-Gln-(Arg)₄-NH₂;
(SEQ ID NO: 364)
D-Phe-c(Cys-His-D-(Et)Tyr-Arg-Trp-β-Ala-D-Cys)-(β-Ala)₂-Tyr-Gly-(Arg)₅-Gln-
(Arg)₃-NH₂;
(SEQ ID NO: 365)
D-Phe-c(Cys-His-D-(Et)Tyr-Arg-Trp-β-Ala-D-Cys)-(β-Ala)₂-(Arg)₅-Gln-(Arg)₃-NH₂;
(SEQ ID NO: 366)
D-Phe-c(Cys-His-D-(Et)Tyr-Arg-Trp-β-Ala-D-Cys)-(β-Ala)₂-Gly-(Arg)₅-Gln-(Arg)₃-
NH₂;
(SEQ ID NO: 367)
D-Phe-c(Cys-His-D-(Et)Tyr-Arg-Trp-β-Ala-D-Cys)-(β-Ala)₂-(Arg)₅-Gln-(Arg)₄-NH₂;
(SEQ ID NO: 368)
D-Phe-c(Cys-His-D-(Et)Tyr-Arg-Trp-P-Ala-D-Cys)-Doc-Tyr-Gly-(Arg)₅-Gln-(Arg)₃-
NH₂;
(SEQ ID NO: 369)
D-Phe-c(Cys-His-D-(Et)Tyr-Arg-Trp-β-Ala-D-Cys)-Doc-(Arg)₅-Gln-(Arg)₃-NH₂;
(SEQ ID NO: 370)
D-Phe-c(Cys-His-D-(Et)Tyr-Arg-Trp-β-Ala-D-Cys)-Doc-Gly-(Arg)₅-Gln-(Arg)₃-NH₂;
(SEQ ID NO: 371)
D-Phe-c(Cys-His-D-(Et)Tyr-Arg-Trp-β-Ala-D-Cys)-Doc-(Arg)₅-Gln-(Arg)₄-NH₂;
(SEQ ID NO: 372)
D-Phe-c(Cys-His-D-(Et)Tyr-Arg-Trp-β-Ala-D-Cys)-(Doc)₂-Tyr-Gly-(Arg)₅-Gln-(Arg)₃-
NH₂;
(SEQ ID NO: 373)
D-Phe-c(Cys-His-D-(Et)Tyr-Arg-Trp-β-Ala-D-Cys)-(Doc)₂-(Arg)₅-Gln-(Arg)₃-NH₂;
(SEQ ID NO: 374)
D-Phe-c(Cys-His-D-(Et)Tyr-Arg-Trp-β-Ala-D-Cys)-(Doc)₂-Gly-(Arg)₅-Gln-(Arg)₃-
NH₂;
(SEQ ID NO: 375)
D-Phe-c(Cys-His-D-(Et)Tyr-Arg-Trp-β-Ala-D-Cys)-(Doc)₂-(Arg)₅-Gln-(Arg)₄-NH₂;
(SEQ ID NO: 376)
D-Phe-c(Cys-His-D-(Et)Tyr-hArg-Trp-β-Ala-D-Cys)-Thr-β-Ala-Tyr-Gly-(Arg)₅-Gln-
(Arg)₃-NH₂;
(SEQ ID NO: 377)
D-Phe-c(Cys-His-D-(Et)Tyr-hArg-Trp-β-Ala-D-Cys)-Thr-β-Ala-(Arg)₅-Gln-(Arg)₃-
NH₂;
(SEQ ID NO: 378)
D-Phe-c(Cys-His-D-(Et)Tyr-hArg-Trp-β-Ala-D-Cys)-Thr-(β-Ala)₂-Tyr-Gly-(Arg)₅-Gln-
(Arg)₃-NH₂;
(SEQ ID NO: 379)
D-Phe-c(Cys-His-D-(Et)Tyr-hArg-Trp-β-Ala-D-Cys)-Thr-(β-Ala)₂-(Arg)₅-Gln-(Arg)₃-
NH₂;
(SEQ ID NO: 380)
D-Phe-c(Cys-His-D-(Et)Tyr-hArg-Trp-P-Ala-D-Cys)-Thr-Doc-Tyr-Gly-(Arg)₅-Gln-
(Arg)₃-NH₂;
(SEQ ID NO: 381)
D-Phe-c(Cys-His-D-(Et)Tyr-hArg-Trp-β-Ala-D-Cys)-Thr-Doc-(Arg)₅-Gln-(Arg)₃-NH₂;
(SEQ ID NO: 382)
D-Phe-c(Cys-His-D-(Et)Tyr-hArg-Trp-P-Ala-D-Cys)-Thr-(Doc)₂-Tyr-Gly-(Arg)₅-Gln-
(Arg)₃-NH₂;
(SEQ ID NO: 383)
D-Phe-c(Cys-His-D-(Et)Tyr-hArg-Trp-β-Ala-D-Cys)-Thr-β-Ala-Tyr-Gly-(Arg)₅-Gln-
(Arg)₄-NH₂;
(SEQ ID NO: 384)
D-Phe-c(Cys-His-D-(Et)Tyr-hArg-Trp-P-Ala-D-Cys)-Thr-P-Ala-(Arg)₅-Gln-(Arg)₄-
NH₂;
(SEQ ID NO: 385)
D-Phe-c(Cys-His-D-(Et)Tyr-hArg-Trp-β-Ala-D-Cys)-Thr-(β-Ala)₂-Tyr-Gly-(Arg)₅-Gln-
(Arg)₄-NH₂;
(SEQ ID NO: 386)
D-Phe-c(Cys-His-D-(Et)Tyr-hArg-Trp-β-Ala-D-Cys)-Thr-(β-Ala)₂-(Arg)₅-Gln-(Arg)₄-
NH₂;
(SEQ ID NO: 387)
D-Phe-c(Cys-His-D-(Et)Tyr-hArg-Trp-P-Ala-D-Cys)-Thr-Doc-Tyr-Gly-(Arg)₅-Gln-
(Arg)₄-NH₂;
(SEQ ID NO: 388)
D-Phe-c(Cys-His-D-(Et)Tyr-hArg-Trp-β-Ala-D-Cys)-Thr-Doc-(Arg)₅-Gln-(Arg)₄-NH₂;
(SEQ ID NO: 389)
D-Phe-c(Cys-His-D-(Et)Tyr-hArg-Trp-P-Ala-D-Cys)-Thr-(Doc)₂-Tyr-Gly-(Arg)₅-Gln-
(Arg)₄-NH₂;
(SEQ ID NO: 390)
D-Phe-c(Cys-His-D-(Et)Tyr-hArg-Trp-β-Ala-D-Cys)-Thr-(Doc)₂-(Arg)₅-Gln-(Arg)₄-
NH₂;
(SEQ ID NO: 391)
D-Phe-c(Cys-His-D-(Et)Tyr-hArg-Bip-β-Ala-D-Cys)-Thr-β-Ala-Tyr-Gly-(Arg)₅-Gln-
(Arg)₃-NH₂;
(SEQ ID NO: 392)
D-Phe-c(Cys-His-D-(Et)Tyr-hArg-Bip-P-Ala-D-Cys)-Thr-P-Ala-Tyr-Gly-(Arg)₅-Gln-
(Arg)₄-NH₂;
(SEQ ID NO: 393)
D-Phe-c(Cys-His-D-(Et)Tyr-hArg-Bip-P-Ala-D-Cys)-Thr-P-Ala-(Arg)₅-Gln-(Arg)₃-
NH₂;
(SEQ ID NO: 394)
D-Phe-c(Cys-His-D-(Et)Tyr-hArg-Bip-β-Ala-D-Cys)-Thr-(β-Ala)₂-Tyr-Gly-(Arg)₅-Gln-
(Arg)₃-NH₂;
(SEQ ID NO: 395)
D-Phe-c(Cys-His-D-(Et)Tyr-hArg-Bip-β-Ala-D-Cys)-Thr-(β-Ala)₂-(Arg)₅-Gln-(Arg)₃-
NH₂;
(SEQ ID NO: 396)
D-Phe-c(Cys-His-D-(Et)Tyr-hArg-Bip-P-Ala-D-Cys)-Thr-Doc-Tyr-Gly-(Arg)₅-Gln-
(Arg)₃-NH₂;
(SEQ ID NO: 397)
D-Phe-c(Cys-His-D-(Et)Tyr-hArg-Bip-P-Ala-D-Cys)-Thr-Doc-Tyr-Gly-(Arg)₅-Gln-
(Arg)₄-NH₂;
(SEQ ID NO: 398)
D-Phe-c(Cys-His-D-(Et)Tyr-hArg-Bip-P-Ala-D-Cys)-Thr-Doc-(Arg)₅-Gln-(Arg)₃-NH₂;
(SEQ ID NO: 399)
D-Phe-c(Cys-His-D-(Et)Tyr-hArg-Bip-P-Ala-D-Cys)-Thr-(Doc)₂-Tyr-Gly-(Arg)₅-Gln-
(Arg)₃-NH₂;
(SEQ ID NO: 400)
D-Phe-c(Cys-His-D-(Et)Tyr-hArg-Bip-β-Ala-D-Cys)-Thr-(Doc)₂-(Arg)₅-Gln-(Arg)₃-
NH₂;
(SEQ ID NO: 401)
Ac-Nle-c(Cys-D-Ala-His-D-Phe-Arg-Trp-Gly-Cys)-β-Ala-Tyr-Gly-(Arg)₅-Gln-(Arg)₃-
NH₂;
(SEQ ID NO: 402)
Ac-Nle-c(Cys-D-Ala-His-D-Phe-Arg-Trp-Gly-Cys)-β-Ala-(Arg)₅-Gln-(Arg)₃-NH₂;
(SEQ ID NO: 403)
Nle-c(Cys-His-D-Phe-Arg-Trp-Apn-Cys)-β-Ala-Tyr-Gly-(Arg)₅-Gln-(Arg)₃-NH₂;
(SEQ ID NO: 404)
Nle-c(Cys-His-D-Phe-Arg-Trp-Apn-Cys)-β-Ala-(Arg)₅-Gln-(Arg)₃-NH₂;
(SEQ ID NO: 405)
Nle-c(Cys-His-D-Phe-Arg-Trp-Apn-Cys)-(β-Ala)₂-Tyr-Gly-(Arg)₅-Gln-(Arg)₃-NH₂;
(SEQ ID NO: 406)
Nle-c(Cys-His-D-Phe-Arg-Trp-Apn-Cys)-(β-Ala)₂-(Arg)₅-Gln-(Arg)₃-NH₂;
(SEQ ID NO: 407)
Nle-c(Cys-His-D-Phe-Arg-Trp-Apn-Cys)-β-Ala-Tyr-Gly-(Arg)₅-Gln-(Arg)₄-NH₂;
(SEQ ID NO: 408)
Nle-c(Cys-His-D-Phe-Arg-Trp-Apn-Cys)-β-Ala-(Arg)₅-Gln-(Arg)₄-NH₂;
(SEQ ID NO: 409)
Nle-c(Cys-His-D-Phe-Arg-Trp-Apn-Cys)-(β-Ala)₂-Tyr-Gly-(Arg)₅-Gln-(Arg)₄-NH₂;
(SEQ ID NO: 410)
Nle-c(Cys-His-D-Phe-Arg-Trp-Apn-Cys)-(β-Ala)₂-(Arg)₅-Gln-(Arg)₄-NH₂;
(SEQ ID NO: 411)
Nle-c(Cys-His-D-Phe-Arg-Trp-Apn-Cys)-Doc-Tyr-Gly-(Arg)₅-Gln-(Arg)₃-NH₂;
(SEQ ID NO: 412)
Nle-c(Cys-His-D-Phe-Arg-Trp-Apn-Cys)-Doc-(Arg)₅-Gln-(Arg)₃-NH₂;
(SEQ ID NO: 413)
Nle-c(Cys-His-D-Phe-Arg-Trp-Apn-Cys)-(Doc)₂-Tyr-Gly-(Arg)₅-Gln-(Arg)₃-NH₂;
(SEQ ID NO: 414)
Nle-c(Cys-His-D-Phe-Arg-Trp-Apn-Cys)-(Doc)₂-(Arg)₅-Gln-(Arg)₃-NH₂;
(SEQ ID NO: 415)
Nle-c(Cys-His-D-Phe-Arg-Trp-Apn-Cys)-Doc-Tyr-Gly-(Arg)₅-Gln-(Arg)₄-NH₂;
(SEQ ID NO: 416)
Nle-c(Cys-His-D-Phe-Arg-Trp-Apn-Cys)-Doc-(Arg)₅-Gln-(Arg)₄-NH₂;
(SEQ ID NO: 417)
Nle-c(Cys-His-D-Phe-Arg-Trp-Apn-Cys)-(Doc)₂-Tyr-Gly-(Arg)₅-Gln-(Arg)₄-NH₂;
(SEQ ID NO: 418)
Nle-c(Cys-His-D-Phe-Arg-Trp-Apn-Cys)-(Doc)₂-(Arg)₅-Gln-(Arg)₄-NH₂;
(SEQ ID NO: 419)
Ac-Nle-c(Cys-D-Leu-His-D-Phe-Arg-Trp-Cys)-β-Ala-Tyr-Gly-(Arg)₅-Gln-(Arg)₃-NH₂;
(SEQ ID NO: 420)
Ac-Nle-c(Cys-D-Leu-His-D-Phe-Arg-Trp-Cys)-β-Ala-(Arg)₅-Gln-(Arg)₃-NH₂;
(SEQ ID NO: 421)
Ac-Nle-c(Cys-D-Leu-His-D-Phe-Arg-Trp-Cys)-(β-Ala)₂-Tyr-Gly-(Arg)₅-Gln-(Arg)₃-
NH₂;
(SEQ ID NO: 422)
Ac-Nle-c(Cys-D-Leu-His-D-Phe-Arg-Trp-Cys)-(β-Ala)₂-(Arg)₅-Gln-(Arg)₃-NH₂;
(SEQ ID NO: 423)
Ac-Nle-c(Cys-D-Leu-His-D-Phe-Arg-Trp-Cys)-Doc-Tyr-Gly-(Arg)₅-Gln-(Arg)₃-NH₂;
(SEQ ID NO: 424)
Ac-Nle-c(Cys-D-Leu-His-D-Phe-Arg-Trp-Cys)-Doc-(Arg)₅-Gln-(Arg)₃-NH₂;
(SEQ ID NO: 425)
Ac-Nle-c(Cys-D-Leu-His-D-Phe-Arg-Trp-Cys)-(Doc)₂-Tyr-Gly-(Arg)₅-Gln-(Arg)₃-
NH₂;
(SEQ ID NO: 426)
Ac-Nle-c(Cys-D-Leu-His-D-Phe-Arg-Trp-Cys)-(Doc)₂-(Arg)₅-Gln-(Arg)₃-NH₂;
(SEQ ID NO: 427)
Ac-Nle-c(Cys-D-Leu-His-D-Phe-Arg-Trp-Cys)-β-Ala-Tyr-Gly-(Arg)₅-Gln-(Arg)₄-NH₂;
(SEQ ID NO: 428)
Ac-Nle-c(Cys-D-Leu-His-D-Phe-Arg-Trp-Cys)-β-Ala-(Arg)₅-Gln-(Arg)₄-NH₂;
(SEQ ID NO: 429)
Ac-Nle-c(Cys-D-Leu-His-D-Phe-Arg-Trp-Cys)-β-Ala)₂-Tyr-Gly-(Arg)₅-Gln-(Arg)₄-
NH₂;
(SEQ ID NO: 430)
Ac-Nle-c(Cys-D-Leu-His-D-Phe-Arg-Trp-Cys)-(β-Ala)₂-(Arg)₅-Gln-(Arg)₄-NH₂;
(SEQ ID NO: 431)
Ac-Nle-c(Cys-D-Leu-His-D-Phe-Arg-Trp-Cys)-Doc-Tyr-Gly-(Arg)₅-Gln-(Arg)₄-NH₂;
(SEQ ID NO: 432)
Ac-Nle-c(Cys-D-Leu-His-D-Phe-Arg-Trp-Cys)-Doc-(Arg)₅-Gln-(Arg)₄-NH₂;
(SEQ ID NO: 433)
Ac-Nle-c(Cys-D-Leu-His-D-Phe-Arg-Trp-Cys)-(Doc)₂-Tyr-Gly-(Arg)₅-Gln-(Arg)₄-
NH₂;
(SEQ ID NO: 434)
Ac-Nle-c(Cys-D-Leu-His-D-Phe-Arg-Trp-Cys)-(Doc)₂-(Arg)₅-Gln-(Arg)₄-NH₂;
(SEQ ID NO: 435)
Ac-Nle-c(Cys-D-Cha-His-D-Phe-Arg-Trp-Cys)-β-Ala-Tyr-Gly-(Arg)₅-Gln-(Arg)₃-NH₂;
(SEQ ID NO: 436)
Ac-Nle-c(Cys-D-Cha-His-D-Phe-Arg-Trp-Cys)-β-Ala-(Arg)₅-Gln-(Arg)₃-NH₂;
(SEQ ID NO: 437)
Ac-Nle-c(Cys-D-Cha-His-D-Phe-Arg-Trp-Cys)-(β-Ala)₂-Tyr-Gly-(Arg)₅-Gln-(Arg)₃-
NH₂;
(SEQ ID NO: 438)
Ac-Nle-c(Cys-D-Cha-His-D-Phe-Arg-Trp-Cys)-(β-Ala)₂-(Arg)₅-Gln-(Arg)₃-NH₂;
(SEQ ID NO: 439)
Ac-Nle-c(Cys-D-Cha-His-D-Phe-Arg-Trp-Cys)-Doc-Tyr-Gly-(Arg)₅-Gln-(Arg)₃-NH₂;
(SEQ ID NO: 440)
Ac-Nle-c(Cys-D-Cha-His-D-Phe-Arg-Trp-Cys)-Doc-(Arg)₅-Gln-(Arg)₃-NH₂;
(SEQ ID NO: 441)
Ac-Nle-c(Cys-D-Cha-His-D-Phe-Arg-Trp-Cys)-(Doc)₂-Tyr-Gly-(Arg)₅-Gln-(Arg)₃-
NH₂;
(SEQ ID NO: 442)
Ac-Nle-c(Cys-D-Cha-His-D-Phe-Arg-Trp-Cys)-(Doc)₂-(Arg)₅-Gln-(Arg)₃-NH₂;
(SEQ ID NO: 443)
Ac-Nle-c(Cys-D-Cha-His-D-Phe-Arg-Trp-Cys)-β-Ala-Tyr-Gly-(Arg)₅-Gln-(Arg)₄-NH₂;
(SEQ ID NO: 444)
Ac-Nle-c(Cys-D-Cha-His-D-Phe-Arg-Trp-Cys)-β-Ala-(Arg)₅-Gln-(Arg)₄-NH₂;
(SEQ ID NO: 445)
Ac-Nle-c(Cys-D-Cha-His-D-Phe-Arg-Trp-Cys)-(β-Ala)₂-Tyr-Gly-(Arg)₅-Gln-(Arg)₄-
NH₂;
(SEQ ID NO: 446)
Ac-Nle-c(Cys-D-Cha-His-D-Phe-Arg-Trp-Cys)-(β-Ala)₂-(Arg)₅-Gln-(Arg)₄-NH₂;
(SEQ ID NO: 447)
Ac-Nle-c(Cys-D-Cha-His-D-Phe-Arg-Trp-Cys)-Doc-Tyr-Gly-(Arg)₅-Gln-(Arg)₄-NH₂;
(SEQ ID NO: 448)
Ac-Nle-c(Cys-D-Cha-His-D-Phe-Arg-Trp-Cys)-Doc-(Arg)₅-Gln-(Arg)₄-NH₂;
(SEQ ID NO: 449)
Ac-Nle-c(Cys-D-Cha-His-D-Phe-Arg-Trp-Cys)-(Doc)₂-Tyr-Gly-(Arg)₅-Gln-(Arg)₄-NH₂;
(SEQ ID NO: 450)
Ac-Nle-c(Cys-D-Cha-His-D-Phe-Arg-Trp-Cys)-(Doc)₂-(Arg)₅-Gln-(Arg)₄-NH₂;
(SEQ ID NO: 451)
Nle-c(Cys-His-D-Phe-Arg-Trp-Gaba-Cys)-β-Ala-Tyr-Gly-(Arg)₅-Gln-(Arg)₃-NH₂;
(SEQ ID NO: 452)
Nle-c(Cys-His-D-Phe-Arg-Trp-Gaba-Cys)-β-Ala-(Arg)₅-Gln-(Arg)₃-NH₂;
(SEQ ID NO: 453)
Nle-c(Cys-His-D-Phe-Arg-Trp-Gaba-Cys)-(β-Ala)₂-Tyr-Gly-(Arg)₅-Gln-(Arg)₃-NH₂;
(SEQ ID NO: 454)
Nle-c(Cys-His-D-Phe-Arg-Trp-Gaba-Cys)-(β-Ala)₂-(Arg)₅-Gln-(Arg)₃-NH₂;
(SEQ ID NO: 455)
Nle-c(Cys-His-D-Phe-Arg-Trp-Gaba-Cys)-β-Ala-Tyr-Gly-(Arg)₅-Gln-(Arg)₄-NH₂;
(SEQ ID NO: 456)
Nle-c(Cys-His-D-Phe-Arg-Trp-Gaba-Cys)-β-Ala-(Arg)₅-Gln-(Arg)₄-NH₂;
(SEQ ID NO: 457)
Nle-c(Cys-His-D-Phe-Arg-Trp-Gaba-Cys)-(β-Ala)₂-Tyr-Gly-(Arg)₅-Gln-(Arg)₄-NH₂;
(SEQ ID NO: 458)
Nle-c(Cys-His-D-Phe-Arg-Trp-Gaba-Cys)-(β-Ala)₂-(Arg)₅-Gln-(Arg)₄-NH₂;
(SEQ ID NO: 459)
Nle-c(Cys-His-D-Phe-Arg-Trp-Gaba-Cys)-Doc-Tyr-Gly-(Arg)₅-Gln-(Arg)₃-NH₂;
(SEQ ID NO: 460)
Nle-c(Cys-His-D-Phe-Arg-Trp-Gaba-Cys)-Doc-(Arg)₅-Gln-(Arg)₃-NH₂;
(SEQ ID NO: 461)
Nle-c(Cys-His-D-Phe-Arg-Trp-Gaba-Cys)-(Doc)₂-Tyr-Gly-(Arg)₅-Gln-(Arg)₃-NH₂;
(SEQ ID NO: 462)
Nle-c(Cys-His-D-Phe-Arg-Trp-Gaba-Cys)-(Doc)₂-(Arg)₅-Gln-(Arg)₃-NH₂;
(SEQ ID NO: 463)
Nle-c(Cys-His-D-Phe-Arg-Trp-Gaba-Cys)-Doc-Tyr-Gly-(Arg)₅-Gln-(Arg)₄-NH₂;
(SEQ ID NO: 464)
Nle-c(Cys-His-D-Phe-Arg-Trp-Gaba-Cys)-Doc-(Arg)₅-Gln-(Arg)₄-NH₂;
(SEQ ID NO: 465)
Nle-c(Cys-His-D-Phe-Arg-Trp-Gaba-Cys)-(Doc)₂-Tyr-Gly-(Arg)₅-Gln-(Arg)₄-NH₂;
or
(SEQ ID NO: 466)
Nle-c(Cys-His-D-Phe-Arg-Trp-Gaba-Cys)-(Doc)₂-(Arg)₅-Gln-(Arg)₄-NH₂;

or pharmaceutically acceptable salts thereof.

In some embodiments, a compound of Formula (V) is disclosed in International Application Publication Number WO 2007/008684, which is incorporated herein by reference in its entirety.

In some embodiments, the MC4R agonist is a compound of Formula (VI):

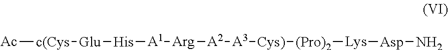

or pharmaceutically acceptable salts thereof. In Formula (IV):
A¹ is the D-isomer of X-Phe or 2-Nal where X is halogen;
A² is Bal, 1-Nal, 2-Nal, or Trp; and
A³ is Aib, Ala, β-Ala or Gly,

In some embodiments, the compound of Formula (VI) is selected from:

(SEQ ID NO: 467)
Ac-c(Cys-Glu-His-D-4-Br-Phe-Arg-Trp-Gly-Cys)-
(Pro)₂-Lys-Asp-NH₂;
(SEQ ID NO: 468)
Ac-c(Cys-Glu-His-D-2-Nal-Arg-Trp-Ala-Cys)-
(Pro)₂-Lys-Asp-NH₂;
(SEQ ID NO: 469)
Ac-c(Cys-Glu-His-D-2-Nal-Arg-2-Nal-Ala-Cys)-
(Pro)₂-Lys-Asp-NH₂;
(SEQ ID NO: 470)
Ac-c(Cys-Glu-His-D-2-Nal-Arg-1-Nal-Ala-Cys)-
(Pro)₂-Lys-Asp-NH₂;
(SEQ ID NO: 471)
Ac-c(Cys-Glu-His-D-2-Nal-Arg-Bal-Ala-Cys)-
(Pro)₂-Lys-Asp-NH₂;
(SEQ ID NO: 472)
Ac-c(Cys-Glu-His-D-2-Nal-Arg-2-Nal-β-Ala-Cys)-
(Pro)₂-Lys-Asp-NH₂;
or
(SEQ ID NO: 473)
Ac-c(Cys-Glu-His-D-2-Nal-Arg-2-Nal-Aib-Cys)-
(Pro)₂-Lys-Asp-NH₂;

or pharmaceutically acceptable salts thereof.

In an example embodiment, the MC4R agonist is a compound of Formula (VII):

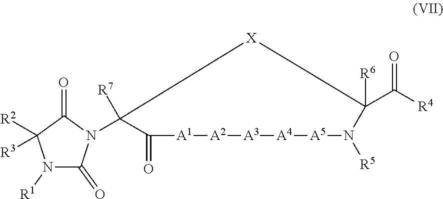

or a pharmaceutically acceptable salt thereof wherein:

X is selected from the group consisting of —CH₂—S—S—CH₂—, —C(CH₃)₂SSCH₂—, —CH₂—S—S—C(CH₃)₂—, —C(CH₃)₂—S—S—C(CH₃) z-, —(CH₂)₂—S—S—CH₂—, —CH₂—S—S—(CH₂)₂, (CH₂)₂—S—S—(CH₂)₂—, —C(CH₃)₂—S—S—(CH₂)₂—, —(CH₂)₂—S—S—C(CH₃)₂—, —(CH₂)ᵣ-C(O)—NR⁸—(CH₂)ᵣ— and —(CH₂), —NR⁸—C(O)—(CH₂)ₜ—;
each of R¹ and R⁵ is, independently, H, (C₁-C₁₀)alkyl or substituted (C₁-C₁₀)alkyl;
each of R² and R³ is, independently, H, (C₁-C₁₀)alkyl, (C₁-00)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together to form a ring;
R⁴ is OH or NH₂;
each of R⁶ and R⁷ is, independently, H, (C₁-C₁₀)alkyl or substituted (C₁-C₁₀)alkyl;
A¹ is an L- or D-amino acid or deleted;
A² is His, 2-Pal, 3-Pal, 4-Pal, (X¹, X², X³, X⁴, X⁵) Phe, Taz, 2-Thi or 3-Thi;
A³ is D-Bal, D-1-Nal, D-2-Nal, D-Phe or D-(X¹, X², X³, X⁴, X⁵) Phe;
A⁴ is Arg, hArg, Dab, Dap, Lys or Orn;
A⁵ is Bal, 1-Nal, 2-Nal, (X¹, X², X³, X⁴, X⁵) Phe or Trp;
r is, independently for each occurrence thereof, 1, 2, 3, 4 or 5; and
t is, independently for each occurrence thereof, 1 or 2;
   or pharmaceutically acceptable salts thereof.

In an example embodiment of the compounds of Formula (VII),
A¹ is Ala, D-Ala, Asn, Asp, Gln, Glu or Gly.

Example compounds according to Formula (VII) include:

(SEQ ID NO: 539)
c[Hydantoin(C(O)-(Nle-Cys))-D-Ala-His-D-Phe-Arg-
Trp-Cys]-NH₂;
(SEQ ID NO: 540)
c[Hydantoin(C(O)-(Ala-Cys))-D-Ala-His-D-Phe-Arg-
Trp-Cys]-NH₂;
(SEQ ID NO: 541)
c[Hydantoin(C(O)-(D-Ala-Cys))-D-Ala-His-D-Phe-
Arg-Trp-Cys]-NH₂;
(SEQ ID NO: 542)
c[Hydantoin(C(O)-(Aib-Cys))-D-Ala-His-D-Phe-Arg-
Trp-Cys]-NH₂;
(SEQ ID NO: 543)
c[Hydantoin(C(O)-(Val-Cys))-D-Ala-His-D-Phe-Arg-
Trp-Cys]-NH₂;
(SEQ ID NO: 544)
c[Hydantoin(C(O)-(Abu-Cys))-D-Ala-His-D-Phe-Arg-
Trp-Cys]-NH₂;
(SEQ ID NO: 545)
c[Hydantoin(C(O)-(Leu-Cys))-D-Ala-His-D-Phe-Arg-
Trp-Cys]-NH₂;
(SEQ ID NO: 546)
c[Hydantoin(C(O)-(Ile-Cys))-D-Ala-His-D-Phe-Arg-
Trp-Cys]-NH2;
(SEQ ID NO: 547)
c[Hydantoin(C(O)-(Cha-Cys))-D-Ala-His-D-Phe-Arg-
Trp-Cys]-NH₂;
(SEQ ID NO: 548)
c[Hydantoin(C(O)-(A6c-Cys))-D-Ala-His-D-Phe-Arg-
Trp-Cys]-NH₂;
(SEQ ID NO: 549)
c[Hydantoin(C(O)-(Phe-Cys))-D-Ala-His-D-Phe-Arg-
Trp-Cys]-NH₂;
(SEQ ID NO: 550)
c[Hydantoin(C(O)-(Gly-Cys))-D-Ala-His-D-Phe-Arg-
Trp-Cys]-NH₂;
or
(SEQ ID NO: 551)
c[Hydantoin(C(O)-(Gly-Cys))-Glu-His-D-Phe-Arg-
Trp-Cys]-NH₂;

or pharmaceutically acceptable salts thereof.

In some embodiments, a compound of Formula (VII) is disclosed in International Application Publication Number WO2008/147556, which is incorporated herein by reference in its entirety.

In some embodiments, the MC4R agonist is a compound of Formula (VIII):

(R²R³)-A⁰-A¹-c(A²-A³-A⁴-A⁵-A⁶-A⁷-A⁸-A⁹)-A¹⁰-R¹ (VIII)

or a pharmaceutically acceptable salt thereof wherein:
A⁰ is an aromatic amino acid
A¹ is Acc, HN—(CH₂)ₘ—C(O), an L- or D-amino acid;
A² is Asp, Cys, D-Cys, hCys, D-hCys, Glu, Pen, or D-Pen;
A³ is Aib, Ala, β-Ala, Gaba, Gly or a D-amino acid;
A⁴ is His, 2-Pal, 3-Pal, 4-Pal, (X¹, X², X³, X⁴, X⁵) Phe, Taz, 2-Thi, or 3-Thi;
A⁵ is D-Bal, D-1-Nal, D-2-Nal, D-Phe, L-Phe, D-(X¹, X², X³, X⁴, X⁵) Phe, L-Phe, D-Trp or D-(Et) Tyr;
A⁶ is Arg, hArg, Dab, Dap, Lys, Orn, or HN—CH((CH₂) n-N(R⁴R⁵))—C(O);
A⁷ is Bal, D-Bal, Bip, D-Bip, 1-Nal, D-1-Nal, 2-Nal, D-2-Nal, or D-Trp;
A⁸ is Acc, Aha, Ahx, Ala, D-Ala, β-Ala, Apn, Gaba, Gly, HN—(CH₂) s-C(O), or deleted;
A⁹ is Cys, D-Cys, hCys, D-hCys, Dab, Dap, Lys, Orn, Pen, or D-Pen;
A¹⁰ is Acc, HN—(CH₂)ᵣ—C(O), L- or D-amino acid, or deleted;
R¹ is OH, or NH₂;
each of R² and R³ is, independently for each occurrence selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁—C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀) allyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X¹, X², X³, X⁴, and X⁵ each is, independently for each occurrence, H, F, C₁, Br, I, (C₁-10)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.

In example embodiments of Formula (VIII),
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or —C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl;
(II) when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;
(III) when A² is Cys, D-Cys, hCys, D-hCys, Pen, or D-Pen, then A⁹ is Cys, D-Cys, hCys, D-hCys, Pen, or D-Pen;
(IV) when A² is Asp or Glu, then A⁹ is Dab, Dap, Orn, or Lys;
(V) when A⁸ is Ala or Gly, then A¹ is not Nle; or pharmaceutically acceptable salts thereof.

In example embodiments of compounds of Formula (VIII):
A⁰ is 1-Nal, 2-Nal, H is, Pff, Phe, Trp, or Tyr; A¹ is Arg; A² is Cys; A³ is D-Ala; A⁴ is H; A⁵ is D-Phe; A⁶ is Arg; A⁷ is Trp; A⁸ is deleted; A⁹ is Cys; and A¹⁰ is deleted; or pharmaceutically acceptable salts thereof.

Particular compounds of the immediately foregoing group of compounds include:

(SEQ ID NO: 552)
Ac-Tyr-Arg-c(Cys-D-Ala-His-D-Phe-Arg-Trp-Cys)-NH₂;
(SEQ ID NO: 553)
Ac-2-Nal-Arg-c(Cys-D-Ala-His-D-Phe-Arg-Trp-Cys)-
NH₂;
(SEQ ID NO: 554)
Ac-1-Nal-Arg-c(Cys-D-Ala-His-DPhe-Arg-Trp-Cys)-NH₂;
(SEQ ID NO: 555)
Ac-Phe-Arg-c(Cys-D-Ala-His-D-Phe-Arg-Trp-Cys)-NH₂;
(SEQ ID NO: 556)
Ac-Trp-Arg-c(Cys-D-Ala-His-D-Phe-Arg-Trp-Cys)-NH₂;
(SEQ ID NO: 557)
Ac-Pff-Arg-c(Cys-D-Ala-His-D-Phe-Arg-Trp-Cys)-NH₂;
(SEQ ID NO: 558)
H-His-Arg-c(Cys-D-Ala-His-D-Phe-Arg-Trp-Cys)-NH₂;
or
(SEQ ID NO: 559)
Ac-His-Arg-c(Cys-D-Ala-His-D-Phe-Arg-Trp-Cys)-NH₂;

or a pharmaceutically acceptable salt thereof.

In some embodiments, the MC4R agonist is an agonist described in WO2014/144260 A1, incorporated herein by reference.

In one example embodiment, an MC4R agonist is a compound represented by structural formula (X):

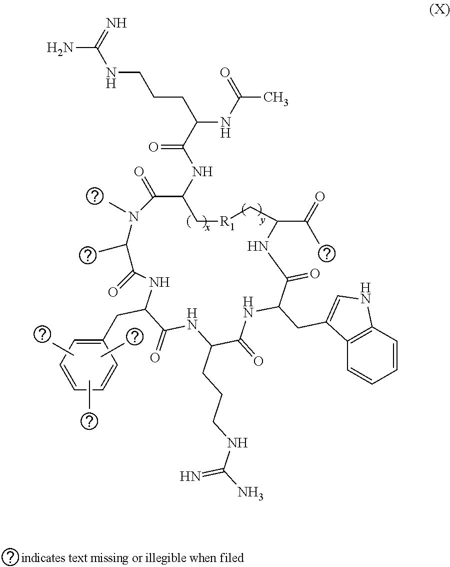

or a pharmaceutically acceptable salt thereof. In structural formula (X), the chemical substituents are defined as follows:
R¹ is-NH—C(O)— or —C(O)—NH—;
R² is-H, —CH₂—, or, R², together with R³, forms a pyrrolidine ring optionally substituted with —OH;
R³ is —(CH₂)₂— if R² is —CH₂—, and otherwise R³ is selected from

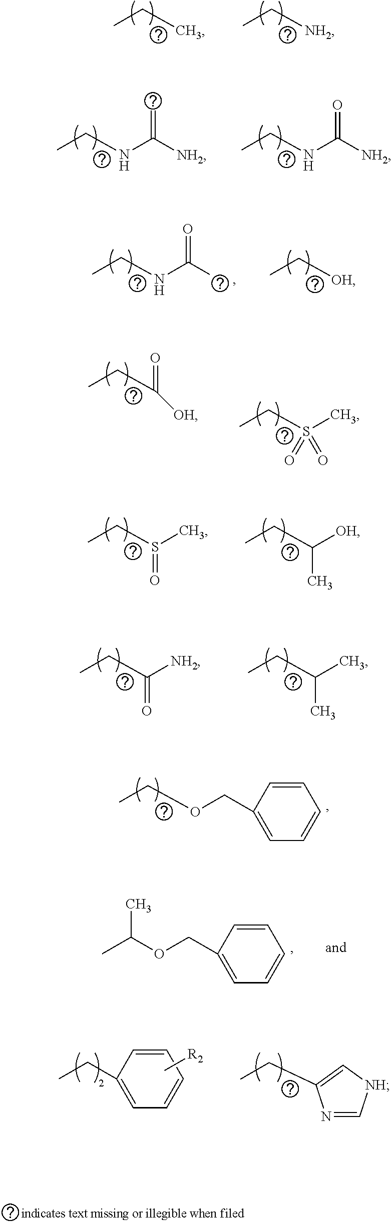

R₄ₐ, R_{4b}, and R_{4c} are each independently selected from hydrogen, halo, (C₁-C₁₀)alkyl-halo, (C₁-C₁₀)alkyl-dihalo, (C₁-C₁₀)alkyl-trihalo, (C₁-C₁₀)alkyl, (C₁-C₁₀)alkoxy, (C₁-C₁₀)alkylthio, aryl, aryloxy, nitro, nitrile, sulfoniamide, amino, hydroxyl, carboxy, and akoxy-carbonyl. In one example embodiment, R⁴a, R_{4b}, and R_{4c} is not hydrogen.
R₅ is —OH or —N(R₆ₐ)(R_{6b});
R₆ₐ and R_{6b} are each independently H or C₁ to C₄ linear, branched or cyclic alkyl chain;
R⁷ is —H or —C(O)—NH₂;
w is in each instance independently 0 to 5;
x is 1 to 5;
y is 1 to 5;
z is in each instance independently 1 to 5.

An example of a compound of structural formula (X) is a cyclic peptide defined by structural formula (XI):

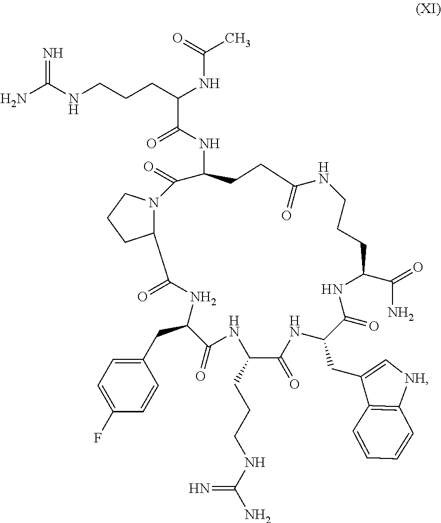 [SMILES: CC(=O)NC(CCCNC(=N)N)C(=O)N[C@H]1CCC(=O)NCCC[C@@H](C(N)=O)NC(=O)[C@H](Cc2c[nH]c3ccccc23)NC(=O)[C@H](CCCNC(=N)N)NC(=O)[C@@H](Cc2ccc(F)cc2)NC(=O)C2CCCN2C1=O]

or a pharmaceutically acceptable salt thereof.

### Patient Selection

The present disclosure features a method of treating chronic kidney disease in a subject comprising administration of an MC4R agonist (e.g., as described herein). In some embodiments, the subject has chronic kidney disease prior to administration of an MC4R agonist described herein (e.g., a compound of any one of Formulas (I), (II), (III), (IV), (V), (VI), (VII), (VIII), (X), or (XI)). In some embodiments, the subject has chronic kidney disease at the time the MC4R agonist is prescribed (e.g., a compound of any one of Formulas (I), (II), (III), (IV), (V), (VI), (VII), (VIII), (X), or (XI)). In some embodiments, the subject has chronic kidney disease at the time of the first administration of the MC4 agonist (e.g., a compound of any one of Formulas (I), (II), (III), (IV), (V), (VI), (VII), (VIII), (X), or (XI)).

In some embodiments, a subject having CKD has at least 1, 2, 3, 4, 5, 6, 7, 8, 9, or 10 of the following symptoms: high blood pressure, accumulation of urea (e.g., azotemia or uremia), hyperkalemia, decrease in erythropoietin synthesis, edema, iron deficiency anemia, metabolic acidosis, chronic kidney disease-mineral bone disorder, hypocalcemia, calciphylaxis, hyperphosphatemia, atherosclerosis, cardiovascular disease, and sexual dysfunction.

In some embodiments, a subject having CKD has a high level of a uremic toxin in the blood. Exemplary uremic toxins include urea 2-heptenal, 2-hexenal, 2-nonenal, 2-octenal, 4-decanal, anthranilic acid, argininic acid, cysteine, and dimethylamine. In some embodiments, a subject having CKD has at least 1.5%, 2%, 3%, 5%, 7.5%, 10%, 12.5%, 15%, 20%, 25%, 30%, 40%, 50%, or more of a uremic toxin level in the blood compared with a reference value.

In some embodiments, the subject having CKD has a glomerular filtration rate (GFR) of less than 75 mL/min, 70 mL/min, 65 mL/min, 60 mL/min, 55 mL/mon, 50 mL/mmin, 45 mL/min, 40 mL/min, 35 mL/min, 30 mL/min, 25 mL/min, 20 mL/min, or less. In some embodiments, the subject having CKD has a GFR about 1.5%, 2%, 3%, 5%, 7.5%, 10%, 12.5%, 15%, 20%, 25%, 30%, 40%, or 50% less than a reference value.

In some embodiments, the subject having CKD has polyuria with low urine osmolality. Urine osmolality refers to the number of dissolved particles per unit of water in the urine. A healthy subject may have a urine osmolality of between 500-800 mOsm/kg water, while a subject having CKD or other related condition may have a level lower than 500 mOsm/kg water. In some embodiments, the subject has a reduction in urine osmolality of about 1%, 2.5%, 5%, 10%, 15%, 20%, 25%, 30%, 35%, 40%, 45%, 50%, 55%, 60%, 65%, 70%, 75% or more, compared with a reference value.

In some embodiments, the subject is obese. In some embodiments, the subject is obese prior to administration of an MC4R agonist described herein (e.g., a compound of any one of Formulas (I), (II), (III), (IV), (V), (VI), (VII), (VIII), (X), or (XI)). In some embodiments, the subject is obese at the time the MC4R agonist is prescribed (e.g., a compound of any one of Formulas (I), (II), (III), (IV), (V), (VI), (VII), (VIII), (X), or (XI)). In some embodiments, the subject is obese at the time of the first administration of the MC4 agonist (e.g., a compound of any one of Formulas (I), (II), (III), (IV), (V), (VI), (VII), (VIII), (X), or (XI)).

In some embodiments, the subject has Bardet-Beidl syndrome (BBS). In some embodiments, the subject has BBS prior to administration of an MC4R agonist described herein (e.g., a compound of any one of Formulas (I), (II), (III), (IV), (V), (VI), (VII), (VIII), (X), or (XI)). In some embodiments, the subject has BBS at the time the MC4R agonist is prescribed (e.g., a compound of any one of Formulas (I), (II), (III), (IV), (V), (VI), (VII), (VIII), (X), or (XI)). In some embodiments, the subject has BBS at the time of the first administration of the MC4 agonist (e.g., a compound of any one of Formulas (I), (II), (III), (IV), (V), (VI), (VII), (VIII), (X), or (XI)).

In some embodiments, the subject has Alstrom syndrome (ALMS). In some embodiments, the subject has ALMS prior to administration of an MC4R agonist described herein (e.g., a compound of any one of Formulas (I), (II), (III), (IV), (V), (VI), (VII), (VIII), (X), or (XI)). In some embodiments, the subject has ALMS at the time the MC4R agonist is prescribed (e.g., a compound of any one of Formulas (I), (II), (III), (IV), (V), (VI), (VII), (VIII), (X), or (XI)). In some embodiments, the subject has ALMS at the time of the first administration of the MC4 agonist (e.g., a compound of any one of Formulas (I), (II), (III), (IV), (V), (VI), (VII), (VIII), (X), or (XI)).

In embodiments, the subject (e.g., adult subject) has a body mass index (BMI) greater than 25 kg/m² or 30 kg/m² (e.g., ≥25, 26, 27, 28, 29, 30, 31, 32, 33, 34, 35, 36, 37, 38, 39, 40, 41, 42, 43, 44, 45, 46, 47, 48, 49, 50 kg/m² or greater) prior to administration of the agonist, e.g., at the time the agonist is prescribed, or at the time of the first administration.

In embodiments, the subject (e.g., adult subject) has a body mass index (BMI) higher than 85-95 percentile prior to administration of the agonist, e.g., at the time the agonist is prescribed, or at the time of the first administration.

In embodiments, the subject has a body weight of at least about 5 kg, e.g., at least about 5 kg, 10 kg, 20 kg, 30, 40, 50, 60, 70, 80, 90, 100, 110, 120, 130, 140,145, 150, 155, 160, 165, 170, 175, 180, 185, 190, 200, 205, 210, 215, 220 kg or greater, e.g., prior to administration of the agonist, e.g., at the time the agonist is prescribed, or at the time of the first administration. In embodiments, the subject has a body weight of a least 20 kg, at least 60 kg, or at least 100 kg, e.g., prior to administration of the agonist, e.g., at the time the agonist is prescribed, or at the time of the first administration.

In some embodiments, the subject has uncontrolled polyuria with low urine osmolality. In some embodiments, the subject has a reduction in urine osmolality of about 1%, 2.5%, 5%, 10%, 15%, 20%, 25%, 30%, 35%, 40%, 45%, 50%, 55%, 60%, 65%, 70%, 75% or more compared with a reference value.

In some embodiments, the subject has failed one or more previous therapies, e.g., exercise, diet, or behavioral therapies, prior to administration of anMC4R agonist (e.g., a compound of any one of Formulas (I), (II), (III), (IV), (V), (VI), (VII), (VIII), (X), or (XI)), e.g., prior to administration of the agonist, e.g., at the time the agonist is prescribed, or at the time of the first administration.

In some embodiments, the subject has an increased level of a biomarker relative to a reference level, e.g., prior to administration of an MC4R agonist. In some embodiments, the subject has a decreased level of a biomarker relative to a reference level, e.g., after administration of an MC4R agonist. Exemplary biomarkers include leptin, creatine, adiponectin, albumin, and an inflammatory cytokine (e.g., a pro-inflammatory cytokine). In some embodiments, the biomarker is a pro-inflammatory cytokine, such as interleukin-1 (IL-1), IL-6, IL-12, IL-18, IL-23, tumor necrosis factor (TNF), interferon gamma (IFN-gamma), a granulocyte-macrophage colony stimulating factor, or MCP-1. In some embodiments, the pro-inflammatory cytokine is selected from IL-6, MCP-1, and IL-23. In some embodiments, the biomarker is a marker of renal function, such as creatinine, urea, uric acid, or an electrolyte. In some embodiments, the biomarker is GFR. In some embodiments, the biomarker is urine osmolality.

In some embodiments, the biomarker is a structural abnormality. In some embodiments, the subject has a structural abnormality in the kidney. The structural abnormality in the kidney may comprise a fetal lobulation, a parenchymal cyst, a calyceal cyst, calyceal clubbing, or renal agenesia.

The method described herein may further comprise acquiring the level of a biomarker and/or comparing the acquired level to a reference value. In some embodiments, responsive to the comparison, an MC4R agonist (e.g., compound of any one of Formulas (I), (II), (III), (IV), (V), (VI), (VII), (VIII), (X), or (XI)) is administered to the subject. In some embodiments, a dosage or treatment comprising an MC4R agonist (e.g., a compound of any one of Formulas (I), (II), (III), (IV), (V), (VI), (VII), (VIII), (X), or (XI)) is administered responsive to the level of a biomarker.

In some embodiments, a reference level or a reference value used in a method described herein may include a level of a biomarker in a subject or a level of a biomarker as described in the art (e.g., a reference standard). In some embodiments, a reference level or reference value used in any method described herein includes an outcome, e.g., outcome described herein, of a chronic kidney disease therapy. In some embodiments, a reference level or reference value is a level of a biomarker in the subject prior to initiation of a therapy. In some embodiments, a reference level is a measure of presence of, progression of, or severity of chronic kidney disease or a co-morbidity thereof, e.g., or the presence of or severity of symptoms of disease prior to initiation of a therapy, e.g., an MC4R agonist described herein.

In some embodiments, the amount of a dosage or treatment comprising an MC4R agonist (e.g., a compound of any one of Formulas (I), (II), (III), (IV), (V), (VI), (VII), (VIII), (X), or (XI)) is sufficient to decrease the level of a biomarker relative to a reference value.

In some aspects, provided herein is also a method of evaluating a subject, e.g., for likely responsiveness to a MC4R agonist, e.g., a MC4R agonist described herein, e.g., setmelanotide.

In embodiments, the subject is an adult, e.g., 18 years of age or older, e.g., 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or older.

In embodiments, the subject is a pediatric subject, e.g., less 18 years of age or younger (e.g., 18, 17, 16, 15, 14, 13, 12, 11, 10, 9, 8, 7, 6, 5, 4, 3, 2, or 1 year of age or younger.

### Pharmaceutical Compositions, Kits, and Administration

The present disclosure further comprises pharmaceutical compositions comprising the MC4R agonists (e.g., compounds of any one of Formulas (I), (II), (III), (IV), (V), (VI), (VII), (VIII), (X), or (XI) described herein) for the treatment of chronic kidney disease, as well as kits thereof.

In some embodiments, a pharmaceutical composition comprises an MC4R agonist (e.g., a compound of any one of Formulas (I), (II), (III), (IV), (V), (VI), (VII), (VIII), (X), or (XI) described herein or a pharmaceutically acceptable salt thereof), as well as a pharmaceutically acceptable excipient. In some embodiments, the MC4R agonists (e.g., compounds of any one of Formulas (I), (II), (III), (IV), (V), (VI), (VII), (VIII), (X), or (XI) described herein) are provided in an effective amount in the pharmaceutical composition. In some embodiments, the effective amount is a therapeutically effective amount. In some embodiments, the effective amount is a prophylactically effective amount.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the disclosure are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutical compositions described herein can be prepared by any method known in the art of pharmacology. In general, such preparatory methods include the steps of bringing the MC4R agonist (i.e., “the active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

Administration of an MC4R agonist (e.g., a compound of any one of Formulas (I), (II), (III), (IV), (V), (VI), (VII), (VIII), (X), or (XI) described herein or a pharmaceutically acceptable salt thereof) or a composition thereof can be continuous, hourly, four times daily, three time daily, twice daily, once daily, once every other day, twice weekly, once weekly, once every two weeks, once a month, or once every two months, or longer or some other intermittent dosing regimen.

Examples of administration of a compound or composition comprising an MC4R agonist (e.g., a compound of any one of Formulas (I), (II), (III), (IV), (V), (VI), (VII), (VIII), (X), or (XI) described herein or a pharmaceutically acceptable salt thereof) include peripheral administration. Examples of peripheral administration include oral, subcutaneous, intraperitoneal, intramuscular, intravenous, rectal, transdermal or intranasal forms of administration.

As used herein, peripheral administration can include all forms of administration of a compound or a composition comprising a compound of the instant invention which excludes intracranial administration. Examples of peripheral administration include, but are not limited to, oral, parenteral (e.g., intramuscular, intraperitoneal, intravenous or subcutaneous injection, extended release, slow release implant, depot and the like), nasal, vaginal, rectal, sublingual or topical routes of administration, including transdermal patch applications and the like.

In embodiments, pharmaceutical compositions, e.g., comprising an MC4R agonist described herein, can be administered with medical devices. For example, compositions comprising the agonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Other such implants, delivery systems, and modules can also be used.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The MC4R agonist (e.g., a compound of any one of Formulas (I), (II), (III), (IV), (V), (VI), (VII), (VIII), (X), or (XI) described herein or a pharmaceutically acceptable salt thereof) and related compositions described herein may be formulated in dosage unit form, e.g., single unit dosage form, for ease of administration and uniformity of dosage. It will be understood, however, that the total dosage and usage regimens of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

In some embodiments, provided herein is a unit dosage of an MC4R agonist (e.g., a compound of any one of Formulas (I), (II), (III), (IV), (V), (VI), (VII), (VIII), (X), or (XI) described herein or a pharmaceutically acceptable salt thereof). In an embodiment, the unit dosage of an MC4R agonist comprises a compound of Formula (I), e.g., setmelanotide (i.e., SEQ ID NO: 140), or a pharmaceutically acceptable salt thereof. In embodiments, the unit dosage contains 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of the agonist. The exact amount of a treatment required to achieve an effective amount will vary from subject to subject, depending, for example, on species, age, and general condition of a subject, severity of the side effects or disorder, identity of the particular MC4R agonist(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In embodiments, the MC4R agonist (e.g., a compound of any one of Formulas (I), (II), (III), (IV), (V), (VI), (VII), (VIII), (X), or (XI) described herein or a pharmaceutically acceptable salt thereof) is administered at a unit dosage, e.g., comprising 0.1-10 mg, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, or 10 mg of the agonist, e.g., subcutaneously.

In embodiments, the MC4R agonist (e.g., a compound of any one of Formulas (I), (II), (III), (IV), (V), (VI), (VII), (VIII), (X), or (XI) described herein or a pharmaceutically acceptable salt thereof) is administered in a bolus at a dose of between 0.1-10 mg, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, or 10 mg of the agonist, e.g., subcutaneously.

It will be appreciated that the MC4R agonist (e.g., a compound of any one of Formulas (I), (II), (III), (IV), (V), (VI), (VII), (VIII), (X), or (XI) described herein or a pharmaceutically acceptable salt thereof) and related compositions, as described herein, can be administered in combination with one or more additional pharmaceutical agents. The MC4R agonist (e.g., a compound of any one of Formulas (I), (II), (III), (IV), (V), (VI), (VII), (VIII), (X), or (XI) described herein or a pharmaceutically acceptable salt thereof) and related compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

Also encompassed by the disclosure are kits (e.g., pharmaceutical packs). The inventive kits may be useful for preventing and/or treating any of the diseases, disorders or conditions described herein. The kits provided may comprise an inventive pharmaceutical composition or MC4R agonist as described herein and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or MC4R agonist described herein. In some embodiments, the inventive pharmaceutical composition or MC4R agonist described herein provided in the container and the second container are combined to form one unit dosage form.

### Exemplary Enumerated Embodiments

1. A method of treating chronic kidney disease in a subject in need thereof, comprising administering a compound of any one of Formulas (I), (II), (III), (IV), (V), (VI), (VII), (VIII), (X), or (XI), e.g., as described herein, or a pharmaceutically acceptable salt thereof.

2. The method of embodiment 1, wherein the compound is a compound of Formula (I):

(R²R³)-A¹-c(A²-A³-A⁴-A⁵-A⁶-A⁷-A⁸-A9)-A¹⁰-R¹ (I),

wherein:
A¹ is Acc, HN—(CH₂) m-C(O), a L-amino acid, a D-amino acid, or deleted;
A² is Cys, D-Cys, hCys, D-hCys, Pen, D-Pen, Asp, or Glu;
A³ is Gly, Ala, β-Ala, Gaba, Aib, a D-amino acid, or deleted;
A⁴ is His, 2-Pal, 3-Pal, 4-Pal, Taz, 2-Thi, 3-Thi, or (X¹, X², X³, X⁴, X⁵) Phe;
A⁵ is D-Phe, D-1-Nal, D-2-Nal, D-Trp, D-Bal, D-(X¹, X², X³, X⁴, X⁵) Phe, L-Phe or D-(Et) Tyr;
A⁶ is Arg, hArg, Dab, Dap, Lys, Orn, or HN—CH((CH₂)ₙ—N(R⁴R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂) s-C(O), or deleted;
A⁹ is Cys, D-Cys, hCys, D-hCys, Pen, D-Pen, Dab, Dap, Orn, or Lys;
A¹⁰ is Acc, HN—(CH₂)ₜ—C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN;
or a compound of Formula (II):

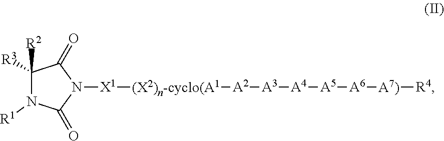

or a pharmaceutically acceptable salt thereof, wherein
X¹ is

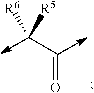

X² is

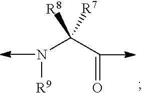

A¹ is Asp, Cys, D-Cys, Dab, Dap, Glu, Lys, Orn, Pen or D-Pen;
A² is an L- or D-amino acid;
A³ is His, 2-Pal, 3-Pal, 4-Pal, (X¹, X², X³, X⁴, X⁵) Phe, Taz, 2-Thi or 3-Thi;
A⁴ is D-Bal, D-1-Nal, D-2-Nal, D-Phe or D-(X¹, X², X³, X⁴, X⁵) Phe;
A⁵ is Arg, hArg, Dab, Dap, Lys or Orn;
A⁶ is Bal, 1-Nal, 2-Nal, (X¹, X², X³, X⁴, X⁵) Phe or Trp;
A⁷ is Asp, Cys, D-Cys, Dab, Dap, Glu, Lys, Orn, Pen or D-Pen;
R¹ is H, (C₁-C₁₀)alkyl or substituted (C₁-C₁₀)alkyl;
R² and R³ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together form a cyclic moiety;
R⁴ is OH, NH₂, CO₂H or C(O) NH₂;
R⁵ and R⁶ each is, independently, H, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R⁵ and R⁶ may be fused together form a cyclic moiety;
R⁷ and R⁸ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₆)alkyl; or R⁷ and R⁸ may be fused together form a cyclic moiety;
R⁹ is H, (C₁-C₁₀)alkyl or substituted (C₁-C₁₀)alkyl; and
n is, independently for each occurrence thereof, 0, 1, 2, 3, 4, 5, 6 or 7;
to thereby treat chronic kidney disease.

3. The method of any one of embodiment 1-2, wherein the subject has renal dysfunction.

4. The method of any one of embodiments 1-3, wherein the subject has cognitive impairment.

5. The method of any one of embodiments 1-4, wherein the subject has retinal degeneration.

6. The method of any one of embodiments 1-5, wherein the subject does not have Bardet-Biedl syndrome (BBS).

7. The method of any one of embodiments 1-6, wherein the subject has not been diagnosed with Bardet-Biedl syndrome (BBS).

8. The method of any one of embodiments 1-5, wherein the subject has Bardet-Biedl syndrome (BBS).

9. The method of any one of embodiments 1-5 and 8, wherein the subject has been diagnosed with Bardet-Biedl syndrome (BBS).

10. The method of any one of embodiments 1-8, wherein the subject has Alstrom syndrome.

11. The method of any one of embodiments 1-10, wherein the subject has been diagnosed with Alstrom syndrome.

12. The method of any one of embodiments 1-9, wherein the subject has not been diagnosed with Alstrom syndrome.

13. The method of any one of embodiments 1-12, wherein the subject is obese (e.g., severely obese).

14. The method of any one of embodiments 1-13, wherein the subject has early-onset severe obesity.

15. The method of any one of embodiments 1-14, wherein the subject is hyperphagic.

16. The method of any one of embodiments 1-15, wherein the subject has hyperleptinemia.

17. The method of any one of embodiments 1-16, wherein the subject has obesity, and/or hyperphagia, and/or hyperleptinemia, and/or Bardet-Biedl Syndrome, and/or Alstroms Syndrome and is at risk for a diagnosis of chronic kidney disease.

18. The method of any one of embodiments 1-17, wherein the subject has a body mass index (BMI) greater than 25 kg/m² (e.g., ≥25, 30, 31, 32, 33, 34, 35, 36, 37, 38, 39, 40, 41, 42, 43, 44, 45, 46, 47, 48, 49, 50 kg/m² or greater) prior to administration of the compound of any one of Formulas (I), (II), (III), (IV), (V), (VI), (VII), (VIII), (X), or (XI).

19. The method of any one of embodiments 1-17, wherein the subject has a body mass index (BMI) greater than 35 kg/m² (e.g., ≥36, 37, 38, 39, 40, 41, 42, 43, 44, 45, 46, 47, 48, 49, 50 kg/m² or greater) prior to administration of the compound of any one of Formulas (I), (II), (III), (IV), (V), (VI), (VII), (VIII), (X), or (XI).

20. The method of any one of embodiments 1-17, wherein the subject has a body mass index (BMI) greater than 45 kg/m² (e.g., ≥41, 42, 43, 44, 45, 46, 47, 48, 49, 50, 51, 52, 53, 54, 55 kg/m² or greater) prior to administration of the compound of any one of Formulas (I), (II), (III), (IV), (V), (VI), (VII), (VIII), (X), or (XI).

21. The method of any one of embodiments 1-20, wherein the subject has failed one or more previous therapies, e.g., exercise, diet, or behavioral therapies, prior to administration of the compound of any one of Formulas (I), (II), (III), (IV), (V), (VI), (VII), (VIII), (X), or (XI), e.g., at the time the compound of any one of Formulas (I), (II), (III), (IV), (V), (VI), (VII), (VIII), (X), or (XI) is prescribed, or at the time of the first administration of the compound of any one of Formulas (I), (II), (III), (IV), (V), (VI), (VII), (VIII), (X), or (XI).

22. The method of any one of embodiments 1-21, wherein prior to administration of a compound of any one of Formulas (I), (II), (III), (IV), (V), (VI), (VII), (VIII), (X), or (XI), the subject has an increased level of a biomarker relative to a reference level.

23. The method of any one of embodiments 1-22, wherein after administration of a compound of any one of Formulas (I), (II), (III), (IV), (V), (VI), (VII), (VIII), (X), or (XI), the subject has a decreased level of a biomarker relative to a reference level.

24. The method of any one of embodiments 1-23, further comprising acquiring the level of a biomarker.

25. The method of embodiment 24, further comprising comparing the acquired level to a reference value.

26. The method of embodiment 25, wherein responsive to the comparison, administering the compound of any one of Formulas (I), (II), (III), (IV), (V), (VI), (VII), (VIII), (X), or (XI).

27. The method of any one of embodiments 24-26, wherein a dosage or treatment comprising a compound of any one of Formulas (I), (II), (III), (IV), (V), (VI), (VII), (VIII), (X), or (XI) is administered responsive to the level of a biomarker.

28. The method of any one of embodiments 24-27, wherein the amount of a dosage or treatment comprising a compound of any one of Formulas (I), (II), (III), (IV), (V), (VI), (VII), (VIII), (X), or (XI) is sufficient to decrease the level of a biomarker relative to a reference value.

29. The method of any one of embodiments 1-28, wherein the biomarker is leptin.

30. The method of any one of embodiments 1-28, wherein the biomarker is creatine.

31. The method of any one of embodiments 1-28, wherein the biomarker is adiponectin.

32. The method of any one of embodiments 1-28, wherein the biomarker is an inflammatory cytokine.

33. The method of embodiment 32, wherein the inflammatory cytokine is a pro-inflammatory cytokine.

34. The method of embodiment 33, wherein the pro-inflammatory cytokine comprises IL-6, MCP-1, or IL-23.

35. The method of any one of embodiments 1-28, wherein the biomarker is a structural abnormality.

36. The method of embodiment 35, wherein the subject has a structural abnormality in the kidney.

37. The method of any one of embodiments 35-36, wherein the structural abnormality comprises a fetal lobulation, a parenchymal cyst, a calyceal cyst, calyceal clubbing, or renal agenesia.

38. The method of any one of embodiments 1-37, wherein the subject is a mammal, e.g., a human.

39. The method of any one of embodiments 1-38, wherein the compound is a compound of Formula (I) or a pharmaceutically acceptable salt thereof.

40. The method of any one of embodiments 1-39, wherein the compound of Formula (I) is Ac-Arg-c (Cys-D-Ala-His-D-Phe-Arg-Trp-Cys)-NH₂ (SEQ ID NO: 140).

41. The method of any one of embodiments 1-38, wherein the compound is a compound of Formula (II) or a pharmaceutically acceptable salt thereof.

42. The method of any one of embodiments 1-38 and 41, wherein the compound of Formula (II) is Hydantoin (C(O)-(Arg-Gly))-cyclo(Cys-Glu-His-D-Phe-Arg-Trp-Cys)-NH₂ (SEQ ID NO: 13).

43. The method of any one of embodiments 1-42, wherein the compound is a compound of Formula (I) or Formula (II).

44. The method of any one of embodiments 1-43, wherein the compound of any one of Formulas (I), (II), (III), (IV), (V), (VI), (VII), (VIII), (X), or (XI) is formulated as a pharmaceutical composition.

45. The method of any one of embodiments 1-44, wherein the compound of Formula (I) or Formula (II) is formulated as a pharmaceutical composition.

46. A method of evaluating a subject for treatment of chronic kidney disease comprising: acquiring the level of a biomarker, e.g., leptin, creatine, adiponectin, an inflammatory cytokine (e.g., a pro-inflammatory cytokine, e.g., IL-6, MCP-1, or IL-23), or a structural abnormality.

47. The method of embodiment 46, wherein the treatment comprises administration of a compound of any one of Formulas (I), (II), (III), (IV), (V), (VI), (VII), (VIII), (X), or (XI), or a pharmaceutically acceptable salt thereof.

48. The method of any one of embodiment 46-47, wherein the treatment comprises the administration of a compound of Formula (I) or Formula (II), or a pharmaceutically acceptable salt thereof.

49. The method of any one of embodiments 46-48, further comprising comparing the acquired level of a biomarker with a reference value.

50. The method of embodiment 49, responsive to the comparison administering the compound of any one of Formulas (I), (II), (III), (IV), (V), (VI), (VII), (VIII), (X), or (XI), or a pharmaceutically acceptable salt thereof.

51. The method of any of embodiments 46-50, wherein a dosage or treatment of a compound of any one of Formulas (I), (II), (III), (IV), (V), (VI), (VII), (VIII), (X), or (XI) is administered responsive to the level of the biomarker.

52. The method of any one of embodiments 46-51, wherein the biomarker is leptin.

53. The method of any one of embodiments 46-51, wherein the biomarker is creatine.

54. The method of any one of embodiments 46-51, wherein the biomarker is adiponectin.

55. The method of any one of embodiments 46-51, wherein the biomarker is an inflammatory cytokine.

56. The method of embodiment 55, wherein the inflammatory cytokine is a pro-inflammatory cytokine.

57. The method of embodiment 56, wherein the pro-inflammatory cytokine comprises IL-6, MCP-1, or IL-23.

58. The method of any one of embodiments 46-51, wherein the biomarker is a structural abnormality.

59. The method of embodiment 58, wherein the subject has a structural abnormality in the kidney.

60. The method of any one of embodiments 58-59, wherein the structural abnormality comprises a fetal lobulation, a parenchymal cyst, a calyceal cyst, calyceal clubbing, or renal agenesia.

61. The method of any one of embodiments 46-60, wherein the compound is a compound of Formula (I) or a pharmaceutically acceptable salt thereof.

62. The method of any one of embodiments 46-61, wherein the compound of Formula (I) is Ac-Arg-c (Cys-D-Ala-His-D-Phe-Arg-Trp-Cys)-NH₂ (SEQ ID NO: 140).

63. The method of any one of embodiments 46-60, wherein the compound is a compound of Formula (II) or a pharmaceutically acceptable salt thereof.

64. The method of any one of embodiments 46-60 and 63, wherein the compound of Formula (II) is Hydantoin (C(O)-(Arg-Gly))-cyclo(Cys-Glu-His-D-Phe-Arg-Trp-Cys)-NH₂ (SEQ ID NO: 13).

65. The method of any one of embodiments 46-64, wherein the compound of any one of Formulas (I), (II), (III), (IV), (V), (VI), (VII), (VIII), (X), or (XI) is formulated as a pharmaceutical composition.

66. The method of embodiment 65, wherein the compound of Formula (I) or Formula (II) is formulated as a pharmaceutical composition.

### Examples

In order that the disclosure described herein may be more fully understood, the following examples are set forth. The examples described in this application are offered to illustrate the methods of treating chronic kidney disease in a subject as provided herein and are not to be construed in any way as limiting their scope.

### Example 1: Setmelanotide Lowers Renal IL-23 Levels and Plasma Leptin Levels

In order to assess the effect of setmelanotide on plasma leptin levels disproportionate from body weight loss, BBS10^{-/-} and wild type mice were fed a high fat/high glucose diet from weaning and body weight assessed on a weekly basis. At four months of age, mice were transferred to metabolic cages and acclimated for 3 days. An escalating setmelanotide dosing study over 10-days was conducted in accordance with regimen shown below in Table 1:

TABLE 1
Parameters for setmelanotide dosing study in BBS 10−/− and wild type mice

| | No. of | | | | | | | | |
|---|---|---|---|---|---|---|---|---|---|
| Group | Animals | | | Day 2 | Day 4 | Day 6 | Day 8 | | Volume |
| No. | (Males) | Strain | Day 1 | (mg/kg) | (mg/kg) | (mg/kg) | (mg/kg) | Day 10 | (ml/kg) |
| 1 | 8 | WT | Vehicle | 0.05 | 0.125 | 0.3125 | 1.25 | Vehicle | 5 |
| 2 | 8 | BBS10^{-/-} | Vehicle | 0.05 | 0.125 | 0.3125 | 1.25 | Vehicle | 5 |

At the completion of the study, mice were sacrificed and blood and kidney collected for analysis of circulating leptin concentration and IL-23 mRNA expression, respectively. Plasma leptin concentrations were determined using a leptin ELISA kit (Catalog: #: EZML-82K, Millipore, Billerica, Massachusetts, USA). For analysis of renal IL-23 expression messenger RNA was extracted from tissue samples using TRIzol® reagent (#15596-018, Life Technologies, USA). The reverse transcription reaction was performed after a DNAse treatment, using the iScript® cDNA synthesis kit (#170-889, BioRad, USA) and the Mastercycler® thermocycler (#: 22331, Eppendorf, USA). The real-time quantitative PCR reaction was performed in a C₁₀₀₀™ thermocycler (CFX96, Real-Time System, BioRad, USA) using SYBR® Green. Primers sequences for IL-23 were 5′-TGCTGGATTGCAGAGCAGTAA and 3′-GCATGCAGAGATTCCGAGAGA (Goto et al 2015). Results were analyzed with the CFX Manager™ software (BioRad, USA). Gene expression quantification was assessed by reporting measured levels relative to Gapdh expression in each separate sample.

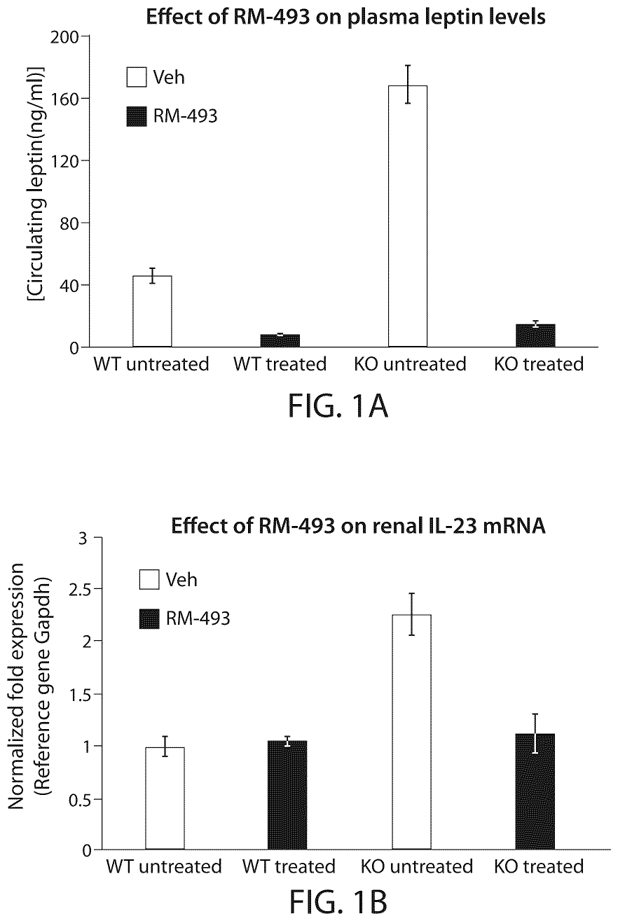

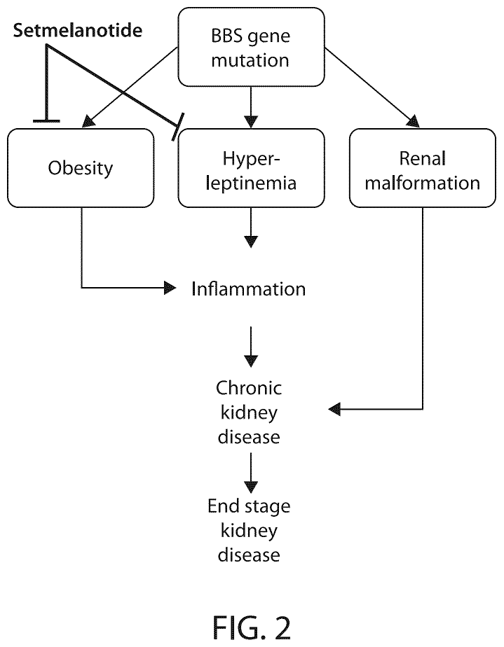

The results demonstrate that setmelanotide can decrease plasma leptin levels in a manner disproportionate from body weight loss in BBS10^{-/-} mice on high-fat/high-glucose diet (FIG. 1). Furthermore, this was associated with a normalization of renal IL-23 mRNA expression. The mechanism underlying melanocortinergic (body-weight independent) leptin regulation is presently unknown. However, these data indicate setmelanotide may improve CKD progression in BBS through body weight dependent and independent mechanisms of leptin lowering (FIG. 2).

### Example 2: Setmelanotide as a Treatment for Leptin-Mediated Renal Dysfunction

The following study is designed to assess the potential for setmelanotide as a treatment for chronic kidney disease, specifically leptin-mediated renal dysfunction. It was hypothesized that setmelanotide administration would decrease body weight and circulating leptin levels in HF/HG diet fed BBS10^{-/-} mice (in a manner disproportionate to adipose tissue loss) leading to reduced expression of renal inflammatory mediators and improved renal function. Positive outcomes for these endpoints would highlight a role for setmelanotide in the treatment, and possible prevention, of progressive CKD, for example in BBS and other co-morbidities (e.g., other ciliopathies).

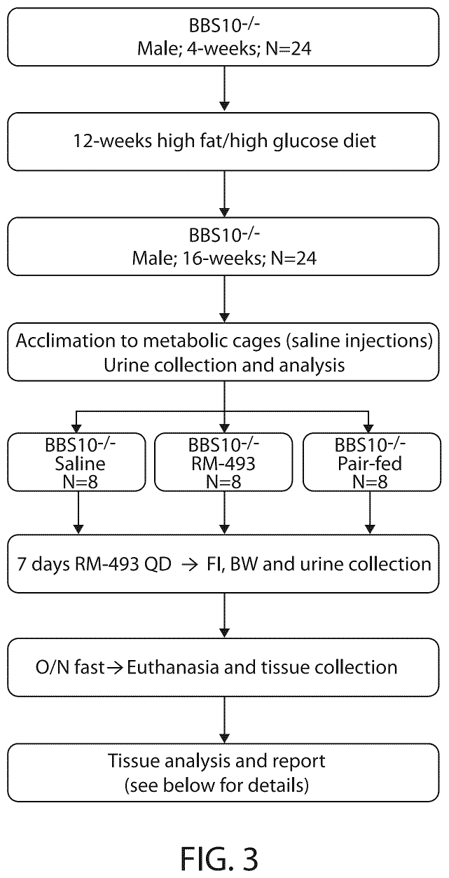

At weaning 24 male BBS10^{-/-} mice will be transferred to a high fat and high glucose diet. After 12 weeks of HF/HG feeding mice will be singly housed for 3 days and urine from now obese BBS10^{-/-} mice will be collected for analysis (see study design in FIG. 3). Mice will subsequently be separated into three groups (ensuring average urine creatinine level is comparable across groups: Group 1-vehicle; Group 2-setmelanotide 1.25 mg/kg; Group 3-pair-fed to Group 2). Mice will be dosed daily for 7 days (just before onset of the dark cycle) and 24 h body weight and cumulative daily food intake recorded at the time of administration (dose administration will occur at the same time of day each for 7 days, before start of the dark cycle). After 7 days urine will be collected for analysis, see details below. Mice will then be dosed with SET [last dose] and euthanized by live decapitation the following morning. Fasting blood glucose will be recorded. Tissues will be harvested and stored: trunk blood (for plasma); kidneys, WAT, heart and liver.

Analyses will include the following:
Urine—Creatine, urea, NA, K, C₁, calcium, phosphorus, total protein, albumin, glucose and osmolarity;
Plasma—Metabolic Luminex panel (to include: leptin, adiponectin and insulin); cytokine Luminex panel (to include: IL-1b, IL-4, IL-6, IL-10, IL-12, IL-13, IL-17, IL-23, TNFa, IFNg, eotaxin); CRP Elisa; creatinine, Na, K, C₁ (for determination of clearance and reabsorption) and glucose;
Kidney—(R) H&E histology; Tunnel assay; CD68-IR (infiltration assay) (L) cytokine mRNA expression (to include IL-23);
WAT—CD68-IR; cytokine, adipokine mRNA expression;
Heart—Cytokine, adipokine mRNA expression; and
Liver—CD68-IR; cytokine mRNA expression.

### Example 3: Setmelanotide Treatment Results in Lowered Body Weight and Affects Biomarker Levels

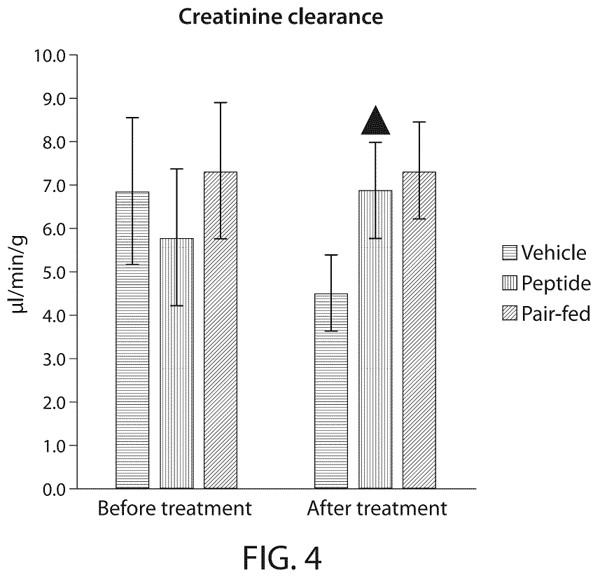

In order to assess the effect of setmelanotide on body weight and the levels of certain biomarkers, BBS10^{-/-} mice were administered setmelanotide (1.25 mg/kg) daily for one week. Setmelanotide treatment did not alter the blood plasma levels of several biomarkers, including glucose, sodium, potassium, chloride, calcium, phosphorus and creatine, compared to mice treated with vehicle or which were pair-fed. However, setmelanotide treatment did result in an increase in urine calcium levels as well as an increase in urine creatine levels (FIG. 4).

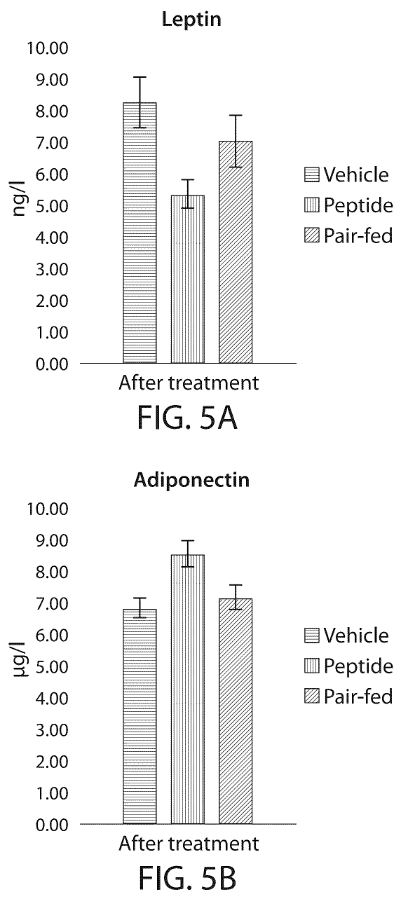

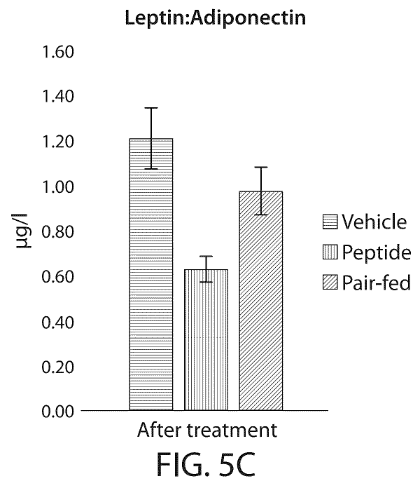

Additional studies in these mice entailed measurement of blood plasma levels of the biomarkers leptin and adiponectin after treatment with setmelanotide (1.25 mg/kg). As shown in FIGS. 5A-5C, setmelanotide treatment resulted in decreasing levels of leptin and increased levels of adiponectin. Further, the leptin: adiponectin ration was decreased in setmelanotide treated mice. Vehicle and pair-fed BBS10^{-/-} mice did not exhibit these effects, suggesting a setmelanotide-dependent and body weight-independent mechanism.

### Equivalents and Scope

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A method for treating chronic kidney disease in a subject in need thereof, comprising administering a compound of Formula (I):
(R²R³)-A¹-c(A²-A³-A⁴-A⁵-A⁶-A⁷-A⁸-A9)-A¹⁰-R¹ (I),
wherein:
A¹ is Acc, HN—(CH₂)ₘ—C(O), a L-amino acid, a D-amino acid, or deleted;
A² is Cys, D-Cys, hCys, D-hCys, Pen, D-Pen, Asp, or Glu;
A³ is Gly, Ala, β-Ala, Gaba, Aib, a D-amino acid, or deleted;
A⁴ is His, 2-Pal, 3-Pal, 4-Pal, Taz, 2-Thi, 3-Thi, or (X¹, X², X³, X⁴, X⁵) Phe;
A⁵ is D-Phe, D-1-Nal, D-2-Nal, D-Trp, D-Bal, D-(X¹, X², X³, X⁴, X⁵) Phe, L-Phe or D-(Et) Tyr;
A⁶ is Arg, hArg, Dab, Dap, Lys, Orn, or HN—CH((CH₂)ₙ—N(R⁴ R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂) s-C(O), or deleted;
A⁹ is Cys, D-Cys, hCys, D-hCys, Pen, D-Pen, Dab, Dap, Orn, or Lys;
A¹⁰ is Acc, HN—(CH₂)ₜ—C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN;
or a compound of Formula (II): 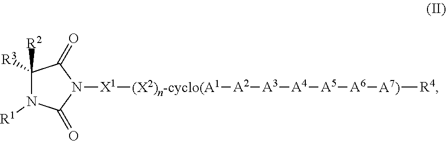
or a pharmaceutically acceptable salt thereof, wherein
X¹ is 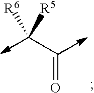
X² is 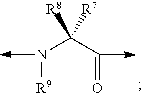
A¹ is Asp, Cys, D-Cys, Dab, Dap, Glu, Lys, Orn, Pen or D-Pen;
A² is an L- or D-amino acid;
A³ is His, 2-Pal, 3-Pal, 4-Pal, (X¹, X², X³, X⁴, X⁵) Phe, Taz, 2-Thi or 3-Thi;
A⁴ is D-Bal, D-1-Nal, D-2-Nal, D-Phe or D-(X¹, X², X³, X⁴, X⁵) Phe;
A⁵ is Arg, hArg, Dab, Dap, Lys or Orn;
A⁶ is Bal, 1-Nal, 2-Nal, (X¹, X², X³, X⁴, X⁵) Phe or Trp;
A⁷ is Asp, Cys, D-Cys, Dab, Dap, Glu, Lys, Orn, Pen or D-Pen;
R¹ is H, (C₁-C₁₀)alkyl or substituted (C₁-C₁₀)alkyl;
R² and R³ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together form a cyclic moiety;
R⁴ is OH, NH₂, CO₂H or C(O) NH₂;
R⁵ and R⁶ each is, independently, H, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R⁵ and R⁶ may be fused together form a cyclic moiety;
R⁷ and R⁸ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₆)alkyl; or R⁷ and R⁸ may be fused together form a cyclic moiety;
R⁹ is H, (C₁-C₁₀)alkyl or substituted (C₁-C₁₀)alkyl; and
n is, independently for each occurrence thereof, 0, 1, 2, 3, 4, 5, 6 or 7;
to thereby treat chronic kidney disease.

2. The method of claim 1, wherein the subject has renal dysfunction, cognitive impairment, or retinal degeneration.

3-5. (canceled)

6. The method of claim 1, wherein the subject has Bardet-Biedl syndrome (BBS).

7. The method of claim 1, wherein the subject has Alstrom syndrome.

8. The method of claim 1, wherein the subject is:
(i) obese (e.g., severely obese);
(ii) hyperphagic; and/or
(iii) has hyperleptinemia.

9-10. (canceled)

11. The method of claim 1, wherein the subject has obesity, and/or hyperphagia, and/or hyperleptinemia, and/or Bardet-Biedl Syndrome, and/or Alstrom Syndrome and is at risk for a diagnosis of chronic kidney disease.

12. The method of claim 1, wherein the subject:
(i) has a body mass index (BMI) greater than 35 kg/m² (e.g., ≥36, 37, 38, 39, 40, 41, 42, 43, 44, 45, 46, 47, 48, 49, 50 kg/m² or greater) prior to administration of the compound of Formula (I) or Formula (II); or
(ii) the subject has failed one or more previous therapies, e.g., exercise, diet, or behavioral therapies, prior to administration of the compound of Formula (I) or Formula (II), e.g., at the time the compound of Formula (I) or Formula (II) is prescribed, or at the time of the first administration of the compound of Formula (I) or Formula (II).

13. (canceled)

14. The method of claim 1, wherein prior to administration of a compound of Formula (I) or Formula (II), the subject has an increased level of a biomarker relative to a reference level.

15-18. (canceled)

19. The method of claim 14, wherein a dosage or treatment comprising a compound of Formula (I) or Formula (II) is administered responsive to the level of a biomarker.

20. (canceled)

21. The method of claim 19, wherein the biomarker is leptin, adiponectin, creatine, an inflammatory cytokine, or a structural abnormality.

22. The method of claim 21, wherein the subject has a structural abnormality in the kidney.

23. The method of claim 1, wherein the subject is a mammal, e.g., a human.

24. (canceled)

25. The method of claim 1, wherein the compound of Formula (I) is Ac-Arg-c (Cys-D-Ala-His-D-Phe-Arg-Trp-Cys)-NH₂ (SEQ ID NO: 140).

26. (canceled)

27. The method of claim 1, wherein the compound of Formula (II) is Hydantoin (C(O)-(Arg-Gly))-cyclo(Cys-Glu-His-D-Phe-Arg-Trp-Cys)-NH₂ (SEQ ID NO:13).

28. The method of claim 1, wherein the compound of Formula (I) or Formula (II) is formulated as a pharmaceutical composition.

29. A method for treating a subject having chronic kidney disease comprising:
acquiring the level of a biomarker, e.g., leptin, creatine, adiponectin, an inflammatory cytokine (e.g., a pro-inflammatory cytokine, e.g., IL-6, MCP-1, or IL-23), or a structural abnormality, and
administering to the subject, responsive to the level of the biomarker, a compound of Formula (I) or Formula (II), or a pharmaceutically acceptable salt thereof.

30-35. (canceled)

36. The method of claim 29, wherein the structural abnormality comprises a fetal lobulation, a parenchymal cyst, a calyceal cyst, calyceal clubbing, or renal agenesia.

37. (canceled)

38. The method of claim 29, wherein the compound of Formula (I) is Ac-Arg-c (Cys-D-Ala-His-D-Phe-Arg-Trp-Cys)-NH₂ (SEQ ID NO: 140).

39. (canceled)

40. The method of claim 29, wherein the compound of Formula (II) is Hydantoin (C(O)-(Arg-Gly))-cyclo(Cys-Glu-His-D-Phe-Arg-Trp-Cys)-NH₂ (SEQ ID NO:13).

41. (canceled)
